(19) United States
(12) Patent Application Publication
(10) Pub. No.: US 2012/0208746 A1
(43) Pub. Date: Aug. 16, 2012

(54) GENETIC MARKERS ASSOCIATED WITH RESPONSE TO CYCLOPHILIN-BINDING COMPOUNDS

(75) Inventors: Samuel Earl HOPKINS, Raleigh, NC (US); Yves Joseph Ribeill, Raleigh, NC (US); Bernard Scorneaux, Cary, NC (US)

(73) Assignee: Scynexis Inc., Durham, NC (US)

(21) Appl. No.: 13/349,251

(22) Filed: Jan. 12, 2012

Related U.S. Application Data
(60) Provisional application No. 61/432,159, filed on Jan. 12, 2011.
(60) Provisional application No. 61/504,086, filed on Jul. 1, 2011.
(60) Provisional application No. 61/537,486, filed on Sep. 21, 2011.
(60) Provisional application No. 61/555,753, filed on Nov. 4, 2011.

(51) Int. Cl. A61K 38/13; A61P 31/14; C07K 7/64; C12N 5/071; C12Q 1/68

(52) U.S. Cl. 514/4.3; 435/375; 435/6.11; 530/321

(57) ABSTRACT

Methods of predicting the response in a patient infected with hepatitis C virus (HCV) to a treatment regime involving the use of a cyclophilin-binding compound are described which provide for improvements in treatments, pharmaceutical compositions, dosing regimen, assays, kits, and other aspects of the art.

### CROSS-REFERENCE TO EARLIER APPLICATIONS

This application claims benefit under 35 U.S.C. §119(e) of U.S. Provisional Applications No. 61/432,159, filed Jan. 12, 2011; No. 61/504,086, filed Jul. 1, 2011; No. 61/537,486, filed Sep. 21, 2011; and No. 61/555,753, filed Nov. 4, 2011; each of which is incorporated by reference herein in its entirety and relied upon.

### FIELD

This disclosure relates to methods of treating a patient infected with a disease susceptible to treatment with a cyclophilin binding compound, for example a patient infected with hepatitis C virus (HCV), involving the use of a cyclophilin inhibitor.

### BACKGROUND

Cyclophilins are a family of enzymes that assist in the folding and transportation of other proteins synthesized within a cell. Protein folding or misfolding plays a central role in the pathophysiology of a number of serious diseases, such as viral diseases, central nervous system disorders, cancer and cardiovascular diseases. Cyclophilin inhibitors, such as cyclosporin A, have been used for decades for the prophylaxis of organ rejection in transplant patients. Cyclophilin inhibitors are also known as cyclophilin-binding compounds.

The cyclophilin-binding compound cyclosporine A and certain derivatives have been reported as having anti-HCV activity, see Watashi et al., 2003, Hepatology 38:1282-1288, Nakagawa et al., 2004, Biochem. Biophys. Res. Commun. 313:42-7, and Shimotohno and K. Watashi, 2004, American Transplant Congress, Abstract No. 648 (American Journal of Transplantation 2004, Volume 4, Issue s8, Pages 1-653). Cyclosporine derivatives having HCV activity are described in International Patent Publication Nos. WO2005/021028, WO2006/039668, WO2006/038088, WO2007/041631, WO2008/069917, WO2010/002428, WO2010/076329, WO2010/088573. Cyclophilin-binding compounds that have been evaluated for use in the treatment of HCV include alisporivir ([8-(N-methyl-D-alanine), 9-(N-ethyl-L-valine)]cyclosporin, also known as DEBIO-025), (melle-4)cyclosporin (also known as NIM-811) and 3-[(R)-2-(N,N-dimethylamino)ethylthio-Sar]-4-(gamma-hydroxymethylleucine)cyclosporin (also known as SCY-635). Further examples of cyclophilin inhibitors include sanglifehrin A and derivatives thereof, which have been described as having activity against HCV in International Patent Publication No. WO/2006/138507.

In a recent paper by Ge et al [Nature, Vol. 461 (2009), pages 399-401] it has been reported that a polymorphism on chromosome 19 (rs12979860) was strongly associated with sustained virological response (SVR) in patient groups treated with a 48-week course of pegylated interferon-α-2b (PegIFN-α-2b) or -α-2a (PegIFN-α-2a) combined with ribavirin. The polymorphism resides 3 kilobases (kb) upstream of the IL28B gene, encoding interferon-lambda-3. The authors conclude that the IL28B polymorphism strongly influences response within each of the major population groups and appears to explain much of the difference in response between different population groups (such as European-Americans compared with African-Americans). In particular, three polymorphisms have been identified that are predictive of response to interferon: CC (78% SVR), CT (37% SVR) and TT (26% SVR). International Patent Publication No. WO2010/135649 further describes the use of this genetic marker in predicting the response of HCV-infected patients to therapy with an interferon alpha. Suppiah et al [Nature Genetics, Vol. 41 (2009), pages 1100-1104 and Tanaka et al [ibid, pages 1105-1109] also describe the genome-wide association of IL28B with response to pegylated interferon-alpha and ribavirin therapy for HCV.

### SUMMARY

Certain single nucleotide polymorphisms (SNPs) on human chromosomal region 19q13.13 are strongly associated with response to treatment by cyclophilin-binding compounds of patients chronically infected with HCV, in particular patients with genotype 1 HCV. One of these SNPs is a C/T polymorphism, identified as rs 12979860 in the NCBI SNP Database. The rs 12979860 polymorphism is located 3 kb upstream of the interleukin 28B (IL-28B) gene, which encodes interferon lambda 3 (IFN-lambda 3). The presence of the C allele is associated with a better treatment response, with the C/C genotype associated with a greater virological response than the C/T genotype, and the C/T genotype associated with a greater virological response than the T/T genotype in HCV.

This and other discoveries described herein have led to advances over the state of the art. For example, a pharmaceutical composition for treating an individual having a disease susceptible to treatment with a cyclophilin-binding compound and a positive test result for at least one cyclophilin-binding compound marker can comprise a cyclophilin-binding compound. In addition, the use of a cyclophilin-binding compound in the manufacture of a medicament for treating an individual having a disease susceptible to treatment with the cyclophilin-binding compound and a positive test for at least one cyclophilin-binding compound marker is provided.

As another example, a drug product may comprise a cyclophilin-binding compound, a pharmaceutical composition and prescribing information which includes a pharmacogenetic indication for which the pharmaceutical composition is recommended. The pharmacogenetic indication may include two components: a disease susceptible to treatment with the cyclophilin-binding compound in the pharmaceutical composition and patients who have the disease and who are genetically defined by having at least one cyclophilin-binding compound marker. A method of testing an individual for the presence or absence of at least one cyclophilin-binding compound marker may comprise obtaining a nucleic acid sample from the individual and assaying the sample to determine the individual's genotype for at least one of the polymorphic sites identified in Table 1.

After testing, an individual can be treated with a cyclophilin-binding compound in accordance with the test results. For example, a genotype result can be indicative of a susceptibility to treatment using a cyclophilin-binding compound and treatment in accordance with the test result can comprise a treatment including administration of a cyclophilin-binding compound at an effective dose. The effective dose can be reduced in the case of a test result identifying a marker of susceptibility to treatment with a cyclophilin-binding compound. Alternatively, a genotype result can be indicative of a reduced susceptibility to treatment with a cyclophilin-binding compound. In the case of a genotype indicating reduced susceptibility, a treatment in accordance with the test results can comprise administration of a cyclophilin-binding compound at an effective dose, which can be a higher dose, or can comprise a treatment with a cyclophilin-binding compound in combination with another treatment, or can comprise treatment that does not include administering a cyclophilin-binding compound.

Also provided are dosing regimens wherein a cyclophilin-binding compound, for example SCY-635, or a pharmaceutically acceptable salt, solvate or hydrate thereof, is administered at specific time intervals to treat diseases, in particular viral diseases such as HIV, and in particular HCV. Also provided herein are doses and unit dosage forms of SCY-635, or a pharmaceutically acceptable salt, solvate or hydrate thereof. A method of prescribing a dosage of SCY-635 can comprise obtaining a genotype of a patient, for example a genotype of a marker for susceptibility to treatment with a cyclophilin-binding compound, and selecting an effective dosage or dosing regimen in accordance with the genotype of the patient. Methods of treating a patient in need thereof can comprise administering a dosage or dosing regimen of SCY-635 that is selected in accordance with a genotype of the patient.

Also provided are methods for treating, preventing or managing hepatitis C virus infection in a human subject infected with, or at risk for infection with, hepatitis C virus, the method comprising administering to the human subject an effective amount of SCY-635, or a pharmaceutically acceptable salt, solvate or hydrate thereof, at least about two times in the course of a 24 hour period, wherein the subject has been determined to have a genotype indicative of susceptibility to treatment with a cyclophilin-binding compound. Also provided herein are methods for administering to an infected human subject in need thereof a pharmaceutical composition comprising an effective amount of SCY-635, or a pharmaceutically acceptable salt, solvate or hydrate thereof, at least about two or at least about three times in the course of a 24 hour period, wherein the subject has been determined to have a genotype indicative of susceptibility to treatment with a cyclophilin-binding compound.

Furthermore, methods of treating a subject in need thereof, wherein the subject has a condition, disease, or other indication that is treatable with INF-α can comprise administering a cyclophilin-binding compound, for example SCY-635. In some examples, methods of treating a subject for a disease, condition, or other indication that is susceptible to treatment with IFN-α can comprise administration of SCY-635. In some examples, IFN-α is not administered.

### BRIEF DESCRIPTION OF THE DRAWINGS

FIGS. 1(A-B) illustrate (A) OAS1 expression () over 15 days in plasma samples of a patient treated with 300 mg t.i.d. of SCY-635 over a 15 day period, and (▪) log change in HCV from baseline over the same period; and, (B) a comparison of plasma SCY-635 concentration (▪) over the study period and OAS1 expression ().

FIGS. 2(A-B) illustrate (A) OAS1 expression () over 15 days in plasma samples of a patient treated with 300 mg t.i.d. of SCY-635 over a 15 day period, and (▪) log change in HCV from baseline over the same period; and, (B) a comparison of plasma SCY-635 concentration (▪) over the study period and OAS1 expression ().

FIGS. 3(A-B) illustrate (A) OAS1 expression () over 15 days in plasma samples of a patient treated with 300 mg t.i.d. of SCY-635 over a 15 day period, and (▪) log change in HCV from baseline over the same period; and, (B) a comparison of plasma SCY-635 concentration (▪) over the study period and OAS1 expression ().

FIGS. 4(A-B) illustrate (A) OAS1 expression () over 15 days in plasma samples of a patient treated with 300 mg t.i.d. of SCY-635 over a 15 day period, and (▪) log change in HCV from baseline over the same period; and, (B) a comparison of plasma SCY-635 concentration (▪) over the study period and OAS1 expression ().

FIGS. 5(A-B) illustrate (A) OAS1 expression () over 15 days in plasma samples of a patient treated with 300 mg t.i.d. of SCY-635 over a 15 day period, and (▪) log change in HCV from baseline over the same period; and, (B) a comparison of plasma SCY-635 concentration (▪) over the study period and OAS1 expression ().

FIGS. 6(A-B) illustrate (A) OAS1 expression () over 15 days in plasma samples of a patient treated with 300 mg t.i.d. of SCY-635 over a 15 day period, and (▪) log change in HCV from baseline over the same period; and, (B) a comparison of plasma SCY-635 concentration (▪) over the study period and OAS1 expression ().

FIG. 7 illustrates OAS1 expression () over 15 days in plasma samples of a patient treated with placebo over a 15 day period, and (▪) log change in HCV from baseline over the same period.

FIGS. 8 (A-B) illustrate a comparison of IFN-α responses following treatment with SCY-635 in Subject 0072 (TT IL28B Genotype) and Subject 0071 (CC IL28B Genotype).

FIGS. 9 (A-B) illustrate a comparison of IFN-λ1 (IL-29) responses following treatment with SCY-635 in Subject 0072 (TT IL28B Genotype) and Subject 0071 (CC IL28B Genotype)

FIGS. 10 (A-B) illustrate a comparison of IFN-λ3 (IL-28B) responses following treatment with SCY-635 in Subject 0072 (TT IL28B Genotype) and Subject 0071 (CC IL28B Genotype)

FIGS. 11 (A-B) illustrate a comparison of IFN-β responses following treatment with SCY-635 in Subject 0072 (TT IL28B Genotype) and Subject 0071 (CC IL28B Genotype)

FIG. 12 illustrates the maximum_{log10} decrease in HCV RNA from baseline versus the fold change in 2′5′OAS concentration.

FIG. 13 illustrates the maximum_{log10} decrease in HCV RNA from baseline versus the fold change in neopterin concentration.

FIG. 14 illustrates the plasma concentrations of SCY-635 and OAS following treatments with a placebo.

FIG. 15 illustrates the plasma concentrations of SCY-635 and OAS following treatments with SCY-635 in Subject 0072.

FIG. 16 illustrates the plasma concentrations of SCY-635 and OAS following treatments with SCY-635 in Subject 0069.

FIG. 17 illustrates the plasma concentrations of SCY-635 and OAS following treatments with SCY-635 in Subject 0067.

FIG. 18 illustrates the plasma concentrations of SCY-635 and OAS following treatments with SCY-635 in Subject 0073.

FIG. 19 illustrates the plasma concentrations of SCY-635 and OAS following treatments with SCY-635 in Subject 0070.

FIG. 20 illustrates the plasma concentrations of SCY-635 and OAS following treatments with SCY-635 in Subject 0071.

FIGS. 21 (A-B) illustrate a comparison of plasma SCY-635 concentration (▪) over the study period and interferon alpha expression () over 14 days in plasma samples of patients (subjects 1001 and 0021) treated with 500 mg b.i.d. of SCY-635 over a 14 day period.

FIGS. 22 (A-C) illustrate a comparison of plasma SCY-635 concentration (▪) over the study period and interferon beta expression () over 14 days in plasma samples of patients (subjects 0022, 1017 and 0021) treated with 500 mg b.i.d. of SCY-635 over a 14 day period.

FIG. 23 illustrates a comparison of plasma SCY-635 concentration (▪) over the study period and interferon Lambda 1 (IL29) expression () over 14 days in plasma samples of a patient (subject 0021) treated with 500 mg b.i.d. of SCY-635 over a 14 day period.

FIG. 24 illustrates the induction of Interferon alpha in subgenomic con1b HuH7 and parental HuH7 cells following incubation with SCY-635 and IFNα2-b.

FIG. 25A illustrates the induction of 2′-5′ OAS in subgenomic con1b HuH7 and parental HuH7 cells following incubation with SCY-635.

FIG. 25B illustrates the induction of interferon lambda in subgenomic con1b HuH7 cells following incubation with SCY-635.

FIG. 26 illustrates the group mean dose response for Cohorts 4, 5 and 6.

FIG. 27 illustrates the individual viral load response for subjects in Cohort 6.

FIGS. 28 (A-C), 29 (A-C), 30 (A-C), 31 (A-C) and 32 (A-C) illustrate that interferon and 2′5′OAS-1 production is dependent upon the dose of SCY-635 and whether there is an HCV infection.

FIGS. 33 (A-E), 34 (A-E), 35 (A-E), 36 (A-E), 37 (A-E) and 38 (A-E) illustrate the correlation between SCY-635 plasma levels and the expression of Type 1 and Type 3 interferons and 2′5′OAS-1 in Cohort 6 individuals.

### DETAILED DESCRIPTION

### Definitions

“Cyclophilin-binding compound” is a compound capable of binding to one or more cyclophilins. For example, exemplary compounds can include cyclosporines which are useful in the treatment of certain indications and exhibit beneficial properties such as, for example, properties like those exhibited by exemplary compound SCY-635 and other exemplary cyclosporines. Such beneficial properties include, for example, interferon-like behavior.

“Cyclosporine” refers to any cyclosporine compound known to those of skill in the art, or a derivative thereof. See, e.g., Ruegger et al., 1976, Helv. Chim. Acta. 59:1075-92; Borel et al., 1977, Immunology 32:1017-25; the contents of which are hereby incorporated by reference in their entireties. Cyclosporine compounds include cyclosporine derivatives. A cyclosporine described herein may be cyclosporine A, and a cyclosporine derivative described herein may be a derivative of cyclosporine A.

“Polymorphic site” or “PS” refers to the position in a genetic locus or gene at which a polymorphism is found, e.g., single nucleotide polymorphism (SNP), restriction fragment length polymorphism (RFLP), variable number of tandem repeat (VNTR), dinucleotide repeat, trinucleotide repeat, tetranucleotide repeat, simple sequence repeat, insertion element such as AIu, and deletion or insertion of one or more nucleotides. A PS is usually preceded by and followed by highly conserved sequences in the population of interest and thus the location of a PS is typically made in reference to a consensus nucleic acid sequence of thirty to sixty nucleotides that bracket the PS, which in the case of a SNP is commonly referred to as the “SNP context sequence.” The location of the PS can also be identified by its location in a consensus or reference sequence relative to the initiation codon (ATG) for protein translation. The skilled artisan understands that the location of a particular PS may not occur at precisely the same position in a reference or context sequence in each individual in a population of interest due to the presence of one or more insertions or deletions in that individual as compared to the consensus or reference sequence. Moreover, it is routine for the skilled artisan to design robust, specific and accurate assays for detecting the alternative alleles at a polymorphic site in any given individual, when the skilled artisan is provided with the identity of the alternative alleles at the PS to be detected and one or both of a reference sequence or context sequence in which the PS occurs. Thus, the skilled artisan will understand that specifying the location of any PS described herein by reference to a particular position in a reference or context sequence (or with respect to an initiation codon in such a sequence) is merely for convenience and that any specifically enumerated nucleotide position literally includes whatever nucleotide position the same PS is actually located at in the same locus in any individual being tested for the presence or absence of a genetic marker using any of the genotyping methods described herein or other genotyping methods well-known in the art.

The term “derivative” refers to changes in the recited molecule that do not affect the basic structure of the molecule. For example, cyclosporin A is a cyclic nonribosomal peptide of 11 amino acids and contains a single D-amino acid.

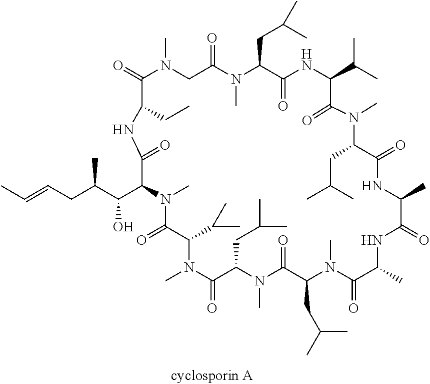

The term “derivative of cyclosporin A” encompasses compounds in which the characteristic structure of cyclosporin A has been modified in one or more portions to provide another compound which has cyclophilin-binding properties.

Sanglifehrin A consists of a 22-membered macrocycle, bearing in position 23 a nine-carbon tether terminated by a highly substituted spirobicyclic moiety.

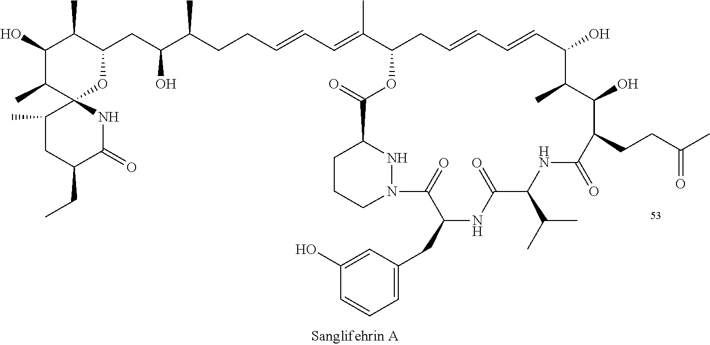

The term derivative of Sanglifehrin A encompasses compounds in which the 22-membered macrocycle, bearing in position 23 a nine-carbon tether terminated by a highly substituted spirobicyclic moiety is present in the molecule.

The cyclosporine nomenclature and numbering systems used hereafter are those used by J. Kallen et al., “Cyclosporins: Recent Developments in Biosynthesis, Pharmacology and Biology, and Clinical Applications”, Biotechnology, second edition, H.-J. Rehm and G. Reed, ed., 1997, p 535-591 and are shown below:

| Position | Amino acid in cyclosporine A |
|---|---|
| 1 | N-Methyl-butenyl-threonine (MeBmt) |
| 2 | [alpha]-aminobutyric acid (Abu) |
| 3 | Sarcosine (Sar) |
| 4 | N-Methyl-leucine (MeLeu) |
| 5 | Valine (Val) |
| 6 | N-Methyl-leucine (MeLeu) |
| 7 | Alanine (Ala) |
| 8 | (D)-Alanine ((D)-Ala) |
| 9 | N-Methyl-leucine (Me-Leu) |
| 10 | N-Methyl-leucine (MeLeu) |
| 11 | N-Methylvaline (MeVal) |

Thus, in one aspect, the cyclophilin inhibitor is a compound of general formula (I):

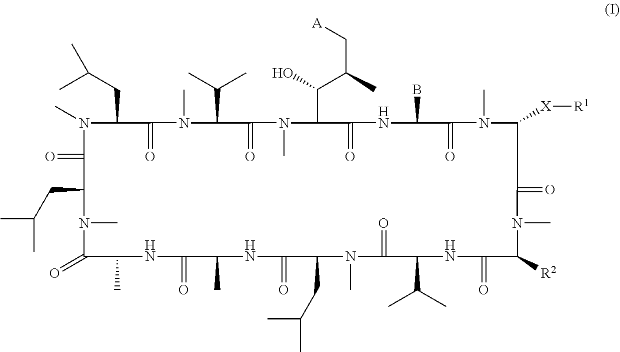

wherein:
A is (E) —CH═CHCH₃;
B is ethyl, 1-hydroxyethyl, isopropyl, or n-propyl;
R′ is:
   straight- or branched-chain alkyl containing from one to six carbon atoms, optionally substituted by one or more groups R¹³ which may be the same or different; straight- or branched-chain alkenyl containing from two to six carbon atoms optionally substituted by one or more groups which may be the same or different selected from the group consisting of halogen, hydroxy, amino, monoalkylamino and dialkylamino; straight- or branched-chain alkynyl containing from two to six carbon atoms, optionally substituted by one or one or more groups which may be the same or different selected from the group consisting of halogen, hydroxy, amino, monoalkylamino and dialkylamino;
   cycloalkyl containing from three to six carbon atoms optionally substituted by one or more groups which may be the same or different selected from the group consisting of halogen, hydroxy, amino, monoalkylamino and dialkylamino;
   straight- or branched-chain alkoxycarbonyl containing from one to six carbon atoms;
R¹² is isobutyl or 2-hydroxyisobutyl;
X is sulfur or oxygen;
R¹³ is selected from the group consisting of halogen, hydroxy, carboxyl, alkoxycarbonyl, —NR¹⁴R¹⁵ and —NR¹⁵(CH₂)ₘ₁NR¹⁴R¹⁵;
each R¹⁴ and R¹⁵, which may be the same or different, is independently:
   hydrogen;
   straight- or branched-chain alkyl comprising from one to six carbon atoms, optionally substituted by one or more groups R¹⁷ which may be the same or different;
   straight- or branched-chain alkenyl or alkynyl comprising from two to four carbon atoms;
   cycloalkyl containing from three to six carbon atoms optionally substituted by straight- or branched-chain alkyl containing from one to six carbon atoms;
   phenyl optionally substituted by from one to five groups which may be the same or different selected from the group consisting of halogen, alkoxy, alkoxycarbonyl, amino, alkylamino and dialkylamino;
   a heterocyclic ring which may be saturated or unsaturated containing five or six ring atoms and from one to three heteroatoms which may the same or different selected from nitrogen, sulfur and oxygen;
or R¹⁴ and R¹⁵, together with the nitrogen atom to which they are attached, form a saturated or unsaturated heterocyclic ring containing from four to six ring atoms, which ring may optionally contain another heteroatom selected from the group consisting of nitrogen, oxygen and sulfur and may be optionally substituted by from one to four groups which may be the same or different selected from the group consisting of alkyl, phenyl and benzyl;
R¹⁶ is hydrogen or straight- or branched-chain alkyl containing from one to six carbon atoms;
R¹⁷ is selected from the group consisting of halogen, hydroxy, carboxyl, alkoxycarbonyl and —NR¹⁸R¹⁹;
R¹⁸ and R¹⁹, which may be the same or different, each represent hydrogen or straight- or branched-chain alkyl containing from one to six carbon atoms;
and m1 is an integer from two to four;
   or a pharmaceutically acceptable salt or solvate thereof.

In a further embodiment, the cyclophilin inhibitor is a compound of general formula (I) above in which:
A is (E) —CH═CHCH₃;
B is ethyl, 1-hydroxyethyl, isopropyl or n-propyl;
R¹ represents —Y²—Ar2;
R² represents isobutyl or 2-hydroxyisobutyl;
X represents sulfur or oxygen;
Y² represents straight- or branched-C₁₋₆ alkylene, C₂₋₆ alkenylene or C₂₋₆ alkynylene;
Ar2 represents:
   phenyl optionally substituted by from one to five groups R²³ which may be the same or different;
   or a heterocyclic ring optionally substituted by one or more groups R²³ which may be the same or different, wherein said heterocyclic ring is attached to the group Y¹ via a ring carbon atom;
R²³ is selected from the group consisting of halogen, hydroxy, C₁₋₆ alkyl, C₁₋₆ alkoxy, C₁₋₆ haloalkyl, C₁₋₆ haloalkoxy, carboxyl, alkoxycarbonyl, —NR²⁴R²⁵ and —NR²⁶(CH₂)ₘ₂NR²⁴R²⁵;
R²⁴ and R²⁵, which may be the same or different, each represent:—
   hydrogen;
   straight- or branched-chain alkyl comprising from one to six carbon atoms, optionally substituted by one or more halogen;
   straight- or branched-chain alkenyl or alkynyl comprising from two to four carbon atoms;
   cycloalkyl containing from three to six carbon atoms optionally substituted by straight- or branched-chain alkyl containing from one to six carbon atoms;
or R²⁴ and R²⁵, together with the nitrogen atom to which they are attached, form a saturated or unsaturated heterocyclic ring containing from four to six ring atoms, which ring may optionally contain another heteroatom selected from the group consisting of nitrogen, oxygen and sulfur and may be optionally substituted by from one to four groups which may be the same or different selected from the group consisting of alkyl, phenyl and benzyl;
R²⁶ represents hydrogen or straight- or branched-chain alkyl containing from one to six carbon atoms;
m2 is an integer from two to four;
or a pharmaceutically acceptable salt thereof.

In one embodiment there is provided a pharmaceutical composition comprising a cyclophilin-binding compound for treating a patient having a disease susceptible to treatment with the cyclophilin-binding compound and a positive test for at least one cyclophilin-binding compound marker, wherein the cyclophilin-binding compound marker is a polymorphism residing about 3 kilobases (kb) upstream of the IL28B gene, encoding interferon-lambda-3. In a further embodiment the cyclophilin-binding compound marker is a C/T polymorphism, identified as rs 12979860 in the NCBI SNP Database.

An individual to be tested in, or treated by, any of the methods and products described herein is a subject in need of treatment with a cyclophilin-binding compound, preferably a human subject. In some embodiments, the individual has been diagnosed with, or exhibits a symptom of, a disease susceptible to treatment with a cyclophilin-binding compound. In other embodiments, the cyclophilin-binding compound drug to be used has been approved for use in treating an indication with which the individual has been diagnosed. In yet other embodiments, the cyclophilin-binding compound drug to be used is not approved for treating the diagnosed disease or exhibited symptom(s), but the prescribing physician believes the drug may be helpful in treating the individual.

The cyclophilin-binding compound used in the pharmaceutical compositions, drug products and methods can be any of the pharmaceutical compositions comprising an effective amount of a compound capable of binding cyclophilin, or a pharmaceutically acceptable salt, solvate or hydrate thereof, particularly a therapeutically effective amount, that is, an amount effective to prevent or substantially inhibit the development of, or to alleviate the existing symptoms of the subject being treated, in particular symptoms of a disease caused by a cell proliferation disorder, especially a viral infection or cancer. Determining the effective amount or the therapeutically effective amount is well within the capability of those skilled in the art, especially in light of the detailed disclosure provided herein. Dose ranges that can be used in determining an effective/therapeutically effective amount are provided below. A preferred example of a cyclophilin-binding compound/cyclophilin inhibitor is SCY-635, or a pharmaceutically acceptable salt, solvate or hydrate thereof.

Alternative cyclophilin-binding compounds include cyclosporine A; a derivative of cyclosporine A; sanglifehrin A or a derivative of sanglifehrin A. The cyclophilin-binding compound can be selected from the group consisting of cyclosporine A, SCY-635, alisporivir and NIM-811. In other examples, the cyclophilin-binding compound can be a cyclosporine A derivative as described in any one of International Patent Publication Nos. WO2005/021028, WO2006/039668, WO2006/038088, WO2007/041631, WO2008/069917, WO2010/002428, WO2010/076329, WO2010/088573, the contents of which are hereby incorporated by reference in their entireties and relied upon. Examples of sanglifehrin A derivatives are sangamides described in Moss et al., Med Chem Comm 2011 (DOI: 10.1039/C1MD00227A) and International Patent Publication No. WO2011/144924, the contents of which are hereby incorporated by reference in their entireties and relied upon.

A method of treating a human subject infected with hepatitis C virus can comprise administering an effective amount of a cyclophilin-binding compound, wherein a genotype of the subject includes the presence in the subject of a polymorphism residing in a cyclophilin-binding compound marker. In some embodiments, the polymorphism may be selected from those listed in Table 1.

The polymorphism may be located about 3 kilobases (kb) upstream of the IL28B gene, encoding interferon-lambda-3. In one example, the polymorphism is a C/T polymorphism, identified in the SNP rs12979860 in the NCBI SNP Database allele of the IL28b gene. Without being bound by any particular theory, the inventors believe that a better response allele can refer to an allele that provides a more robust response to a cyclophilin-binding compound.

TABLE 1
Cyclophilin-binding compound markers

| | | | | Homozygous |
|---|---|---|---|---|
| | | Better | Heterozygous | cyclophilin- |
| | | Response | cyclophilin-binding | binding |
| PS | SNP | Allele | compound marker | compound marker |
| rs12979860 | C/T | C | C/T genotype | C/C genotype |
| rs28416813 | G/C | G | G/C genotype | G/G genotype |
| rs8103142 | A/G | A | A/G genotype | A/A genotype |
| rs12980275 | A/G | A | A/G genotype | A/A genotype |
| rs8099917 | A/C | A | A/C genotype | A/A genotype |
| rs12972991 | T/G | T | T/G genotype | T/T genotype |
| rs8109886 | A/C | C | C/A genotype | C/C genotype |
| rs4803223 | T/C | T | T/C genotype | T/T genotype |
| rs12980602 | A/G | A | A/G genotype | A/A genotype |

Diseases and conditions that can be treated in accordance with the methods described herein are generally those that are susceptible to treatment with a cyclophilin-binding compound, i.e., the cyclophilin-binding compound achieves a clinically measurable beneficial result in a group of patients with the disease, e.g., reduction in viral load in HCV-infected patients. Exemplary diseases and conditions susceptible to treatment with a cyclophilin-binding compound include but are not limited to diseases caused by cell proliferation disorders, in particular viral infections, and cancers. Preferably, the disease is one for which the cyclophilin-binding compound has been approved by a regulatory agency such as the U.S. Food and Drug Administration. Viral infections include hepatitis A, hepatitis B, hepatitis C, hepatitis D, other non-A/non-B hepatitis, herpes virus, Epstein-Barr virus (EBV), cytomegalovirus (CMV), herpes simplex, human herpes virus type 6, papilloma, poxvirus, picornavirus, adenovirus, rhinoviral, human T lymphotropic virus-type 1 and 2, human rotavirus, rabies, retroviruses including human immunodeficiency virus (HIV), encephalitis and respiratory viral infections. Cancers include melanoma, chronic myelogenous leukemia (CML), renal cell cancer (RCC), hairy cell leukemia, Kaposi's sarcoma, multiple myeloma, basal cell carcinoma, malignant melanoma, superficial bladder cancer (SBC), ovarian cancer, follicular lymphoma, non-Hodgkin's lymphoma, cutaneous T cell lymphoma, condyloma accuminata, mycosis fungoides, carcinoid syndrome, colorectal cancer, laryngeal papillomatosis, and actinic keratosis. The types of viral infections and cancers that can be treated are not limited to those listed above. In preferred embodiments, the viral infection is HCV or HBV. In a particularly preferred embodiment, the viral infection is chronic HCV infection.

In another method there is provided an assay for evaluating the likelihood that a patient will respond to treatment by a cyclophilin-binding compound, said method comprising: (a) determining in a sample taken from patient an IL28B gene polymorphism residing about 3 kilobases (kb) upstream of the IL28B gene, encoding interferon-lambda-3; (b) generating an efficacy index based upon one or more polymorphisms of the gene; and (c) evaluating the likelihood that said subject will respond to the cyclophilin-binding compound based upon said efficacy index. In certain embodiments, the presence in the patient of a marker genotype shown in Table 1 indicates the patient has an increased likelihood of responding to treatment as compared to a marker genotype not shown in Table 1. In one aspect of this embodiment the patient is infected with a viral disease, such as HCV.

In some examples of such a method, an efficacy index can be compared to an index cutoff value. An efficacy index can be obtained by correlating one or more parameters from each of a plurality of patients with their individual responses to treatment, and determining a value related to the success in treatment of a patient having a specific condition. The index cut-off value is a value that can be used to determine the likelihood of whether a subject having a specific efficacy index will respond to treatment. An index cut-off value can be obtained by correlating the efficacy index from each of a plurality of subjects with their individual responses to treatment, and determining a value, or a set of statistical values, relating to the probability of successful treatment of a specific condition. Procedures that can be used to accomplish this are known to those in the art. In some examples of such a method, an efficacy index greater than said index cutoff value indicates that the subject does not have a high likelihood of responding to the cyclophilin-binding compound. In some examples of such a method, the sample can be selected from the group consisting of whole blood, serum, plasma, buccal cells, combinations thereof and the like.

The presence or absence of a cyclophilin-binding compound marker can be detected by any of a variety of genotyping techniques commonly used in the art. Typically, such genotyping techniques employ one or more oligonucleotides that are complementary to a region containing, or adjacent to, the PS of interest. The sequence of an oligonucleotide used for genotyping a particular PS of interest is typically designed based on a context sequence for the PS.

The location, in a particular individual, of any of the polymorphic sites identified in Table 1 is at a position corresponding to the location of the PS of interest in a reference coding or genomic DNA sequence surrounding the PS or interest or in one of the context sequences described in Table 2 below, or their complementary sequences. Longer context sequences useful in designing oligonucleotides to genotype the PS of Table 1 are the context sequences listed in the NCBI SNP Database as of May 19, 2009. Reference coding and amino acid sequences for IFN-[lambda]3 are those shown in GenBank Accession No. AY 129149 (Version Y129149.1, GI:25527104) in the NCBI Nucleotide database on May 19, 2009.

TABLE 2
Context sequences for SNPs associated with
Cyclophilin-binding compound.

| | | Sequence |
|---|---|---|
| | | ID |
| PS | Short Context Sequence | No. |
| rs12979860 | CTGAACCAGGGAGCTCCCCGAAGGCG | 1 |
| | YGAACCAGGGTTGAATTGCACTCCGC | |
| rs28416813 | CAGAGAGAAAGGGAGCTGAGGGAATG | 2 |
| | SAGAGGCTGCCCACTGAGGGCAGGGG | |
| rs8103142 | TCCTGGGGAAGAGGCGGGAGCGGCAC | 3 |
| | YTGCAGTCCTTCAGCAGAAGCGACTC | |
| rs12980275 | CTGAGAGAAGTCAAATTCCTAGAAAC | 4 |
| | RGACGTGTCTAAATATTTGCCGGGGT | |
| rs8099917 | CTTTTGTTTTCCTTTCTGTGAGCAAT | 5 |
| | KTCACCCAAATTGGAACCATGCTGTA | |
| rs12972991 | AGAACAAATGCTGTATGATTCCCCCT | 6 |
| | MCATGAGGTGCTGAGAGAAGTCAAAT | |
| rs8109886 | TATTCATTTTTCCAACAAGCATCCTG | 7 |
| | MCCCAGGTCGCTCTGTCTGTCTCAAT | |
| rs4803223 | CCTAAATATGATTTCCTAAATCATAC | 8 |
| | RGACATATTTCCTTGGGAGCTATACA | |
| rs12980602 | TCATATAACAATATGAAAGCCAGAGA | 9 |
| | YAGCTCGTCTGAGACACAGATGAACA | |

Context sequences reported in NCB1 SNP Database on May 20, 2009; Y indicates C or T, S indicates G or C, R indicates G or A, K = G or T, M = A or C.

As recognized by the skilled artisan, nucleic acid samples containing a particular PS can be complementary double stranded molecules and thus reference to a particular site on the sense strand refers as well to the corresponding site on the complementary antisense strand. Similarly, reference to a particular genotype obtained for a PS on both copies of one strand of a chromosome is equivalent to the complementary genotype obtained for the same PS on both copies of the other strand. Thus, an A/A genotype for the rs8103142 PS on the coding strand for the IL28B gene is equivalent to a T/T genotype for that PS on the noncoding strand.

The context sequences recited herein, as well as their complementary sequences, can be used to design probes and primers for genotyping the polymorphic sites of Table 1 in a nucleic acid sample obtained from a human subject of interest using any of a variety of methods well known in the art that permits the determination of whether the individual is heterozygous or homozygous for the better response allele identified in Table 1. Nucleic acid molecules utilized in such methods generally include RNA, genomic DNA, or cDNA derived from RNA.

Typically, genotyping methods involve assaying a nucleic acid sample prepared from a biological sample obtained from the individual to determine the identity of a nucleotide or nucleotide pair present at one or more polymorphic sites of interest. Nucleic acid samples can be prepared from virtually any biological sample. For example, convenient samples include whole blood serum, semen, saliva, tears, fecal matter, urine, sweat, buccal matter, skin and hair. Somatic cells are preferred since they allow the determination of the identity of both alleles present at the PS of interest.

Nucleic acid samples can be prepared for analysis using any technique known to those skilled in the art. Preferably, such techniques result in the isolation of genomic DNA sufficiently pure for determining the genotype for the desired polymorphic site(s) in the nucleic acid molecule. To enhance the sensitivity and specificity of that determination, it is frequently desirable to amplify from the nucleic acid sample a target region containing the PS to be genotyped. Nucleic acid isolation and amplification techniques can be found, for example, in Sambrook, et al., Molecular Cloning: A Laboratory Manual (Cold Spring Harbor Laboratory, New York) (2001).

Any amplification technique known to those of skill in the art can be used including, but not limited to, polymerase chain reaction (PCR) techniques. PCR can be carried out using materials and methods known to those of skill in the art (See generally PCR Technology: Principals and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Matilla et al., Nucleic Acids Res. 19: 4967 (1991); Eckert et al., PCR Methods and Applications 1: 17 (1991); PCR (eds. McPherson et al., IRL Press, Oxford); and U.S. Pat. No. 4,683,202. Other suitable amplification methods include the ligase chain reaction (LCR) (see Wu and Wallace, Genomics 4: 560 (1989) and Landegren et al., Science 241: 1077 (1988)), transcription amplification (Kwoh et al., Proc. Natl. Acad. Sci. USA 86: 1 173 (1989)), self-sustained sequence replication (Guatelli et al., Proc. Nat. Acad. Sci. USA, 87: 1874 (1990)); isothermal methods (Walker et al., Proc. Natl. Acad. Sci. USA 89:392-6 (1992)); and nucleic acid-based sequence amplification (NASBA).

The amplified target region is assayed to determine the identity of at least one of the alleles present at a PS in the target region. If both alleles of a locus are represented in the amplified target, it will be readily appreciated by the skilled artisan that only one allele will be detected at a PS in individuals who are homozygous at that PS, while two different alleles will be detected if the individual is heterozygous for that PS.

The identity of the allele can be identified directly, known as positive-type identification, or by inference, referred to as negative-type identification. For example, where a SNP is known to be guanine or cytosine in a reference population, a PS can be positively determined to be either guanine or cytosine for an individual homozygous at that site, or both guanine and cytosine, if the individual is heterozygous at that site. Alternatively, the PS can be negatively determined to be not guanine (and thus cytosine/cytosine) or not cytosine (and thus guanine/guanine).

Identifying the allele or pair of alleles (e.g., the two nucleotides in case of a SNP) at a PS in a nucleic acid sample obtained from an individual can be accomplished using any technique known to those of skill in the art. Preferred techniques permit rapid, accurate assaying of multiple PS with a minimum of sample handling. Some examples of suitable techniques include, but are not limited to, direct DNA sequencing of the amplified target region, capillary electrophoresis, hybridization of allele-specific probes, single-strand conformation polymorphism analysis, denaturing gradient gel electrophoresis, temperature gradient electrophoresis, mismatch detection; nucleic acid arrays, primer specific extension, protein detection, and other techniques well known in the art. See, for example, Sambrook, et al., Molecular Cloning: A Laboratory Manual (Cold Spring Harbor Laboratory, New York) (2001); Ausubel, et al., Current Protocols in Molecular Biology (John Wiley and Sons, New York) (1997); Orita et al., Proc. Nat. Acad. ScL USA 86, 2766-2770 (1989); Humphries et al., in MOLECULAR DIAGNOSIS OF GENETIC DISEASES, Elles, ed., pp. 32 1-340, 1996; Wartell et al., Nucl. Acids Res. 18:2699-706 (1990); Hsu et al. (1994) Carcinogenesis 15: 1657-1662; Sheffield et al., Proc. Natl. Acad. ScL USA 86:232-6 (1989); Winter et al., Proc. Natl. Acad. Sci. USA 82:7575 (1985); Myers et al. (1985) Nature 313:495; Rosenbaum and Reissner (1987) Biophys Chem. 265: 12753; Modrich, Ann. Rev. Genet. 25:229-53 (1991); U.S. Pat. No. 6,300,063; U.S. Pat. No. 5,837,832; U.S. Pat. No. 5,459,039; and HuSNP Mapping Assay, reagent kit and user manual, Affymetrix Part No. 90094 (Affymetrix, Santa Clara, Calif.).

In preferred embodiments, the identity of the allele(s) at a PS is determined using a polymerase-mediated primer extension method. Several such methods have been described in the patent and scientific literature and include the “Genetic Bit Analysis” method (WO 92/15712) and the ligase/polymerase mediated genetic bit analysis (U.S. Pat. No. 5,679,524. Related methods are disclosed in WO 9 1/02087, WO 90/09455, WO 95/17676, and U.S. Pat. Nos. 5,302,509 and 5,945,283. Extended primers containing the complement of the polymorphism can be detected by mass spectrometry as described in U.S. Pat. No. 5,605,798.

Another primer extension method employs allele specific PCR (Ruano, G. et al., Nucl. Acids Res. 17:8392 (1989); Ruano, G. et al., Nucl. Acids Res. 19:6877-82 (1991); WO 93/22456; Turki et al., J. Gun. Invest. 95:1635-41 (1995)). In addition, multiple PSs can be investigated by simultaneously amplifying multiple regions of the nucleic acid using sets of allele-specific primers as described in WO 89/10414.

Yet another primer extension method for identifying and analyzing polymorphisms utilizes single-base extension (SBE) of a fluorescently-labeled primer coupled with fluorescence resonance energy transfer (FRET) between the label of the added base and the label of the primer. Typically, the method, such as that described by Chen et al., P roc. Nat. Acad. Sci. 94:10756-61 (1997) uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

A preferred genotyping assay is a TaqMan(R)SNP Genotyping Assay from Applied Biosystems or an assay having about the same reliability, accuracy and specificity.

In all of the above methods, the accuracy and specificity of an assay designed to detect the identity of the allele(s) at any PS is typically validated by performing the assay on DNA samples in which the identity of the allele(s) at that PS is known. Preferably, a sample representing each possible allele is included in the validation process. For diploid loci such as those on autosomal and X chromosomes, the validation samples will typically include a sample that is homozygous for the major allele at the PS, a sample that is homozygous for the minor allele at the PS, and a sample that is heterozygous at that PS. These validation samples are typically also included as controls when performing the assay on a test sample (i.e., a sample in which the identity of the allele(s) at the PS is unknown). The specificity of an assay can also be confirmed by comparing the assay result for a test sample with the result obtained for the same sample using a different type of assay, such as by determining the sequence of an amplified target region believed to contain the PS of interest and comparing the determined sequence to context sequences accepted in the art, such as the context sequences provided herein.

The length of the context sequence necessary to establish that the correct genomic position is being assayed will vary based on the uniqueness of the sequence in the target region (for example, there may be one or more highly homologous sequences located in other genomic regions). The skilled artisan can readily determine an appropriate length for a context sequence for any PS using known techniques such as blasting the context sequence against publicly available sequence databases. For amplified target regions, which provide a first level of specificity, examining the context sequence of about 30 to 60 bases on each side of the PS in known samples is typically sufficient to ensure that the assay design is specific for the PS of interest. Occasionally, a validated assay may fail to provide an unambiguous result for a test sample. This is usually the result of the sample having DNA of insufficient purity or quantity, and an unambiguous result is usually obtained by repurifying or reisolating the DNA sample or by assaying the sample using a different type of assay.

Further, in performing any of the methods described herein that require determining the presence or absence of a particular cyclophilin-binding compound marker, such determination can be made by consulting a data repository that contains sufficient information on the patient's genetic composition to determine whether the patient has the marker of interest. Preferably, the data repository lists what cyclophilin-binding compound marker(s) are present and absent in the individual. The data repository could include the individual's patient records, a medical data card, a file (e.g., a flat ASCII file) accessible by a computer or other electronic or non-electronic media on which appropriate information or genetic data can be stored. As used herein, a medical data card is a portable storage device such as a magnetic data card, a smart card, which has an on-board processing unit and which is sold by vendors such as Siemens of Munich Germany, or a flash-memory card. If the data repository is a file accessible by a computer; such files may be located on various media, including: a server, a client, a hard disk, a CD, a DVD, a personal digital assistant such as a Palm Pilot a tape, a zip disk, the computer's internal ROM (read-only-memory) or the internet or worldwide web. Other media for the storage of files accessible by a computer will be obvious to one skilled in the art.

Testing for a cyclophilin-binding compound marker can be conducted by determining whether the individual has an allele, e.g., nucleotide, at a different locus that is in high linkage disequilibrium (LD) with the better response allele for any of the SNPs listed in Table 1. Two particular alleles at different loci on the same chromosome are said to be in LD if the presence of one of the alleles at one locus tends to predict the presence of the other allele at the other locus. Such variants, which are referred to herein as linked variants, or proxy variants, can be any type of variant (e.g., a SNP, insertion or deletion) that is in high LD with the better response allele of interest.

Linked variants are readily identified by determining the degree of linkage disequilibrium (LD) between the better response allele of any of the SNPs in Table 1 and a candidate linked allele at a polymorphic site located in the chromosomal region 19q13.13 or elsewhere on chromosome 19. The candidate linked variant can be an allele of a polymorphism that is currently known. Other candidate linked variants can be readily identified by the skilled artisan using any technique well-known in the art for discovering polymorphisms.

The degree of LD between a better response allele in Table 1 and a candidate linked variant can be determined using any LD measurement known in the art. LD patterns in genomic regions are readily determined empirically in appropriately chosen samples using various techniques known in the art for determining whether any two alleles (e.g., between nucleotides at different PSs) are in linkage disequilibrium (see, e.g., GENETIC DATA ANALYSIS II, Weir, Sineuer Associates, Inc. Publishers, Sunderland, Mass. 1996). The skilled artisan can readily select which method of determining LD will be best suited for a particular population sample size and genomic region. One of the most frequently used measures of linkage disequilibrium is r², which is calculated using the formula described by Devlin et al. (Genomics, 29(2):311-22 (1995)). r² is the measure of how well an allele X at a first locus predicts the occurrence of an allele Y at a second locus on the same chromosome. The measure only reaches 1.0 when the prediction is perfect (e.g. X if and only if Y).

Preferably, the locus of the linked variant is in a genomic region of about 100 kilobases, more preferably about 10 kb that spans any of the PS of Table 1. Other linked variants are those in which the LD with the better response allele has a r² value, as measured in a suitable reference population, of at least 0.75, more preferably at least 0.80, even more preferably at least 0.85 or at least 0.90, yet more preferably at least 0.95, and most preferably 1.0. The reference population used for this r² measurement can be the general population, a population using the cyclophilin-binding compound, a population diagnosed with a particular condition for which the cyclophilin-binding compound shows efficacy (such as chronic HCV infection) or a population whose members are self-identified as belonging to the same ethnic group, such as Caucasian, African American, Hispanic, Latino, Native American and the like, or any combination of these categories. Preferably the reference population reflects the genetic diversity of the population of patients to be treated with a cyclophilin-binding compound.

In some embodiments, a physician determines whether a patient has a cyclophilin-binding compound marker described herein by ordering a diagnostic test, which is designed to determine whether the patient has at least one better response allele at one or more of the polymorphic sites in Table 1. Preferably the test determines the identity of both alleles, i.e., the genotype, at this PS. In some embodiments, the testing laboratory will prepare a nucleic acid sample from a biological sample (such as a blood sample or buccal swab) obtained from the patient. In some embodiments, a blood sample from the patient is drawn by the physician or a member of the physician's staff, or by a technician at a diagnostic laboratory. In some embodiments, the patient is provided with a kit for taking a buccal swab from the inside of his or her cheek, which the patient then gives to the physician's staff member or sends directly to the diagnostic laboratory.

In some embodiments, the testing laboratory does not know the identity of the individual whose sample it is testing; i.e., the sample received by the laboratory is made anonymous in some manner before being sent to the laboratory. For example, the sample can be merely identified by a number or some other code (a “sample ID”) and the results of the diagnostic method can be reported to the party ordering the test using the sample ID. In preferred embodiments, the link between the identity of an individual and the individual's sample is known only to the individual or to the individual's physician.

In some embodiments, after the test results have been obtained, the testing laboratory generates a test report which indicates whether the better response allele is present or absent at the genotyped polymorphic site, and preferably indicates whether the patient is heterozygous or homozygous for the better response allele. In some embodiments, the test report is a written document prepared by the testing laboratory and sent to the patient or the patient's physician as a hard copy or via electronic mail. In other embodiments, the test report is generated by a computer program and displayed on a video monitor in the physician's office. The test report can also comprise an oral transmission of the test results directly to the patient or the patient's physician or an authorized employee in the physician's office. Similarly, the test report can comprise a record of the test results that the physician makes in the patient's file.

In one preferred embodiment, if the patient is homozygous for the better response allele, then the test report further indicates that the patient tested positive for a genetic marker associated with a likely response to treatment with a cyclophilin-binding compound, while if the individual is heterozygous for the better response allele or is homozygous for the other allele, then the test report further indicates that the patient tested negative for a genetic marker associated with a likely response to treatment with a cyclophilin-binding compound. In some embodiments, the test result will include a probability score for achieving a beneficial response to the cyclophilin-binding compound, which is derived from running a model that weights various patient parameters (e.g., age, gender, weight, race, test results for other pharmacogenetic markers for the cyclophilin-binding compound) and disease parameters (e.g., disease severity) that are associated with the cyclophilin-binding compound marker in the relevant disease population. The probability score can be obtained by correlating various parameters from each of a plurality of patients with their individual responses to treatment, and determining a score relating to the probability of successful treatment of a specific condition. Procedures that can be used to accomplish this are known to those in the art. The weight given to each parameter is based on its contribution relative to the other parameters in explaining the inter-individual variability of response to the cyclophilin-binding compound in the relevant disease population. The doctor can use this response probability score as a guide in selecting a therapy or treatment regimen for the patient. For example, for chronic HCV infection, patient parameters associated with achieving SVR include race and disease parameters include HCV genotype, baseline viral load, and degree of fibrosis.

Typically, the individual would be tested for the presence of a cyclophilin-binding compound marker prior to initiation of cyclophilin-binding compound therapy, but it is envisioned that such testing could be performed at any time after the individual is administered the first dose of a cyclophilin-binding compound. For example, the treating physician may be concerned that the patient has not responded adequately and desires to test the individual to determine whether continued treatment with the cyclophilin-binding compound is warranted. In some embodiments, a physician can determine whether or not an individual should be tested for a cyclophilin-binding compound marker. For example, the physician may be considering whether to prescribe for the patient a pharmaceutical product that is indicated for patients who test positive for the cyclophilin-binding compound marker. In embodiments where the patient has detectable serum HCV RNA and has received a liver transplant, the physician may decide to have a biopsy from the transplanted liver tested for a cyclophilin-binding compound marker to aid making treatment decisions for the patient.

In deciding how to use the cyclophilin-binding compound marker test results in treating any individual patient, the physician can also take into account other relevant circumstances, such as the disease or condition to be treated, the age, weight, gender, genetic background and race of the patient, including inputting a combination of these factors and the genetic marker test results into a model that helps guide the physician in choosing a therapy and/or treatment regimen with that therapy.

The rs12979860 C allele is also associated with a greater likelihood of natural clearance of HCV in patients with acute hepatitis C, which refers to the first 6 months after infection with HCV. Between 60% to 70% of infected people develop no symptoms during the acute phase. However, some patients have symptoms of acute hepatitis C infection, which include decreased appetite, fatigue, abdominal pain, jaundice, itching and flu-like symptoms, which lead to an early diagnosis. Other patients are diagnosed with acute hepatitis C due to monitoring for HCV infection after a known exposure to an infected source, such as a needlestick injury. The hepatitis C virus is usually detectable in the blood by PCR within one to three weeks after infection, and antibodies to the virus are generally detectable within 3 to 15 weeks.

Because up to 50% of patients can spontaneously clear the virus from their bodies during the acute phase, physicians have traditionally been reluctant to subject a patient diagnosed with acute hepatitis to the expense and side effects of antiviral therapy unless and until the patient progresses to a chronic HCV infection, i.e., an infection lasting more than 6 months. Determining the patient's genotype at the rs12979860 PS can be another factor the physician could consider in deciding whether to begin antiviral therapy or delay therapy for six months after diagnosis with acute HCV infection. If the patient's genotype is heterozygous or homozygous C, the physician may decide to delay therapy for six months. If the patient's genotype is homozygous T, the physician may decide that early antiviral therapy is warranted since the patient is less likely to spontaneously clear the virus.

The methods provided herein include the treatment, prevention and management of diseases while reducing or avoiding adverse or unwanted effects, e.g., toxicities or side effects. The compounds described herein, such as SCY-635, alisporivir, or NIM-811, can be administered by any conventional route, in particular orally, parenterally, rectally or by inhalation (e.g., in the form of aerosols). The preferred route of administration for the doses and dosing regimens described herein is oral.

In certain embodiments, SCY-635, alisporivir, or NIM-811 or a pharmaceutically acceptable salt, solvate or hydrate thereof can be administered according to the doses and dosing regimens described herein in combination with a one or more additional active agents (e.g., simultaneously or sequentially). In particular embodiments, SCY-635, alisporivir, or NIM-811, or a pharmaceutically acceptable salt, solvate or hydrate thereof can be administered according to the doses and dosing schedules described herein in combination with the one or more additional active agents. The administration of the additional active agent(s) can be topical, enteral (e.g. oral, duodenal, rectal), parenteral (e.g., intravenous, intraarterial, intramuscular, subcutaneous, intradermal or interaperitoneal) or intrathecal.

Pharmaceutical compositions and unit dosage forms comprising SCY-635, alisporivir, or NIM-811, or a pharmaceutically acceptable salt, solvate or hydrate thereof, are also provided herein. Individual dosage forms may be suitable for oral, mucosal (including sublingual, buccal, rectal, nasal, or vaginal) or parenteral (including subcutaneous, intramuscular, bolus injection, intraartcrial, or intravenous) administration. Preferred pharmaceutical compositions and single unit dosage forms are suitable for oral administration. A unit dosage form is a form in which a specific dose of a compound, or a mixture of compounds in specific amounts, is provided in which the entire content of the form is administered in a single administration.

Methods for therapy wherein a cyclophilin-binding compound such as SCY-635, alisporivir, or NIM-811, or a pharmaceutically acceptable salt, solvate or hydrate thereof, is administered to an infected human subject in need thereof can continue for a certain period of time (e.g., 5, 7, 10, 14, 20, 24, 28, 60, 120, 360 days or longer).

Methods for the administration of a cyclophilin-binding compound such as SCY-635, or a pharmaceutically acceptable salt, solvate or hydrate thereof, in divided doses (e.g., two or three times daily) of between about 4 mg/kg and about 50 mg/kg; between about 10 mg/kg and about 50 mg/kg; between about 10 mg/kg and about 34 mg/kg; between about 13 mg/kg and about 27 mg/kg; between about 14 mg/kg and about 20 mg/kg; between about 15 mg/kg and about 19 mg/kg; or between about 15 mg/kg and about 18 mg/kg, to a human subject infected with, or at risk for infection with, HCV. In another embodiment, any dose of a cyclophilin-binding compound such as SCY-635, or a pharmaceutically acceptable salt, solvate or hydrate, described in the preceding embodiment is administered two or three times in a 24 hour period.

An effective dose can be selected in accordance with genotyping criteria. For example, an efficacy index based upon a genotype can be compared to a dosing matrix in which various factors related to patients are used to select a dosage, interval, and duration of treatment. In a particular embodiment, a cyclophilin-binding compound such as SCY-635, or a pharmaceutically acceptable salt, solvate or hydrate thereof, can be administered in a dose of about 10 mg/kg, about 13 mg/kg, about 14 mg/kg, about 15 mg/kg, about 17 mg/kg, about 18 mg/kg or more or less, in accordance with an efficacy index, or the like.

As another example, dosages of a cyclophilin-binding compound such as SCY-635 (a) in an amount of about 200 mg each time, 3 times per day; (b) in an amount of about 250 mg each administration, 3 times per day; (c) in an amount of about 280 mg each administration, 3 times per day; (d) in an amount of about 300 mg each time, 3 times per day; (e) in an amount of about 330 mg each time, 3 times per day; (f) in an amount of about 350 mg each time, 3 times per day; (g) in an amount of about 400 mg each time, 3 times per day; (h) in an amount of about 500 mg each time, 3 times per day; or (i) in an amount of about 600 mg each time, 3 times per days can be selected in accordance with an efficacy index, or the like.

In one aspect of the above embodiments, a cyclophilin-binding compound such as SCY-635 is administered to a human subject once every 8 hours. In another aspect of the above embodiments, SCY-635 is administered at 7-, 7- and 10-hour intervals per day (e.g. at about 7:00 AM, about 2:00 PM, and at about 9:00 PM). An effective dosing schedule can be selected in accordance with a genotype of an efficacy index determined from a genotype.

Methods of treatment described herein can include administering to the human subject a cyclophilin-binding compound such as SCY-635 (a) in an amount of about 200 mg each time, 2 times per day, once every 12 hours; (b) in an amount of about 250 mg each time, 2 times per day, once every 12 hours; (c) in an amount of about 275 mg each time, 2 times per day, once every 12 hours; (d) in an amount of about 300 mg each time, 2 times per day, once every 12 hours; (e) in an amount of about 350 mg each time, 2 times per day, once every 12 hours; (f) in an amount of about 375 mg each time, 2 times per day, once every 12 hours; (g) in an amount of about 400 mg each time, 2 times per day, once every 12 hours; (h) in an amount of about 425 mg each time, 2 times per day, once every 12 hours; (i) in an amount of about 450 mg each time, 2 times per day, once every 12 hours; (j) in an amount of about 500 mg each time, 2 times per day, once every 12 hours; (k) in an amount of about 550 mg each time, 2 times per day, once every 12 hours; (l) in an amount of about 600 mg each time, 2 times per day, once every 12 hours; (m) in an amount of about 625 mg each time, 2 times per day, once every 12 hours; (n) in an amount of about 650 mg each time, 2 times per day, once every 12 hours; (o) in an amount of about 700 mg each time, 2 times per day, once every 12 hours; or (p) in an amount of about 800 mg each time, 2 times per day, once every 12 hours, wherein the dosing regimen is selected in accordance with an efficacy index, a genotype, or the like.

A method that provides greater than about 600 mg of a cyclophilin-binding compound such as SCY-635, given as a divided dose over a 24 hour period, can effectively result in a high trough level of a cyclophilin-binding compound such as SCY-635 in plasma. As used herein, “trough level” refers to the lowest level that a medicine is present in the body. It can be important, particularly in viral diseases, to maintain drug levels above a certain concentration to maintain appropriate inhibition of viral replication. In particular, it has been found that the a dosing regimen of greater than about 200 mg of a cyclophilin-binding compound such as a cyclophilin-binding compound such as SCY-635 each time, three times a day, once every 8 hours, can lead to disproportionately higher trough levels of a cyclophilin-binding compound such as SCY-635 than seen at lower daily doses.

A cyclophilin-binding compound such as SCY-635 can be given as a divided dose over a 24 hour period in a dosing regimen selected in accordance with a genotype analysis, efficacy index, or the like, which regimen includes administering to the human subject a cyclophilin-binding compound such as SCY-635 (a) in an amount of from 800 to 999 mg per day; (b) in an amount of from 810 to 997 mg per day; (c) in an amount of from 820 to 995 mg per day; (d) in an amount of from 850 to 950 mg per day; (e) in an amount of 870 to 930 mg per day; (f) in an amount of from 880 to 920 mg per day; or (g) in an amount of from 890 to 910 mg per day. In one aspect of these embodiments SCY-635 is given in two doses over a 24 hour period. In another aspect of these embodiments SCY-635 is given in three doses over a 24 hour period.

In another embodiment, a cyclophilin-binding compound such as SCY-635 can be given as a divided dose over a 24 hour period, which includes administering to the human subject a cyclophilin-binding compound such as SCY-635 (a) in an amount of from about 600 to about 1050 mg per day; (b) in an amount of from about 600 to about 1000 mg per day; (c) in an amount of from about 750 to about 1000 mg per day; (d) in an amount of from about 800 to about 1000 mg per day; or (e) in an amount of from about 900 to about 1000 mg per day. In one aspect of these embodiments SCY-635 is given in two doses over a 24 hour period. In another aspect of these embodiments SCY-635 is given in three doses over a 24 hour period.

A cyclophilin-binding compound such as SCY-635 can be given in two doses over a 24 hour period and the time between doses is from about 8 hours to about 16 hours. In another embodiment a cyclophilin-binding compound such as SCY-635 is given in two doses over a 24 hour period and the time between doses ranges from about 10 hours to about 14 hours.

A cyclophilin-binding compound such as SCY-635 is given in three doses over a 24 hour period and the time between doses is from about 4 hours to about 12 hours. In another embodiment a cyclophilin-binding compound such as SCY-635 is given in three doses over a 24 hour period and the time between doses ranges from about 6 hours to about 10 hours.

In another embodiment, a therapeutically effective plasma concentration of a cyclophilin-binding compound such as SCY-635 is obtained and a certain trough level concentration of SCY-635 is maintained at steady state. These methods can be particularly useful for treating a human infected with HCV by administering a cyclophilin-binding compound such as SCY-635 formulation, wherein a trough of a cyclophilin-binding compound such as SCY-635 plasma level is maintained at a minimum of about 110 ng/mL, about 115 ng/mL, about 135 ng/mL, about 216 ng/mL, or about 400 ng/mL, over a 24 hour period at steady state. In certain embodiments, the methods can be particularly useful for treating a human suffering from HCV infection by administering a cyclophilin-binding compound such as SCY-635, wherein the trough level of a cyclophilin-binding compound such as SCY-635 plasma level is maintained at a minimum of about 115 ng/mL over a 24 hour period at steady state. In certain embodiments, the methods can be particularly useful for treating, preventing or managing HCV infection in a human subject infected with, or at risk for infection with, HCV, wherein the compound is administered in amount sufficient to maintain a trough plasma concentration of the compound of greater than about 115 ng/ml at steady state.

A relatively rapid increase in plasma concentration can be obtained by administering a loading dose to a human subject. In one embodiment, the loading dose is about 400 mg of SCY-635. In another embodiment the loading dose is about 600 mg of a cyclophilin-binding compound such as SCY-635. In a further embodiment the loading dose is about 800 mg of SCY-635. In another embodiment the loading dose is about 900 mg of a cyclophilin-binding compound such as SCY-635. In another embodiment the loading dose is about 1000 mg of SCY-635. In a further embodiment, a loading dose of about 400 mg of a cyclophilin-binding compound such as SCY-635 is administered, followed by about 300 mg of a cyclophilin-binding compound such as SCY-635, administered two times a day. In a further embodiment, a loading dose of about 400 mg of a cyclophilin-binding compound such as SCY-635 is administered, followed by about 300 mg of SCY-635, administered three times a day.

In one embodiment, provided herein is a dosage form (other than the dosage form used to administer the loading dose) comprising about 300 mg of a cyclophilin-binding compound such as SCY-635, and the dosage form can be administered three times a day (e.g. t.i.d.). In other embodiments, the dosage form comprises about 300 mg of a cyclophilin-binding compound such as SCY-635 once every 8 hours (i.e. q8h).

In certain embodiments, the cyclophilin-binding compound such as SCY-635 dosage form can be administered once every 8 hours. In other embodiments, a cyclophilin-binding compound such as SCY-635 dosage form can be administered once every 7, 7 and 10 hours (e.g. at about 7:00 AM, about 2:00 PM, and at about 9:00 PM).

In certain embodiments, the treatment duration with SCY-635 is shorter than the current standard of care, for example where the genotype or efficacy index indicates susceptibility to treatment with a cyclophilin-binding compound such as SCY-635. In certain embodiments, a cyclophilin-binding compound such as SCY-635 is administered for less than about 182 days. In certain embodiments, a cyclophilin-binding compound such as SCY-635 is administered for about 91 days. In certain embodiments, a cyclophilin-binding compound such as SCY-635 is administered for about 28 days.

In another embodiment, provided herein are unit dosage formulations that comprise between about 600 mg and about 2000 mg, between about 800 mg and about 1600 mg, between about 850 mg and about 1200 mg, between about 850 mg and about 1100 mg, between about 900 mg and about 1100 mg, or between about 900 mg and about 1050 mg of a cyclophilin-binding compound such as SCY-635, or a pharmaceutically acceptable salt, solvate or hydrate thereof. In certain embodiments, provided herein are unit dosage formulations that comprise between about 800 mg and about 1600 mg of a cyclophilin-binding compound such as SCY-635, or a pharmaceutically acceptable salt, solvate or hydrate thereof.

In another embodiment, provided herein are unit dosage formulations that comprise about 100 mg, about 120 mg, about 150 mg, about 175 mg, about 200 mg, about 250 mg, about 280 mg, about 300 mg, about 330 mg, about 350 mg, about 400 mg, about 500 mg, about 550 mg, about 600 mg, about 625 mg, about 650 mg, about 700 mg, about 750 mg, about 900 mg, about 1000 mg, about 1050 mg, about 1200 mg, about 1250 mg, about 1600 mg or about 2000 mg of a cyclophilin-binding compound such as SCY-635, or a pharmaceutically acceptable salt, solvate or hydrate thereof. In a preferred embodiment, provided herein are unit dosage formulations that comprise about 200 mg, about 300 mg, about 350 mg, about 400 mg or about 500 mg of a cyclophilin-binding compound such as SCY-635, or a pharmaceutically acceptable salt, solvate or hydrate thereof.

In an embodiment, provided herein is the use of a cyclophilin-binding compound in the manufacture of a medicament for treating a patient having a disease susceptible to treatment with the cyclophilin-binding compound and a positive test for at least one cyclophilin-binding compound marker, said marker being a polymorphism residing in a region within about 5 kilobases (kb) of the IL28B gene, encoding interferon-lambda-3, said medicament comprising at least one cyclophilin-binding compound and at least one pharmaceutically acceptable carrier therefor. In a preferred embodiment, the disease is a viral disease, such as hepatitis C virus. In another embodiment, provided herein is a pharmaceutical composition for use in treating a patient having a disease susceptible to treatment with the cyclophilin-binding compound and a positive test for at least one cyclophilin-binding compound marker comprising a cyclophilin-binding compound, wherein the cyclophilin-binding compound marker is a polymorphism residing in a region within about 5 kilobases (kb) of the IL28B gene, encoding interferon-lambda-3, said composition comprising at least one cyclophilin-binding compound and at least one pharmaceutically acceptable carrier therefor. In a preferred embodiment, the disease is a viral disease, such as hepatitis C virus.

In another embodiment, provided herein are methods for maintaining a steady state average plasma concentration of a cyclophilin-binding compound such as SCY-635, or a pharmaceutically acceptable salt, solvate or hydrate thereof, of greater than about 250 ng/ml, about 275 ng/ml, about 300 ng/ml, about 350 ng/ml, about 475 ng/ml, or about 900 ng/ml, in a human subject for at least about 4, 6, 8, 12 or 24 hours or longer, comprising administering an effective amount of a cyclophilin-binding compound such as SCY-635, or a pharmaceutically acceptable salt, solvate or hydrate thereof, to a human subject infected with, or at risk for infection with, HCV. In certain embodiments, provided herein are methods for maintaining a steady state average plasma concentration of SCY-635, or a pharmaceutically acceptable salt, solvate or hydrate thereof, of greater than about 250 ng/ml in a human subject for at least about 4, 6, 8, 12 or 24 hours or longer, comprising administering an effective amount of a cyclophilin-binding compound such as SCY-635, or a pharmaceutically acceptable salt, solvate or hydrate thereof, to a human subject infected with, or at risk for infection with, HCV.

In various examples of the methods described herein, it is understood that depending on a genotype, a practitioner can select a treatment comprising a combination of any or all of a higher dose, longer duration, and/or more frequent administration when a genotype analysis as described herein indicates that a patient is less susceptible treatment with a cyclophilin-binding compound such as SCY-635, or can select a therapy not including a cyclophilin-binding compound such as SCY-635. Conversely, where a genotype analysis as described herein indicates susceptibility of a patient to treatment with a cyclophilin-binding compound such as SCY-635, a practitioner can choose a combination of any or all of a lower dose, less frequent administration, shorter duration, and the like. In some examples, the practitioner can choose to administer a cyclophilin-binding compound such as SCY-635 without one of more common co-therapies or as a single therapy.

A drug product can comprise a pharmaceutical composition and prescribing information, wherein the pharmaceutical composition comprises a cyclophilin-binding compound and the prescribing information comprises a pharmacogenetic indication, wherein the pharmacogenetic indication comprises the treatment of a disease susceptible to treatment with the cyclophilin-binding compound in patients who test positive for at least one cyclophilin-binding compound response marker, wherein the cyclophilin-binding compound response marker is a polymorphism in the patient residing about 3 kilobases (kb) upstream of the IL28B gene, encoding interferon-lambda-3. In one aspect of this embodiment the polymorphism is a C/T polymorphism, identified in the SNP rs 12979860 in the NCBI SNP Database allele of the IL28b gene.

A screening method for selecting individuals for initial treatment or continued treatment with a cyclophilin-binding compound from a group of individuals having a disease susceptible to treatment with a cyclophilin-binding compound can comprise testing each member of the disease group for the presence of at least one cyclophilin-binding compound response marker and selecting for treatment at least one individual testing positive for the cyclophilin-binding compound response marker, wherein a positive test for the cyclophilin-binding compound response marker is a heterozygous genotype or a homozygous genotype for the better response allele for a polymorphism in the patient residing about 3 kilobases (kb) upstream of the IL28B gene, encoding interferon-lambda-3.

A kit for testing an individual having a disease susceptible to treatment with a cyclophilin-binding compound for the presence or absence of a cyclophilin-binding compound response marker can comprise an oligonucleotide or a set of oligonucleotides designed to genotype at least one polymorphic site residing about 3 kilobases (kb) upstream of the IL28B gene, encoding interferon-lambda-3.

Cyclophilin-binding compounds such as SCY-635 have been discovered to stimulate OAS1. These compounds are restoring the OAS1 pathway for interferon-alpha, or a pathway indistinguishable from interferon-alpha. The OAS proteins have been shown to be important in attenuating infection in experimental respiratory syncitial virus and picornavirus cell culture infection systems. Failure of human immunodeficiency virus-1 (HIV-1) infected cells to release virus has been correlated with high concentrations of OAS and/or 2-5A. Furthermore, HIV-1 transactivator protein (tat) has been shown to block activation of OAS (Muller et al., J Biol. Chem. Mar. 5, 1990: 265(7):3803-8) thus indicating that novel forms of OAS might evade HIV-1 defence mechanisms and provide an effective therapy.

Thus, methods of treating a subject in need thereof wherein the subject has a condition, disease, or other indication that is treatable with IFN-α can comprise administering a cyclophilin-binding compound, for example SCY-635. In some examples, methods of treating a subject for a disease, condition, or other indication that is susceptible to treatment with IFN-α can comprise administration of SCY-635. In some examples, it is unnecessary to administer IFN-α. Such conditions include, but are not limited to, hepatitis C, hepatitis B, neoplastic disorders susceptible to IFN-α including malignant melanoma, use in patients with neurofibromatosis to shrink neurofibromas, hairy cell leukemia, AIDS-related Kaposi's sarcoma, chronic myelogenous leukemia, and genital and perianal warts caused by human papillomavirus (HPV).

The following examples illustrate the correlation of SCY-635 to genotype. These examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### Example 1

SCY-635 (given as oral capsules) was examined in a randomized, double-blind, placebo-controlled, multi-dose study to adult volunteers who are chronically infected with hepatitis C (subtype 1). Seven human subjects (1 placebo and 6 active) received 300 milligrams of SCY-635 three times per day (300 mg t.i.d.) per os for 15 consecutive days. Human subjects were excluded if they demonstrated evidence of co-infection with HIV-1, HBV, decompensated liver function, hepatocellular carcinoma, ALT values 2.5 times the upper limit of normal, or if they were the recipient of an organ transplant. All human subjects were male (n=20); 75% were African American; patients 0068-0073 were all African American. Average age was 53.0 years. Average HCV RNA plasma viremia on enrollment was 5,600,000 IU/ml, as measured by the quantitative Roche COBAS taqMan assay.

Blood and urine samples were collected from human subjects on Days 1, 2, 3, and 14 during the 8-hour interval immediately following administration of the first dose on each specified study day. In addition, blood samples for the measurement of trough drug concentrations were collected from all human subjects immediately prior to the administration of SCY-635 on the mornings of Days 4, 5, 8, 11, 12, and 15, and prior to the evening dose on Day 13.

The study showed that SCY-635 was well tolerated at all dose levels and no serious adverse events were reported during the study. Mild and moderate adverse events were reported; however, no evidence of a dose limiting toxicity was observed.

Viral load data from the study described was measured by the quantitative Roche COBAS taqMan assay. The test measures HCV RNA in International Units (IU) per mL using real-time Polymerase Chain Reaction (RT-PCR) technology. It quantitates HCV RNA from 10 to 100,000,000 IU/mL. All clinical viral load samples were assayed at LabCorp. The maximum log₁₀ change in HCV RNA was measured. Also the Day 3 plasma Cₘᵢₙ concentration of SCY-635 was noted.

In addition, blood samples from patients were taken and assayed to identify which of the three possible genotypes for the rs12979860 SNP was applicable. The tests were conducted following the procedure described in Ge et al. and were performed at LabCorp. The following results were obtained:

| | | Max Log₁₀ | | Day 3 |
|---|---|---|---|---|
| Patient | HCV | Change in | IL28B | Plasma Cₘᵢₙ |
| No. | Genotype | HCV RNA | Genotype | (ng/ml) |
| 0068 | 1b | 0.40 | CT | 0 |
| 0072 | 1a | 0.84 | TT | 396 |
| 0069 | 1b | 1.42 | CT | 403 |
| 0067 | 1a | 1.47 | CT | 244 |
| 0073 | 1a | 2.34 | CC | 232 |
| 0070 | 1a | 2.44 | CT | 607 |
| 0071 | 1a | 5.47 | CC | 518 |

Patient 68 received a placebo dose containing no SCY-635. All the other patients received SCY-635.

The results in the table above indicate a clear correlation between the patients' IL-28B genotype SNP and the degree of response to a cyclophilin inhibitor, in particular a cyclosporine derivative such as SCY-635, with the most profound response shown in the patients having the C/C SNP, a lower response demonstrated for the patients having the C/T SNP and the lowest response seen in patients with the T/T SNP.

### Example 2

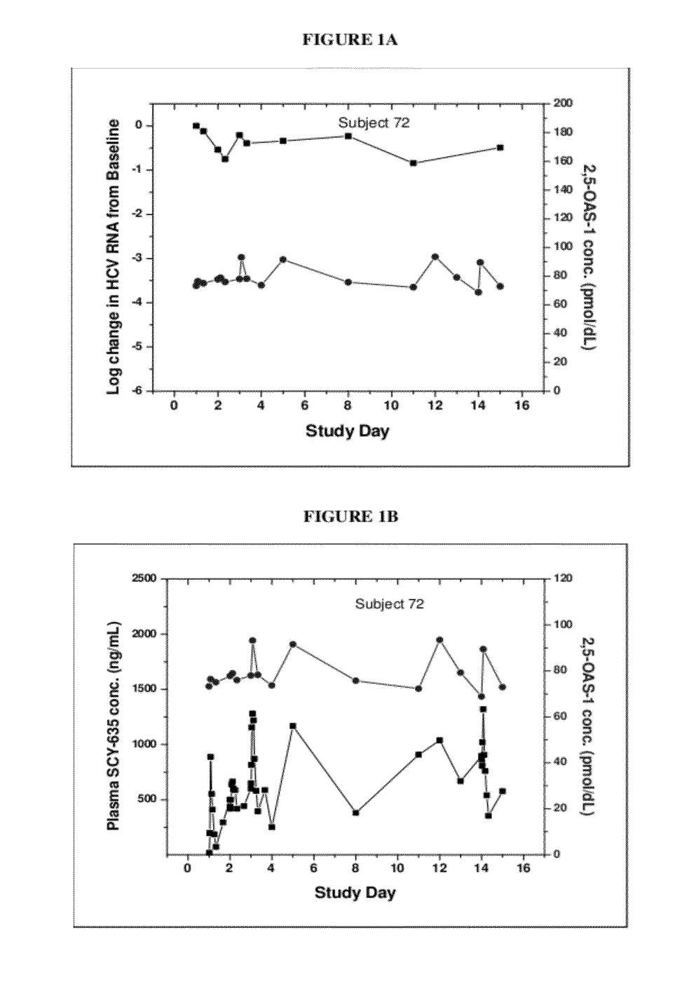

Plasma samples of the patient's blood in Example 1 above were also analyzed to measure the presence of human 2,5-oligoadentylate Synthetase 1 (OAS1). OAS1 is a member of the 2-5A synthetase family, essential proteins involved in the innate immune response to viral infection. The encoded protein is induced by interferons and uses adenosine triphosphate in 2′-specific nucleotidyl transfer reactions to synthesize 2′,5′-oligoadenylates (2-5As). These molecules activate latent RNase L, which results in viral RNA degradation and the inhibition of viral replication. The three known members of this gene family are located in a cluster on chromosome 12. Mutations in this gene have been associated with host susceptibility to viral infection. Alternatively spliced transcript variants encoding different isoforms have been described. OAS1 has been implicated as a major interferon stimulating gene activating antiviral RNAses (see for example Hoofnagle and Seeff, New England Journal of Medicine, Volume 355 (Dec. 7, 2006) pages 2444-2451, see in particular FIG. 1). Identification of OAS genes are described in U.S. Pat. No. 7,732,177 and OAS-like genes and mutations are described in U.S. Patent Publication numbers 2008/0081780, 2006/0275802 and 2005/0191649.

Blood plasma samples were taken from patients in Example 1 (see above) prior to dosing of SCY-635 and at time periods through the end of the fifteen days treatment and were tested using a Enzyme-linked Immunosorbent Assay (ELISA) kit for human OAS1 (obtained from Uscn Inc., the E92684Hu 96 Test). The kit is a sandwich enzyme immunoassay for the in vitro quantitative measurement of human OAS1 in serum, plasma and other biological fluids.

The microtiter plate provided in the kit has been pre-coated with a monoclonal antibody specific to OAS1. Standards or samples are then added to the appropriate microtiter plate wells with a biotin-conjugated polyclonal antibody preparation specific for OAS1. Next, Avidin conjugated to Horseradish Peroxidase (HRP) is added to each microplate well and incubated. A tetramethylbenzidine (TMB) substrate solution is added to each well. Only those wells that contain OAS1, biotin-conjugated antibody and enzyme-conjugated Avidin will exhibit a change in color. The enzyme-substrate reaction is terminated by the addition of a sulfuric acid solution and the color change is measured spectrophotometrically at a wavelength of 450 nm±10 nm. The concentration of OAS1 in the samples is then determined by comparing the optical density of the samples to the standard curve sample.

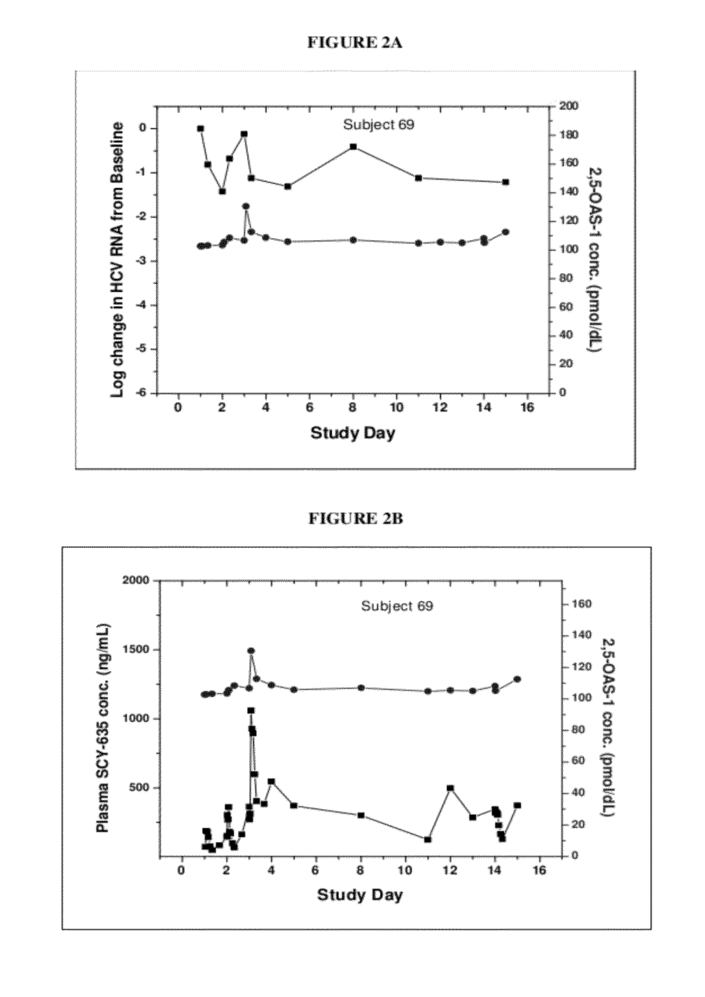

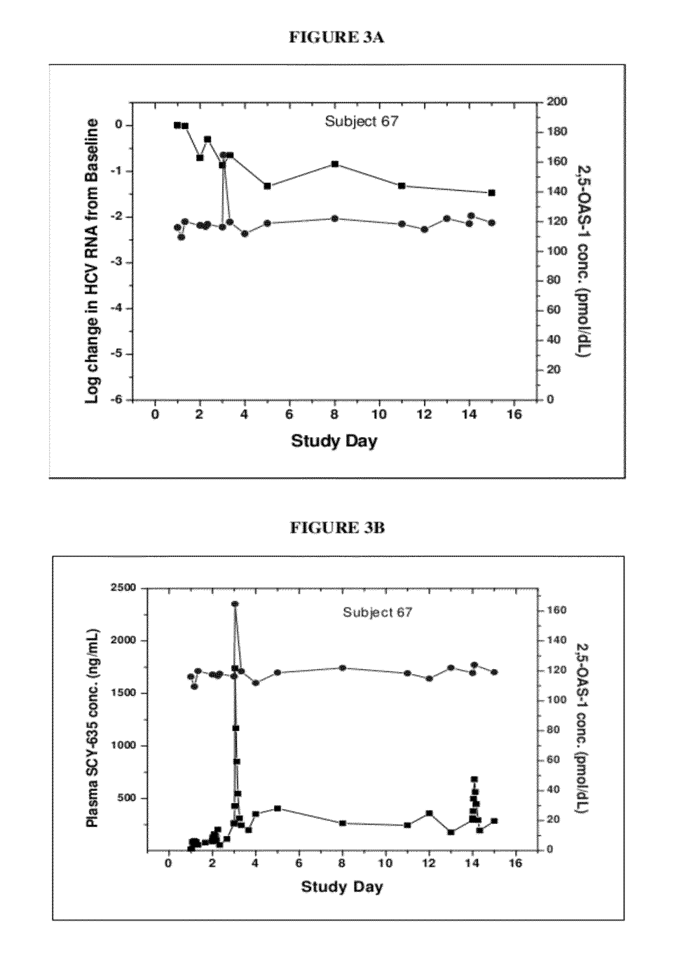

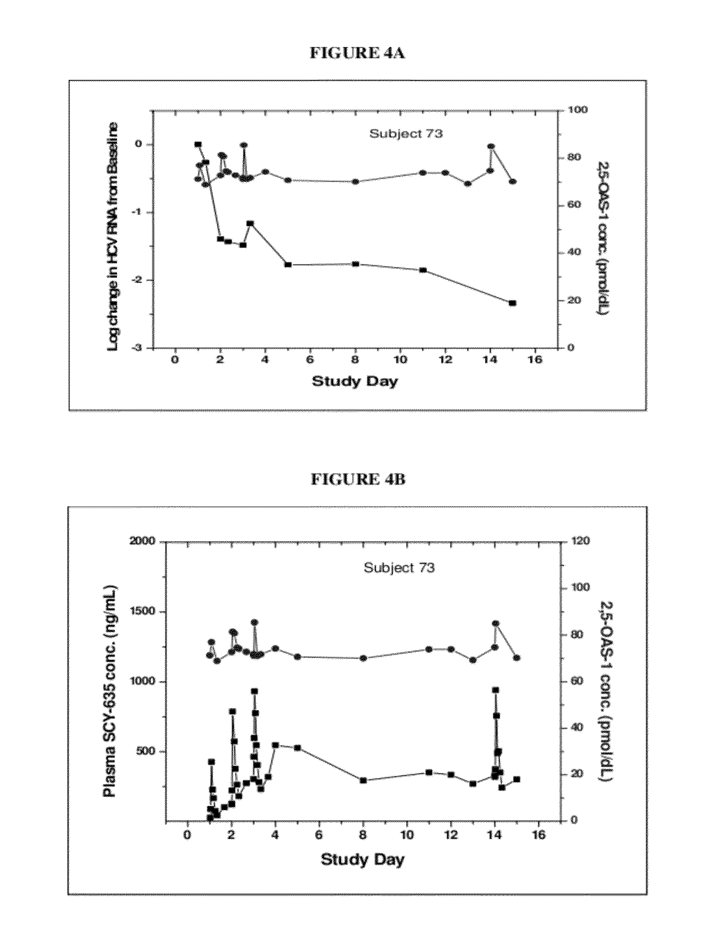

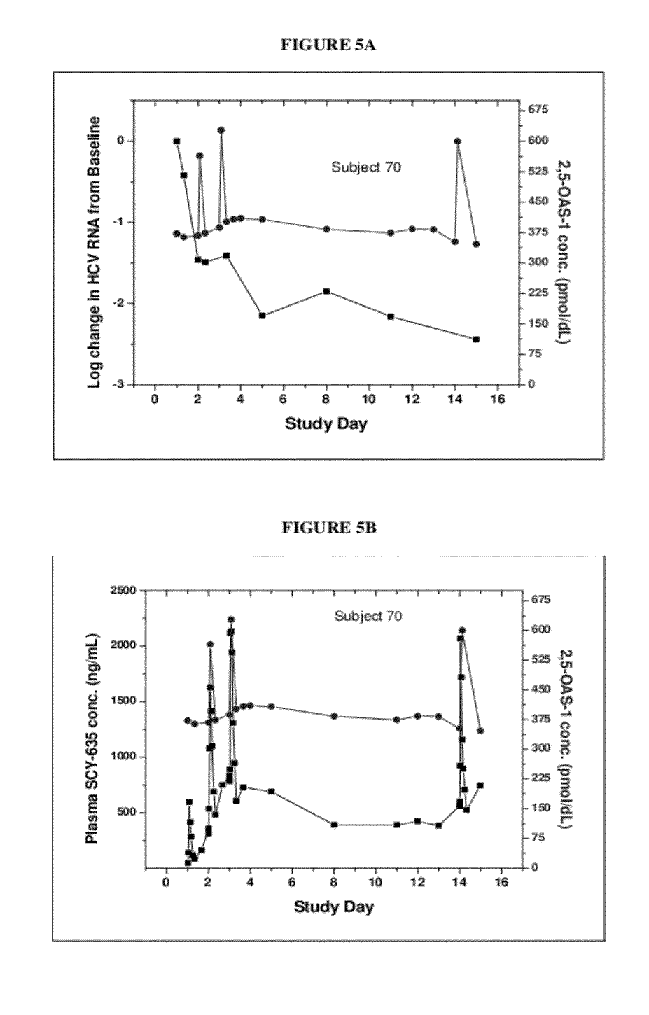

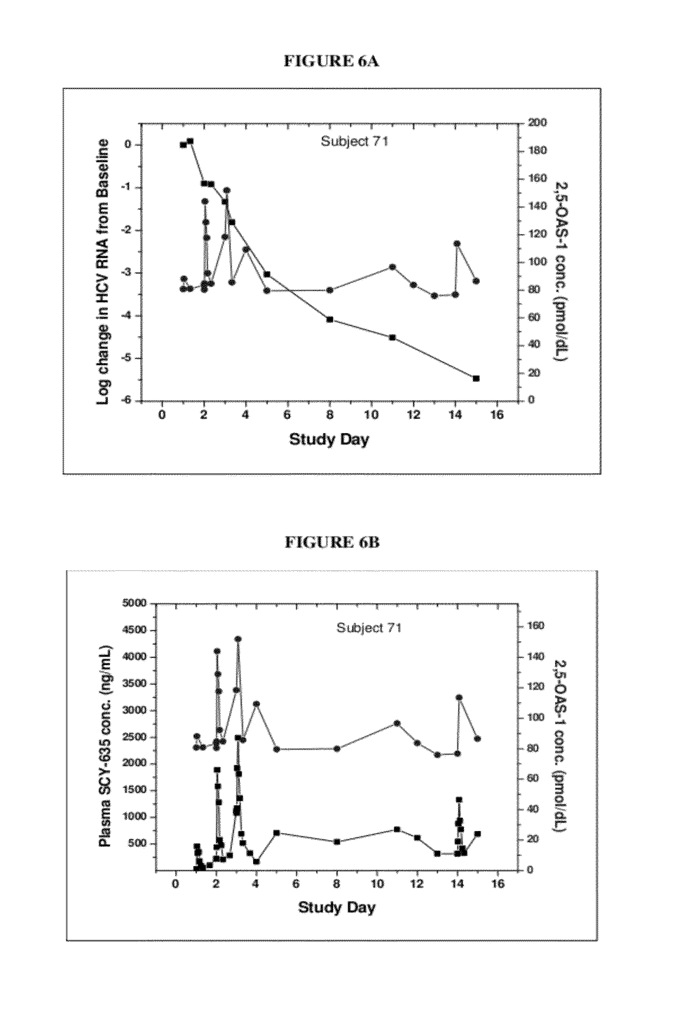

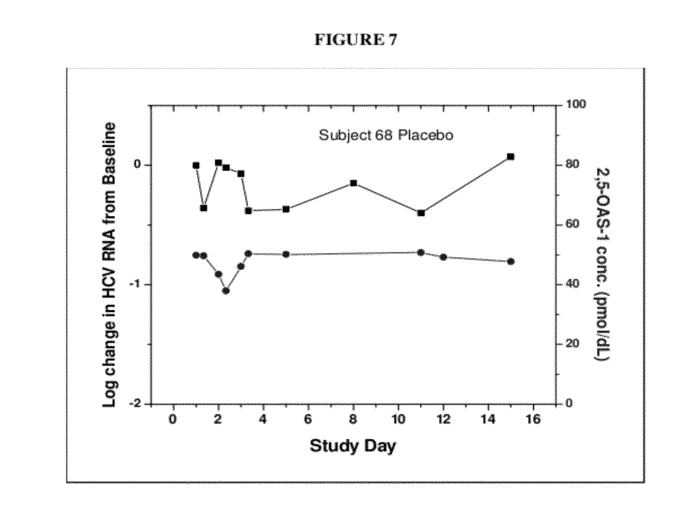

To calculate the results, the average for the duplicate readings for each standard, control, and sample (single reading) were taken and the average zero standard optical density was subtracted. A best fit straight line with OAS1 concentration on the y-axis and absorbance on the x-axis was drawn. Representative data are illustrated in FIGS. 1A-7.

The stimulation of OAS1 by cyclophilin-binding compounds such as SCY-635 indicates that these compounds are restoring the OAS1 pathway for interferon-alpha, or a pathway indistinguishable from interferon-alpha.

### Example 3

Plasma pharmacokinetic samples were obtained from all subjects who received a 900 milligram total daily dose of SCY-635 (given as 300 mg t.i.d.). The samples were analyzed by ELISA to determine their concentrations of interferon alpha (IFN-α), interferon beta (IFN-β), interferon lambda-1 (IFN-λ1/IL-29), and interferon lambda-3 (IFN-λ3/IL-28B).

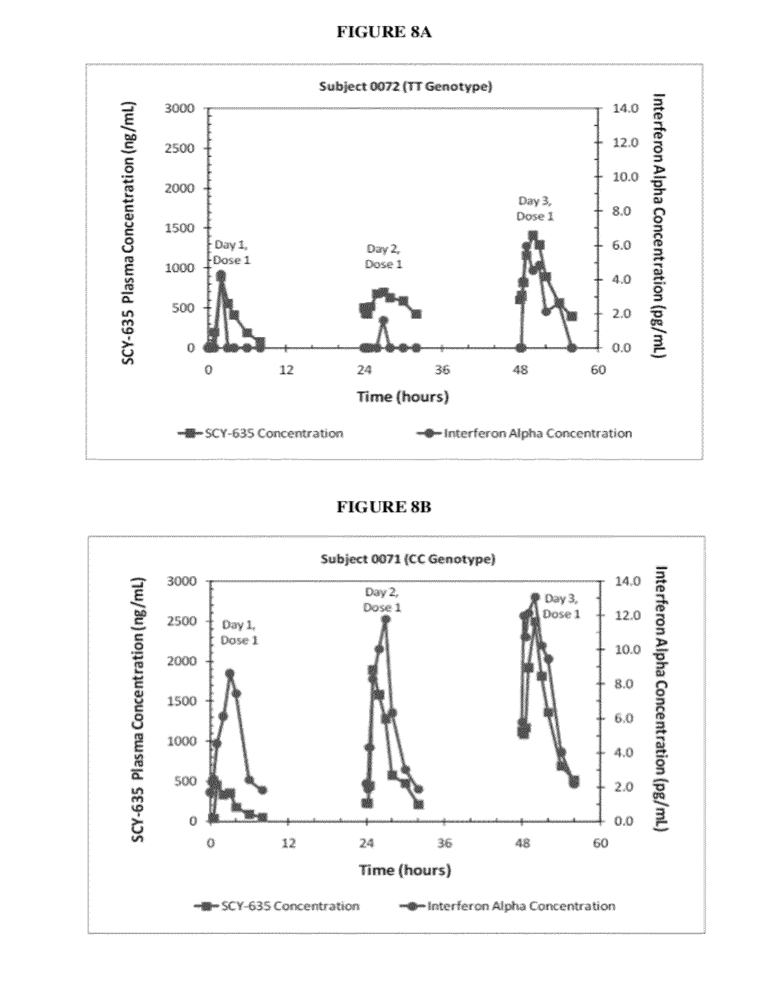

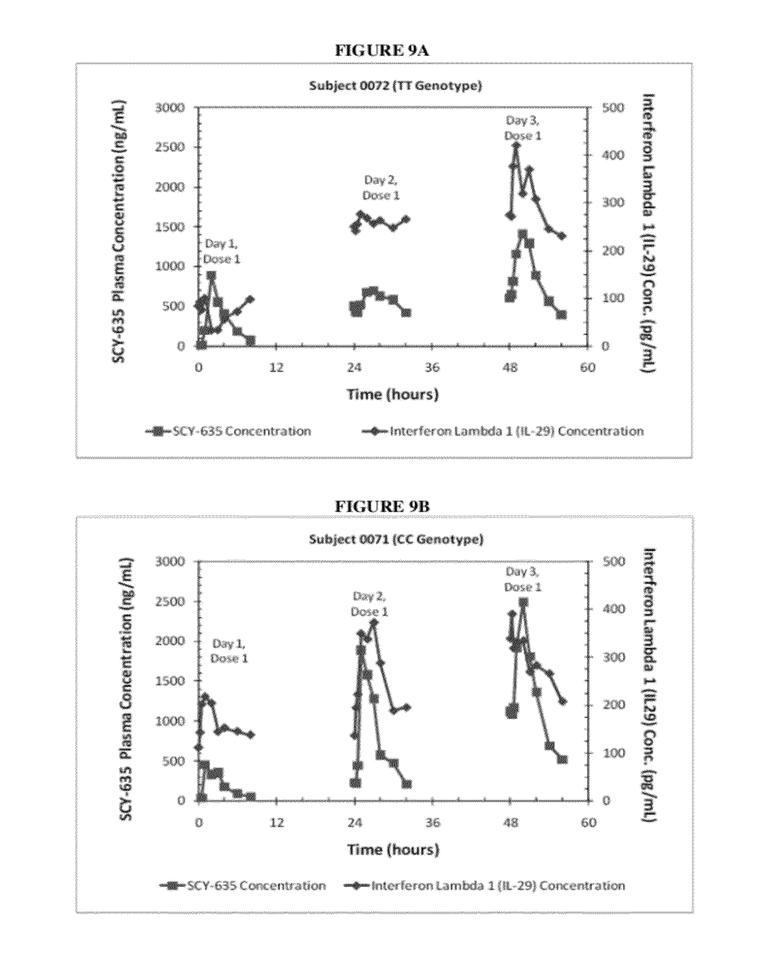

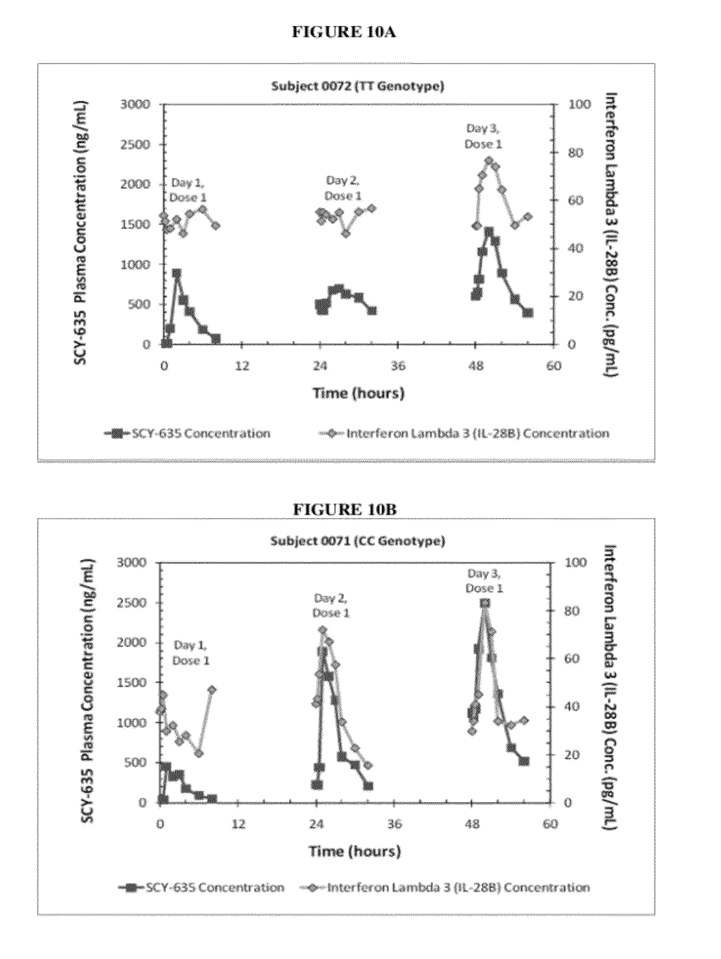

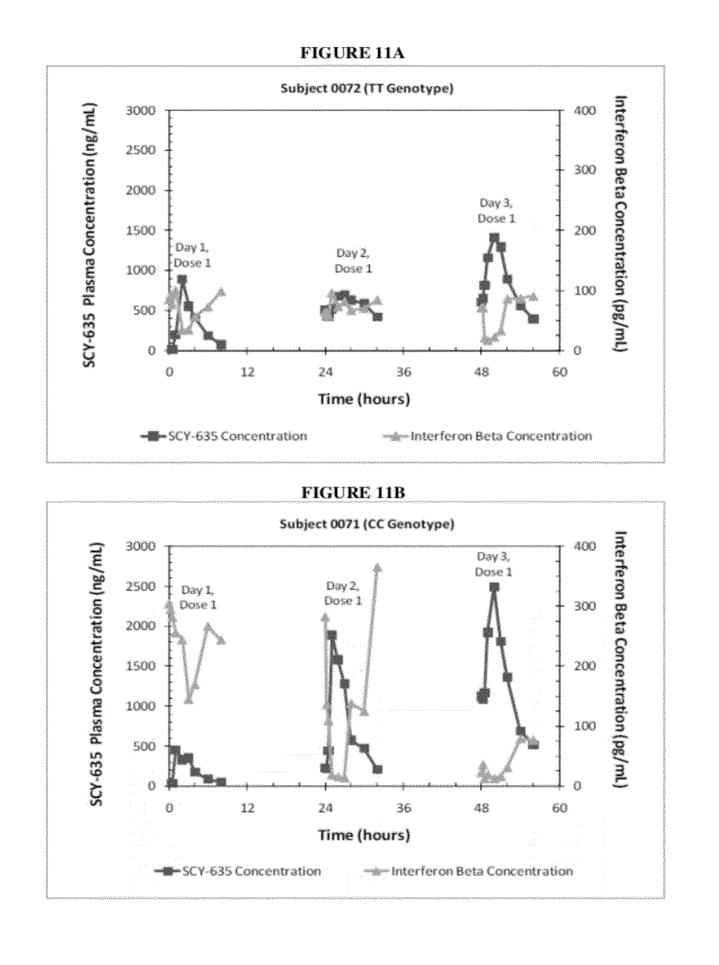

The following figures contain representative results obtained for Subject Number 0072 and Subject Number 0071 for IFN-α (FIGS. 8A and 8B), IFN-λ1/IL-29 (FIGS. 9A and 9B), IFN-λ3/IL-28B (FIGS. 10A and 10B), and IFN-β (FIGS. 11A and 11B). Subject Number 0072 exhibited the homozygous TT allele for IL28B. Subject Number 0071 exhibited the homozygous CC allele for the IL28B genotype.

Plasma samples for the determination of SCY-635 concentrations were taken throughout the 8-hour interval immediately following administration of the first dose of study medication on treatment days 1, 2, and 3. In FIGS. 8A-11B, the squares correspond to SCY-635 plasma concentrations following dose 1 on Study Day 1, dose 1 on Study Day 2, and dose 1 on Study Day 3. The left Y-axis contains the scale for the absolute plasma concentration of SCY-635. The diamond symbols correspond to the plasma concentrations for the various interferons. The opposing (right) Y-axis contains the scale for the absolute concentration of each respective interferon.

All subjects who received SCY-635 at a total daily dose of 900 milligrams exhibited increases from their respective baseline values for the plasma concentrations of IFN-α, IFN-λ1 and IFN-λ3. The highest concentrations of IFN-α, IFN-λ1, and IFN-λ3 were observed on Study Day 3, when the highest plasma concentrations of SCY-635 were achieved.

Changes in the plasma concentration of IFN-β were inversely related to changes in the plasma concentration of SCY-635 (i.e., an increase in SCY-635 concentration was accompanied by a coincident decrease in IFN-β concentration).

These data indicate that oral administration of SCY-635 to patients with chronic genotype 1 hepatitis C infection at a total daily dose of 900 milligrams regulates the expression of multiple species of endogenous interferons.

Treatment-associated increases in the plasma concentrations of IFN-α, IFN-λ1, and IFN-λ3 were observed in all subjects who received SCY-635. The observed increases in the concentrations of IFN-α, IFN-λ1, and IFN-λ3 were coincident with the changes in the plasma concentration of SCY-635, suggesting that the concentration of the unmodified parent drug drives the increased expression and plasma distribution of IFN-α, IFN-λ1, and IFN-λ3. Comparable values for the maximum observed concentrations of IFN-α, IFN-λ1, and IFN-λ3 (either expressed in terms of absolute plasma concentration or in terms of fold-change relative to baseline) were observed for all SCY-635 treated subjects irrespective of each individual's IL28B genotype. The maximum observed plasma concentrations for IFN-α are comparable to values achieved for pegylated interferon alpha 2b following the administration of exogenous PEG-Intron at the approved dose of 1.5 μg/kg (De Leede et al.—J. of Interferon and Cytokine Research, Vol. 28, 113-122, 2008). The maximum plasma concentrations observed for IFN-λ1 are comparable to values achieved following the exogenous administration of pegylated IFN-λ1 over the range of dose levels used in investigational studies (Muir et al.) and in individuals who spontaneously clear acute hepatitis C infection (Langhans et al.). These data also indicate that treatment with SCY-635 up-regulates the production of IFN-λ3 (another Type III interferon that exhibits potent anti-HCV activity in vitro.

These observations strongly suggest that the treatment-associated production of pharmacologically relevant plasma concentrations of IFN-α and IFN-λ1 mediates the clinical antiviral effects associated with the administration of SCY-635 at a total daily dose of 900 milligrams. Furthermore these data indicate that cyclophilin A plays a heretofore undiscovered and undocumented role in regulating the expression of type I and type III interferons.

Treatment-associated decreases in the plasma concentrations of INF-β were observed in all subjects who received SCY-635. The observed decreases in the concentrations of INF-β were coincident with increases in the plasma concentrations of SCY-635, suggesting that the concentration of the unmodified parent drug suppresses the expression of INF-β. These data are consistent with previously published mechanistic studies which indicate that expression of INF-β is regulated through a pathway that involves the cyclophilin B dependent phosphorylation of IRF-3 (Obata et al.). Phosphorylation of IRF-3 and expression of INF-β is not observed in cells where the expression of cyclophilin B has been silenced using siRNA techniques. This data are consistent with these observations and indicate that inhibition of cyclophilin B catalytic activity by SCY-635 specifically suppresses the expression of INF-β.

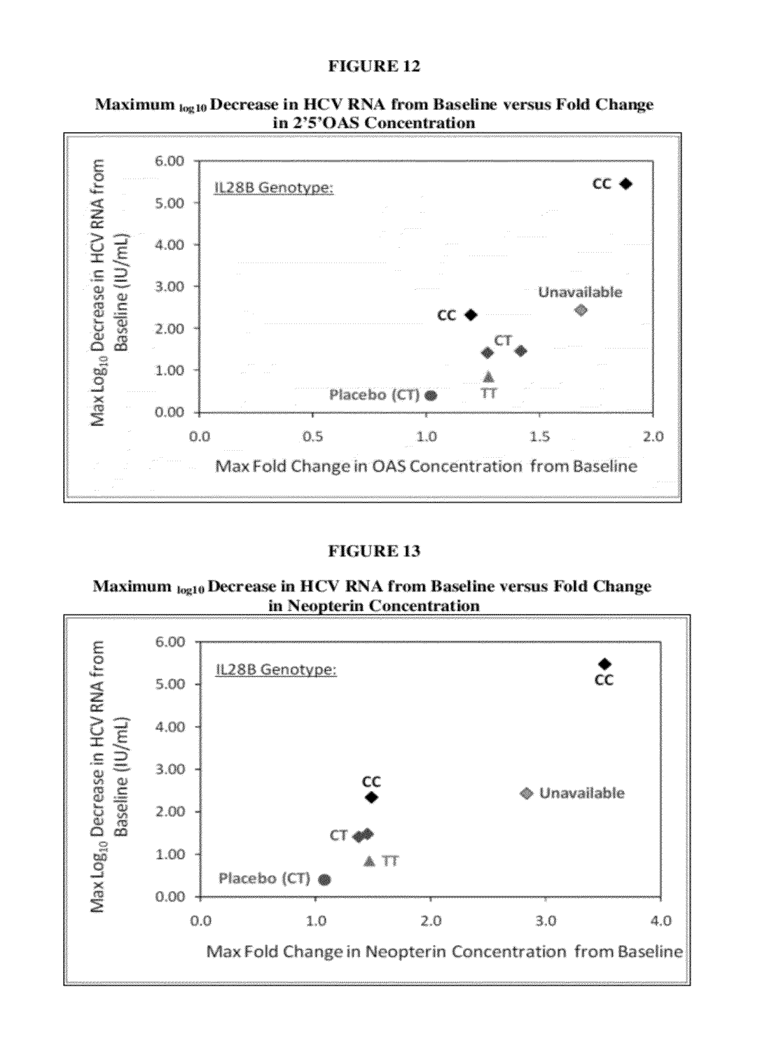

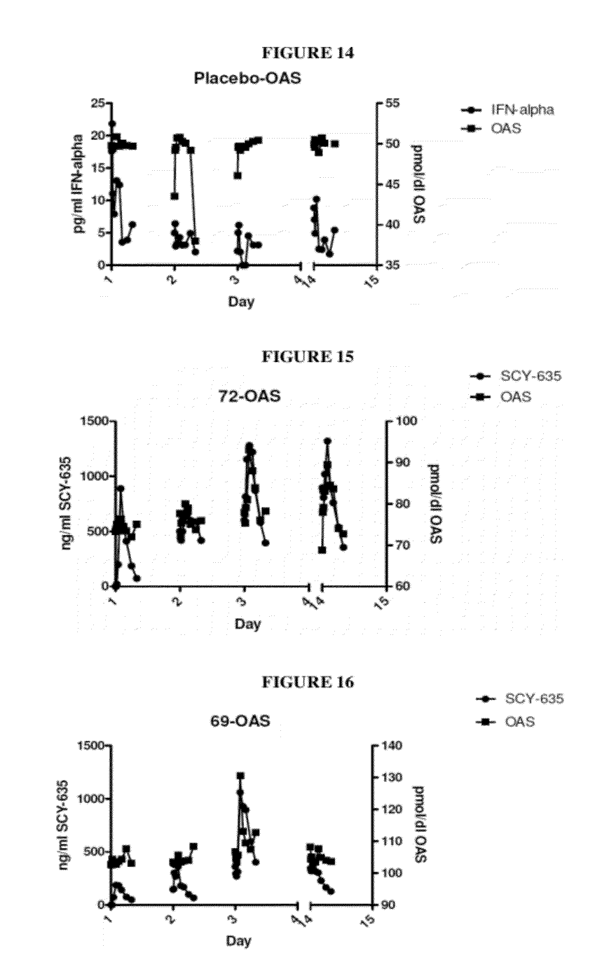

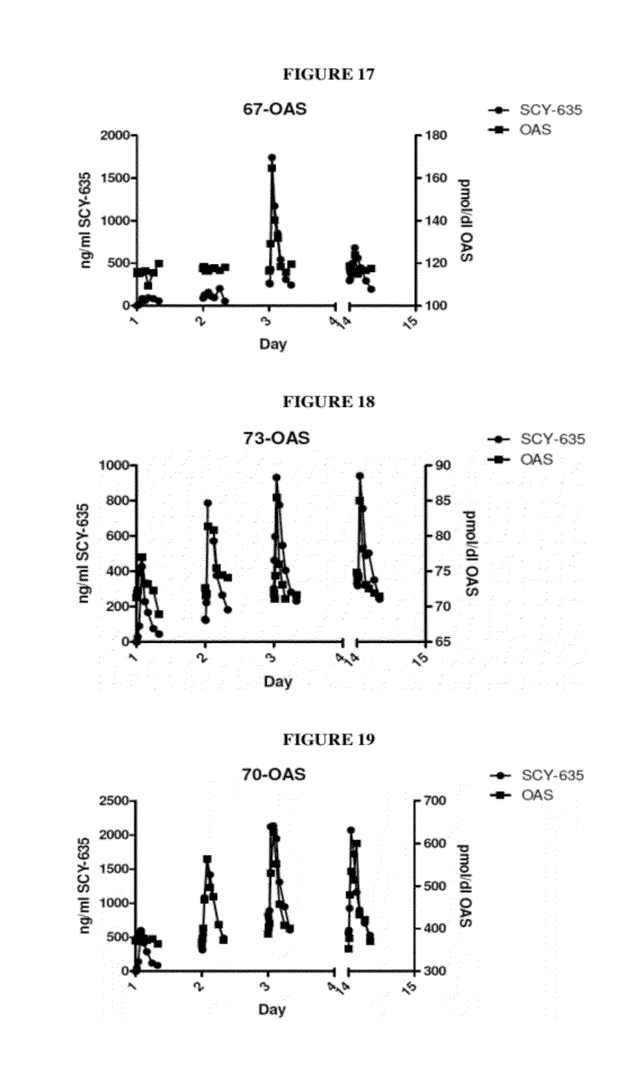

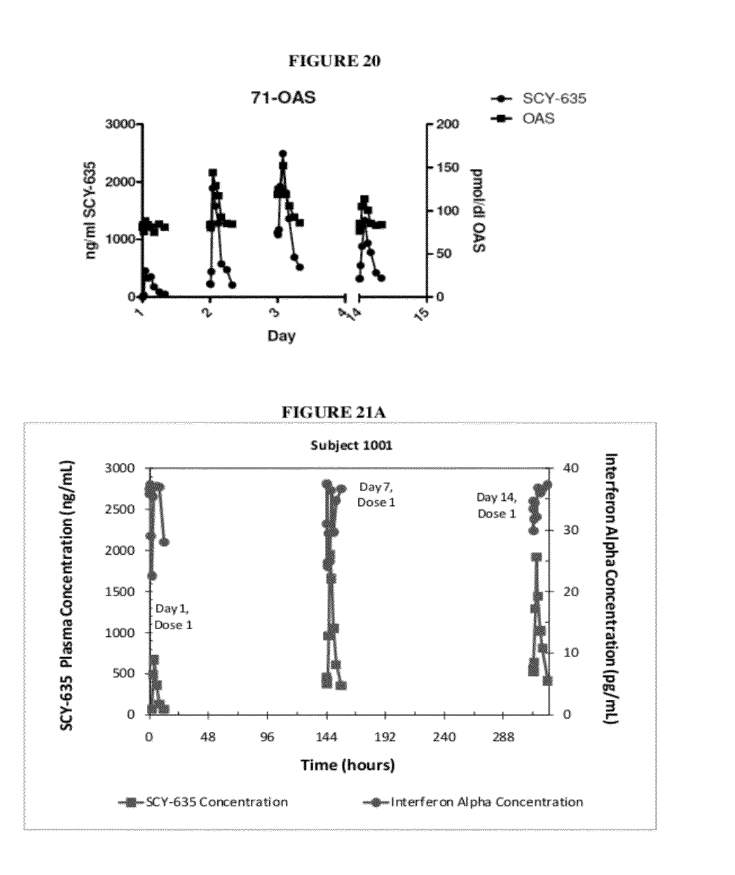

Patients who received the 900 milligram total daily dose of SCY-635 show varying degrees of innate immune activation that appears to correlate with response to treatment. The relationships between viral load nadir and maximum fold change in 2′S′ Oligoadenylate Synthetase; 2′5′OAS and neopterin, markers of immune system activation, is illustrated in FIGS. 12 and 13, respectively.

These results indicate that treatment with SCY-635 up-regulates the expression of multiple species of endogenous interferons, which in turn activates the JAK-STAT pathway and induces an antiviral state within the cell. The apparent relationship between the response to treatment with SCY-635 monotherapy and IL28B genotype is therefore explained by this set of observations, which demonstrates that exposure to SCY-635 results in the up-regulated expression of multiple endogenous interferons with potent antiviral activity. These observations are consistent with published reports by Ge et al., which demonstrate a relationship between IL28B genotype and response to interferon-based therapy. These observations further support the conclusion that the induction of pharmacologically relevant concentrations of type I and type III interferons represents the predominant mechanism through which SCY-635 exerts clinical anti-viral activity in patients with CHC.

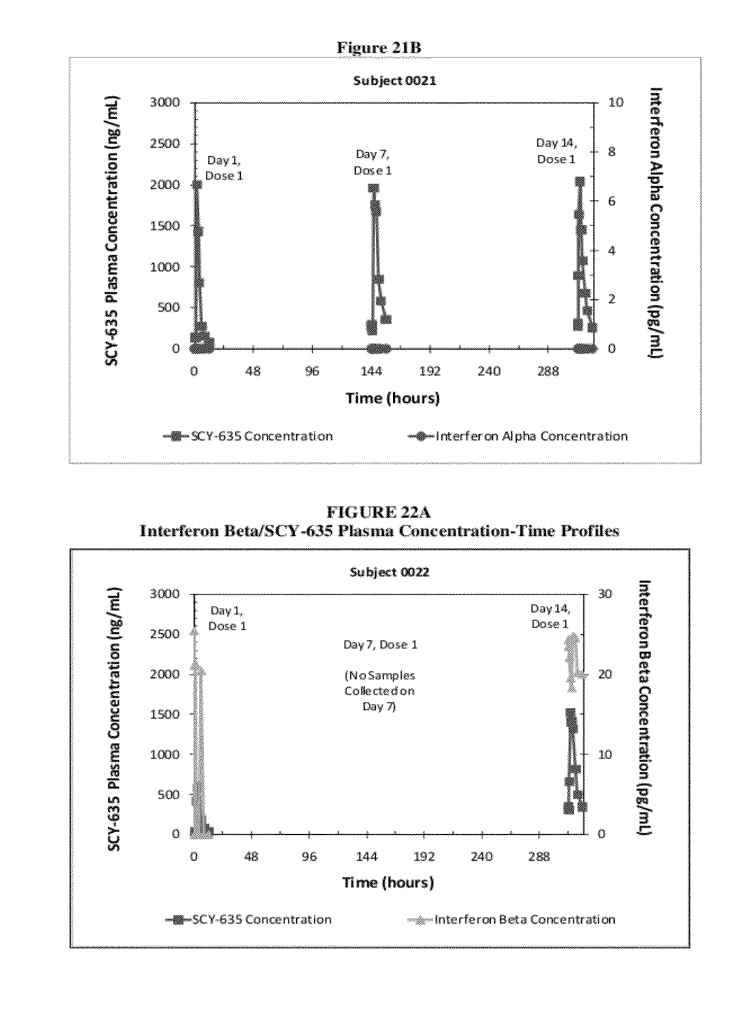

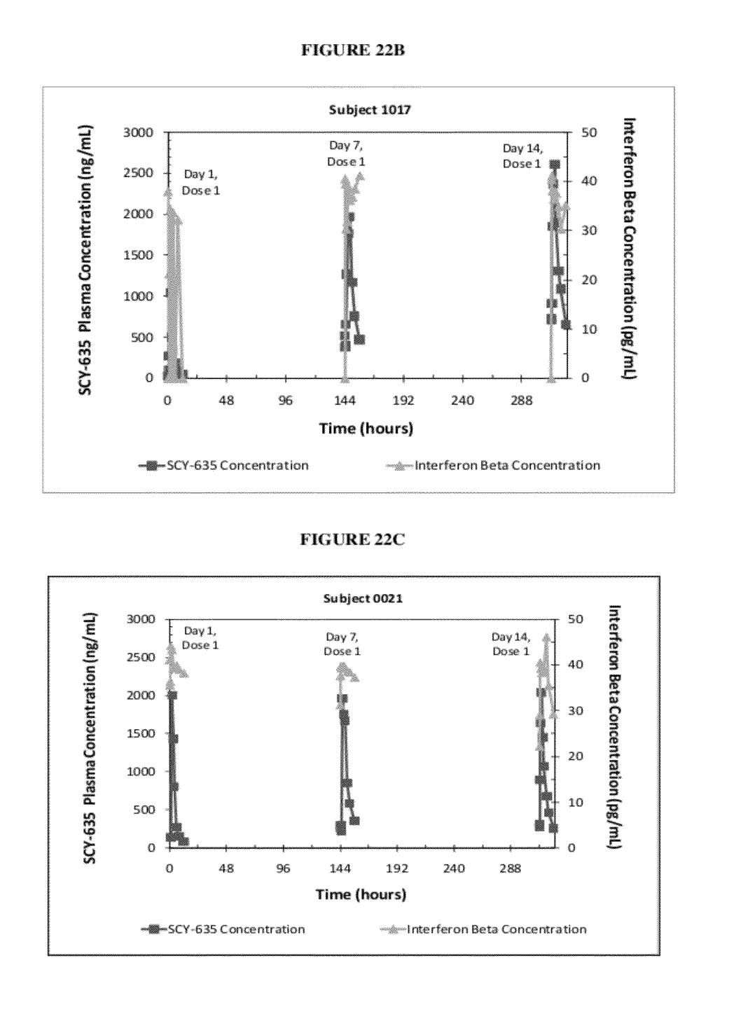

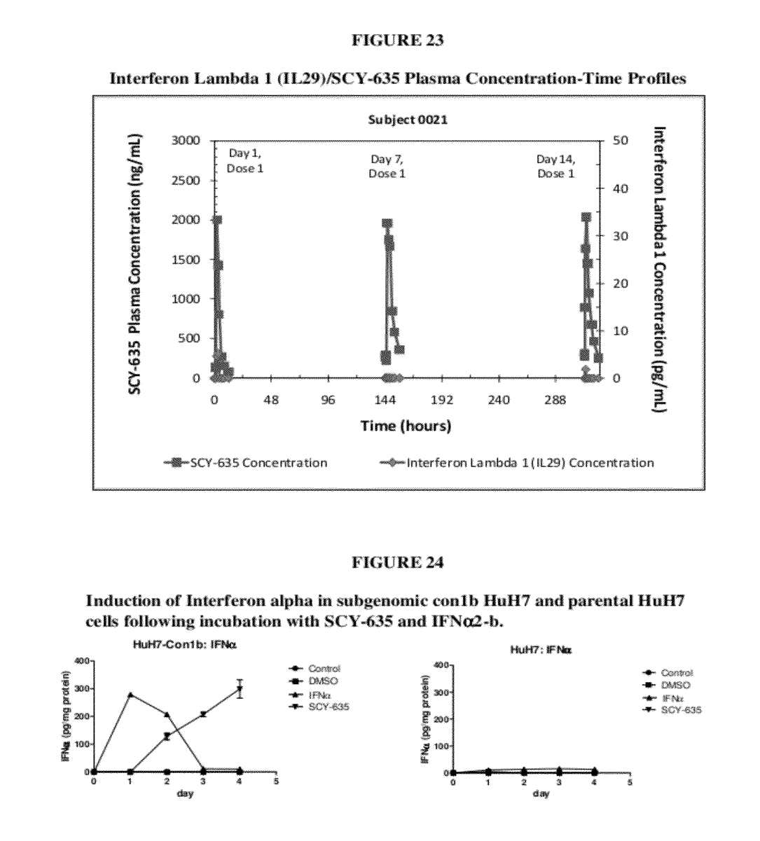

To establish whether the treatment-associated stimulation of endogenous interferon production is a virus dependent phenomenon, normal healthy volunteers who received SCY-635 at a total daily dose of 1,000 milligrams (500 mg b.i.d.) in a clinical study over 14 days can have samples taken and analyzed to determine their IL28B genotype and to assay for the presence of endogenous interferons. As illustrated in FIGS. 21A to 23, the dosing with SCY-635 did not result in a substantial change in the levels of interferon Alpha, Beta or Lambda 1.

Prospectively designed clinical studies can be performed with the purpose of establishing a dose and schedule for SCY-635 administration that could be considered as pharmacologically equivalent to the currently marketed forms of pegylated interferon alpha. These studies can enroll patients with CHC and evaluate the relationships between SCY-635 plasma concentration, regulation of interferon expression, innate immune activation, host IL28B genotype, and antiviral response. These studies can involve short duration monotherapy (i.e., a treatment period no greater than 5 days in total duration) in a relatively small number of patients stratified according to viral genotype and host IL28B genotype.

In an embodiment, SCY-635 can be used as an orally administered substitute for interferon alpha. In another embodiment, SCY-635 can be administered in combination with ribavirin in patients who are chronically infected with genotype 2 or 3 virus.

In a placebo-controlled, randomized, double-blind, dose-escalation study, patients with genotype 1 chronic HCV received placebo (n=3) or SCY-635 doses of 100 mg (n=6), 200 mg (n=5), or 300 mg (n=6) three times daily for 15 days. Safety, pharmacokinetics, and plasma HCV RNA were assessed and treatment-associated effects on innate immune function were evaluated.

SCY-635 administered at 300 mg/d or 600 mg/d was associated with minimal changes in HCV RNA. SCY-635 administered at 900 mg/d decreased HCV RNA in all dosed patients. On Day 15, the mean reduction in patients treated with SCY-635 at 900 mg/d was −2.24 log₁₀ IU/mL. Maximal decreases ranged from −0.84 to −5.47 log₁₀ IU/mL. All subjects who received SCY-635 at 900 mg/d exhibited treatment-dependent increases in the plasma protein concentrations of interferons α, λ₁ and λ₃ with temporally coincident increases in the plasma concentrations of 2′5′OAS1 and neopterin. Inter-individual variability in antiviral responses exhibited an apparent correlation with interferon expression, immune activation, and IL28B genotype with CC and CT subjects showing the greatest increases from baseline in endogenous interferons, 2′5′OAS1, and neopterin. No serious adverse events were reported, and no dose-limiting toxicities were observed.

Treatment with SCY-635 at 900 mg/d was associated with clinically relevant reductions of plasma HCV RNA in HCV patients. The apparent correlation between IL28B genotype and antiviral response suggests that the stimulation of pharmacologically relevant concentrations of endogenous interferons represents the primary mechanism through which SCY-635 exerts clinical antiviral activity.

Clinical studies demonstrated that 15 days of SCY-635 monotherapy at a total daily dose of 900 milligrams (administered as 300 milligrams t.i.d.) resulted in a mean maximal suppression of hepatitis C virus plasma RNA of −2.24 log 10 IU/mL below baseline. Individual maximum responses ranged from −0.84 to -5.4 log 10 IU/mL below baseline. Variation in plasma absorption of SCY-635 was low. After 3 days of treatment, when the highest plasma concentrations of SCY-635 were observed, the coefficients of variation (CV %) for plasma Cmin, AUCO-8, and Cmax were 37.0%, 41.5%, and 37.6% respectively. The 1L28B genotype of the patients was determined and the effects of treatment on innate immune function was assessed in order to understand factors contributing to the individual variation in antiviral response and to determine the mechanism of action for SCY-635 against chronic hepatitis C infection.

The plasma concentrations of neopterin and 2′5′OAS1 (markers of innate immune activation) and interferons α, β, λ1 (1L29), and λ3 (IL28B) were quantified from patient samples using commercially available ELISA-based assays. IL28B genotyping was performed using real time PCR with allele-specific Taqman probes to detect a single nucleotide polymorphism rs12979860 C/T on chromosome 18q13.

Subject number, IL28B genotype, and maximum antiviral response for 6 subjects who received 900 milligrams SCY-635/day were 72/TT/−0.84; 69/CT/−1.42; 67/CT/−1.47; 73/CC/−2.34; 70/CT/−2.44 and 71/CC/−5.47. All subjects who received active treatment exhibited SCY-635-dependent increases in the plasma protein concentrations of interferons α, λ1, and λ3 with concordant increases in the plasma protein concentration of 2′5′-OAS1 and neopterin. Interindividual variability in antiviral responses exhibited an apparent correlation with interferon expression, immune activation, and IL28B genotype. CC and CT patients exhibited greater increases from baseline for endogenous interferons, 2′5′-OAS1, and neopterin followed by TT patients. Placebo subjects showed no consistent changes in interferon expression, no immune activation, and no significant change in HCV-specific plasma RNA.

SCY-635 exerts clinical antiviral activity by upregulating the expression of multiple endogenous interferons. The apparent correlation between IL28B genotypes and the magnitude of antiviral responses to treatment with SCY-635 monotherapy suggests that the stimulation of pharmacologically relevant concentrations of endogenous interferons represents the primary mechanism through which SCY-635 exerts clinical anti-HCV activity.

### Example 4

### Determination of Interferon Levels in HCV-Infected or Uninfected Human Peripheral Blood Mononuclear Cells (PBMCs) when Treated with a Cyclophilin Inhibitor Compound

### Preparation of Primary Human PBMC Cells

Whole blood anti-coagulated with sodium citrate was obtained from healthy and HCV donors that had provided informed consent before donation (Bioreclamation, Westbury, N.Y.). Blood was collected into CPT tubes (BD Vacutainer CPT™ Tube, BD Biosciences Discovery Labware, Franklin Lakes, N.J.) via venipuncture and processed according to the manufacturer's instruction. Briefly, after collection and prior to centrifugation, tubes were gently inverted 8 to 10 times. Tubes were then centrifuged at 1700×g for 20 minutes at room temperature. Tubes were shipped on ice and after receipt they were carefully opened in a biological safety cabinet. The mononuclear layer was collected and transferred to a 50 mL conical tube. Five milliliters of PBS (P3813, Sigma, St Louis, Mo.) were added to wash cells and tube was centrifuged at 300×g for 15 minutes (Sorvall T1 Centrifuge, ThermoScientific, Rockford, Ill.). The supernatant was discarded without disturbing the cell pellet and the cell pellet was resuspended by gently tapping the tube. PBS was added again and the tube was centrifuged for 15 more minutes at 300×g. The final supernatant was discarded and the pellet was resuspended with 5 mL of RPMI 1640 (R7388, Sigma, St Louis, Mo.). The number of PBMCs in the suspension was then determined by counting an aliquot using a hemocytometer. The cell suspension was then adjusted to 1×10⁶ cells/mL with RPMI.

Whole blood tubes for IL28B genotyping were also drawn and the genotyping was performed by Gentris (Durham, N.C.) using real time PCR with allele-specific Taqman® probes to detect the single nucleotide polymorphism rs12979860 C/T on chromosome 18q13.

### Determination of Cytokine Production in Human PBMCs

PBMCs isolated from healthy (uninfected) and HCV infected donors were cultured at 37° C. in RPMI cell culture medium in flat bottom 48 well-plate (353230, Falcon, BD, Franklin Lakes, N.J.). Each well received 360 μL of cell suspension (1×10⁶ cells/mL). Treatments (40 μL) were added to each well (2 wells per condition), and included RPMI (control), RPMI with DMSO (0.005%), Cyclosporin A (CsA) or SCY-635 both at 20 μM in RPMI (for a final treatment of 2 μM CsA or SCY-635). Plates were incubated for 24 h at 37° C. in 5% CO₂ incubator. At the end of the incubation, the plates were centrifuged at 200×g for 5 minutes. The cell supernatants were then collected and assayed by ELISA for cytokines IFN-α's, IFN-β, IFN-λ1

(IL-29) and 2′,5′-OAS-1. The cell pellet was washed twice with cold PBS and 100 μL of water was added. Plates were then placed at −80° C. until protein analysis. For the protein analysis, plates were thawed, scraped and the protein content of the cell suspension was determined using the BCA Protein Assay Kit (23227, ThermoScientific, Rockford, Ill.).

IL28B genotyping results for two healthy donors indicated one CT and one TT rs12979860 genotype. Four HCV infected donors comprised one TT and three CT rs12979860 genotypes. Table 3 shows the demographics and IL28B genotyping of the 2 healthy and 4 HCV donors.

TABLE 3

| | Donors type | | | | IL28B genotype |
|---|---|---|---|---|---|
| Donor # | of infection | Age | Gender | Race | (rs12979860) |
| 1 | Healthy | 54 | Male | Hispanic/ | CT |
| | | | | Black | |
| 2 | Healthy | 47 | Male | Black | TT |
| 1 | HCV | 57 | Male | African | TT |
| | | | | American | |
| 2 | HCV | 57 | Male | African | CT |
| | | | | American | |
| 3 | HCV | 57 | Female | Caucasian | CT |
| 4 | HCV | 57 | Male | Caucasian | CT |

In PBMCs from healthy donors treated with 2 μM SCY-635, 2 μM CsA, or 2 μM Alisporivir the levels of IFNβ and 2′,5′-OAS-1 did not change relative to the DMSO control. Treatment with SCY-635 or Alisporivir did induce some IFNα production. There was also a small increase in IFNλ1 in the PBMCs from one healthy donor (genotype TT) following treatment with SCY-635. Results are shown in Tables 4 and 5.

TABLE 4
Healthy donor # 1, IL28B genotype CT. Cytokine concentrations in PBMC
supernatants treated with medium or drugs at 2 μM for 24 hours.

| | IFN αs | | IFN β | | IL-29 (IFN λ1) | | 2′,5′-OAS-1 | |
|---|---|---|---|---|---|---|---|---|
| | | pg/mg | | pg/mg | | pg/mg | | pmol/mg |
| Treament | pg/mL | protein | pg/mL | protein | pg/mL | protein | pmol/dL | protein |
| Control | <LOQ | | <LOQ | | <LOQ | | <LOQ | |
| | <LOQ | | <LOQ | | <LOQ | | <LOQ | |
| DMSO | <LOQ | | <LOQ | | <LOQ | | <LOQ | |
| | <LOQ | | <LOQ | | <LOQ | | <LOQ | |
| SCY-635 | <LOQ | | <LOQ | | <LOQ | | 2.42 | 19.8 |
| | <LOQ | | <LOQ | | <LOQ | | <LOQ | |
| CsA | <LOQ | | <LOQ | | <LOQ | | <LOQ | |
| | <LOQ | | <LOQ | | <LOQ | | <LOQ | |
| Alisporivir | <LOQ | | <LOQ | | <LOQ | | 3.17 | 29.2 |
| | <LOQ | | <LOQ | | <LOQ | | 4.81 | 44.4 |
| LOQ | 25 | | 12.5 | | 15.625 | | 2.34 | |

TABLE 5
Healthy donor # 2, IL28B genotype TT. Cytokine concentrations in PBMC
supernatants treated with medium or drugs at 2 μM for 24 hours.

| | IFN αs | | IFN β | | IL-29 (IFN λ1) | | 2′,5′-OAS-1 | |
|---|---|---|---|---|---|---|---|---|
| | | pg/mg | | pg/mg | | pg/mg | | pmol/mg |
| Treament | pg/mL | protein | pg/mL | protein | pg/mL | protein | pmol/dL | protein |
| Control | <LOQ | | <LOQ | | <LOQ | | <LOQ | |
| | <LOQ | | <LOQ | | <LOQ | | <LOQ | |
| DMSO | <LOQ | | <LOQ | | 22.1 | 95.7 | <LOQ | |
| | <LOQ | | <LOQ | | 19.7 | 77.7 | <LOQ | |
| SCY-635 | <LOQ | | <LOQ | | 40.2 | 160.1 | <LOQ | |
| | <LOQ | | <LOQ | | 42.8 | 830 | 3.5 | 14.1 |
| CsA | <LOQ | | 18.3 | 81.1 | <LOQ | | <LOQ | |
| | <LOQ | | <LOQ | | <LOQ | | <LOQ | |
| Alisporivir | <LOQ | | 16.1 | 56.7 | 19.2 | 67.7 | 2.5 | 8.7 |
| | <LOQ | | <LOQ | | 16.3 | 57.1 | 2.6 | 9.2 |
| LOQ | 25 | | 12.5 | | 15.625 | | 2.34 | |

Treatment of PBMCs from HCV infected donors with 2 μM SCY-635, 2 μM CsA, or 2 μM Alisporivir generally resulted in increased production of IFNα, OAS-1, and IFNλ1 compared to DMSO treatment. In contrast, the amount of IFNβ produced in the presence of drug was equal or less than the DMSO control. PBMCs from the three genotype CT donors have a higher production of IFNα, OAS-1, and IFNλ1 than the genotype TT donor. Results are shown in Tables 6-9.

TABLE 6
HCV donor # 1, IL28B genotype TT. Cytokine concentrations in PBMC
supernatants treated with medium or drugs at 2 μM for 24 hours.

| | IFN αs | | IFN β | | IL-29 (IFN λ1) | | 2′,5′-OAS-1 | |
|---|---|---|---|---|---|---|---|---|
| | | pg/mg | | pg/mg | | pg/mg | | pmol/mg |
| Treament | pg/mL | protein | pg/mL | protein | pg/mL | protein | pmol/dL | protein |
| Control | <LOQ | | <LOQ | | <LOQ | | <LOQ | |
| | <LOQ | | <LOQ | | <LOQ | | <LOQ | |
| DMSO | <LOQ | | <LOQ | | <LOQ | | <LOQ | |
| | <LOQ | | <LOQ | | <LOQ | | <LOQ | |
| SCY-635 | <LOQ | | <LOQ | | <LOQ | | <LOQ | |
| | <LOQ | | <LOQ | | <LOQ | | <LOQ | |
| CsA | <LOQ | | <LOQ | | <LOQ | | <LOQ | |
| | <LOQ | | <LOQ | | <LOQ | | <LOQ | |
| Alisporivir | <LOQ | | <LOQ | | <LOQ | | <LOQ | |
| | <LOQ | | <LOQ | | <LOQ | | <LOQ | |
| LOQ | 6.25 | | 100 | | 5 | | 9.38 | |

TABLE 7
HCV donor # 2, IL28B genotype CT. Cytokine concentrations in PBMC
supernatants treated with medium or drugs at 2 μM for 24 hours.

| | IFN αs | | IFN β | | IL-29 (IFN λ1) | | 2′,5′-OAS-1 | |
|---|---|---|---|---|---|---|---|---|
| | | pg/mg | | pg/mg | | pg/mg | | pmol/mg |
| Treament | pg/mL | protein | pg/mL | protein | pg/mL | protein | pmol/dL | protein |
| Control | <LOQ | | <LOQ | | <LOQ | | 14.00 | 159.8 |
| | <LOQ | | <LOQ | | <LOQ | | <LOQ | |
| DMSO | <LOQ | | <LOQ | | <LOQ | | 13.83 | 12939 |
| | <LOQ | | <LOQ | | <LOQ | | 10.00 | 77.3 |
| SCY-635 | <LOQ | | <LOQ | | 6.2 | 317.7 | 9.6 | 457.8 |
| | <LOQ | | <LOQ | | 7.6 | 530.4 | 9.7 | 388 |
| CsA | <LOQ | | <LOQ | | 6.7 | 403.4 | 10.4 | 611.8 |
| | <LOQ | | <LOQ | | 7 | 551.7 | <LOQ | |
| Alisporivir | <LOQ | | <LOQ | | <LOQ | | <LOQ | |
| | <LOQ | | <LOQ | | 5.9 | 187.5 | <LOQ | |
| LOQ | 6.25 | | 100 | | 5 | | 9.38 | |

TABLE 8
HCV donor # 3, IL28B genotype CT. Cytokine concentrations in PBMC
supernatants treated with medium or drugs at 2 μM for 24 hours.

| | IFN αs | | IFN β | | IL-29 (IFN λ1) | | 2′,5′-OAS-1 | |
|---|---|---|---|---|---|---|---|---|
| | | pg/mg | | pg/mg | | pg/mg | | pmol/mg |
| Treament | pg/mL | protein | pg/mL | protein | pg/mL | protein | pmol/dL | protein |
| Control | <LOQ | | <LOQ | | <LOQ | | <LOQ | |
| | <LOQ | | <LOQ | | <LOQ | | <LOQ | |
| DMSO | <LOQ | | <LOQ | | <LOQ | | <LOQ | |
| | <LOQ | | <LOQ | | <LOQ | | <LOQ | |
| SCY-635 | 7.9 | 489.2 | <LOQ | | 17.5 | 821 | 23.9 | 1138 |
| | <LOQ | | <LOQ | | 19.7 | 793.3 | 26.8 | 1072 |
| CsA | <LOQ | | <LOQ | | 17.8 | 612.2 | 28.6 | 1021 |
| | <LOQ | | <LOQ | | 15.8 | 826.8 | 23.5 | 1237 |
| Alisporivir | 9.1 | 319.2 | <LOQ | | 9.9 | 415.9 | 23.6 | 983 |
| | 7.9 | 249.5 | <LOQ | | 7.1 | 376.8 | 20.2 | 962 |
| LOQ | 6.25 | | 100 | | 5 | | 9.38 | |

TABLE 9
HCV donor # 4, IL28B genotype CT. Cytokine concentrations in PBMC
supernatants treated with medium or drugs at 2 μM for 24 hours.

| | IFN αs | | IFN β | | IL-29 (IFN λ1) | | 2′,5′-OAS-1 | |
|---|---|---|---|---|---|---|---|---|
| | | pg/mg | | pg/mg | | pg/mg | | pmol/mg |
| Treament | pg/mL | protein | pg/mL | protein | pg/mL | protein | pmol/dL | protein |
| Control | <LOQ | | <LOQ | | <LOQ | | <LOQ | |
| | <LOQ | | <LOQ | | <LOQ | | <LOQ | |
| DMSO | <LOQ | | <LOQ | | <LOQ | | <LOQ | |
| | <LOQ | | <LOQ | | <LOQ | | <LOQ | |
| SCY-635 | 21 | 334.6 | <LOQ | | 19.7 | 313.2 | 34.8 | 552 |
| | 19.2 | 331.3 | <LOQ | | 19.7 | 344.5 | 31.6 | 554 |
| CsA | 9.3 | 277.1 | <LOQ | | 11.8 | 451.8 | 19.9 | 603 |
| | 11.2 | 328.4 | <LOQ | | 14.4 | 411.1 | 21.2 | 623 |
| Alisporivir | 13.7 | 174 | <LOQ | | 14.9 | 189 | 14.9 | 189 |
| | 11.3 | 141.1 | <LOQ | | 13.9 | 172.9 | 21.2 | 265 |
| LOQ | 6.25 | | 100 | | 5 | | 9.38 | |

These results suggest that the increased production of IFNα, OAS-1, and IFNλ1 seen in PBMCs following treatment with SCY-635, CsA, or Alisporivir requires HCV infection.

In a separate experiment, treatment of PBMCs from a HCV-infected donor with 2 μM SCY-635, 2 μM CsA, 2 μM Alisporivir, or 2 μM NIM-811 resulted in increased production of IFNα and IFN λ1 compared to DMSO treatment. Results are shown in Table 10 below.

TABLE 10
HCV donor # 2, IL28B genotype CT. Separate experiment from that
shown in Table 8. Cytokine concentrations in PBMC supernatants treated
with medium or drugs at 2 μM for 24 hours. Cellular ATP content,
measured using CellTiter-Glo Luminescent Assay (Promega, Madison,
Wisconsin) according to manufacturer's instructions, was used as
indicator of relative cell densities. In addition to SCY-635, cyclosporine
A, alisporivir and NIM-811, the following further representative
cyclosporine A derivatives were tested
(in the Table below ‘Cpd’ means Compound).

| | | | 5-Val | |
|---|---|---|---|---|
| Cpd | 3-Sar substituent | 4-position | substituent | 8-position |
| A | H | Gamma —OH | H | (D)-Ala |
| | | MeLeu | | |
| B | —CH₃ | MeLeu | —CH₂Ph | (D)-Ala |
| C | —SCH₂CH₂OCH₃ | MeLeu | H | (D)-Ala |
| D | —SCH₃ | Gamma —OH | H | (D)-Ala |
| | | MeLeu | | |
| E | —OCH₂CH₃ | Gamma —OH | H | (D)-Ala |
| | | MeLeu | | |
| F | —OCH₂CH₂OCH₃ | Gamma —OH | H | (D)-Ala |
| | | MeLeu | | |
| G | —SCH₂-(N- | Gamma —OH | H | (D)-Ala |
| | methylpyrazol-4-yl) | MeLeu | | |
| H | H | MeLeu | H | [(N,N-ε- |
| | | | | dimethyl)-D- |
| | | | | lysyl |
| I | H | MeLeu | —CH₂Ph | (D)-Ala |
| J | —SCH₂CH₂CH₃ | MeLeu | —CH₂Ph | (D)-Ala |
| K | —CH₃ | MeLeu | trans-3- | (D)-Ala |
| | | | methylbut-2- | |
| | | | enyl | |

The compounds are referred to as Compounds A to K below.

| | | | IL-29 (IFN λ1) | |
|---|---|---|---|---|
| | IFN αs | | | pg/uM |
| Treatment | pg/mL | pg/uM cellular ATP | pg/mL | cellular ATP |
| DMSO | <LOQ | | <LOQ | |
| | <LOQ | | <LOQ | |
| | <LOQ | | <LOQ | |
| SCY-635 | 13.2 | 8.2 | 23.5 | 14.6 |
| | 15.4 | 10.0 | 23.9 | 15.5 |
| | 14.0 | 8.9 | 23.9 | 15.1 |
| CsA | 7.6 | 5.7 | 10.2 | 7.7 |
| | 8.4 | 5.9 | 10.9 | 7.6 |
| Alisporivir | 19.5 | 12.2 | 19.9 | 12.5 |
| | 22.7 | 13.7 | 20.0 | 12.1 |
| NIM-811 | 14.5 | 10.0 | 15.8 | 10.9 |
| | 12.3 | 8.4 | 11.6 | 7.9 |
| Compound A | 20.2 | 7.2 | 20.5 | 7.3 |
| | 14.1 | 8.3 | 16.5 | 9.6 |
| Compound B | 21.1 | 10.4 | 22.8 | 11.2 |
| | 23.0 | 11.3 | 22.7 | 11.2 |
| Compound C | 17.8 | 8.6 | 15.7 | 7.6 |
| | 16.1 | 10.0 | 19.1 | 11.9 |
| Compound D | 13.1 | 7.9 | 29.8 | 18.0 |
| | 12.1 | 10.8 | 24.9 | 22.1 |
| Compound E | 14.8 | 7.9 | 13.8 | 7.4 |
| | 12.2 | 6.9 | 13.9 | 7.9 |
| Compound F | 17.6 | 9.8 | 12.6 | 7.0 |
| | 17.0 | 5.9 | 17.8 | 6.2 |
| Compound G | 14.4 | 8.0 | 20.6 | 11.4 |
| | 11.3 | 6.1 | 18.7 | 10.2 |
| Compound H | 13.3 | 5.9 | 15.3 | 6.8 |
| | 14.0 | 8.6 | 14.6 | 9.0 |
| Compound I | 6.0 | 7.7 | 5.1 | 6.5 |
| | 5.0 | 6.9 | 5.3 | 7.4 |
| Compound J | 4.3 | 2.5 | <LOQ | |
| | 3.0 | 1.6 | 6.3 | 3.4 |
| Compound K | 16.9 | 9.5 | 18.3 | 10.3 |
| | 16.4 | 9.5 | 21.6 | 12.5 |
| LOQ | 1.56 | | 3.9 | |

In one embodiment PBMCs isolated from a patient prior to beginning treatment with a cyclophilin-binding compounds are exposed in vitro to the cyclophilin-binding compound to evaluate the responding changes in production of interferons and the products of interferon-stimulated genes (e.g., OAS1). The results can confirm the susceptibility of the patient to treatment with the cyclophilin-binding compound, and may be further used in conjunction with genotype information to guide the selection of a treatment regimen.

### Example 5

### Effect of SCY-635 on Interferon and Interferon Stimulated Gene Induction in Subgenomic, con1b HuH7 Cells

Subgenomic con1b HuH7 and parent HuH7 cells were maintained in a humidified incubator at 37° C. in 5% CO2. Cells were cultured in Dulbecco's modified essential media (DMEM) supplemented with 10% fetal bovine serum (FBS), 1% penicillin-streptomycin, 1% glutamine, and 1% non-essential amino acids. Media for subgenomic con1b HuH7 cells also contained 5 mg/ml G418.

Subgenomic replicon cells were plated into 6-well dishes at a starting density of 100,000 cells per well in the presence of G418. Parental HuH7 cells were plated in parallel at a starting density of 50,000 cells per well to obtain similar densities to replicon cells on days 3 and 4. Five plates of each cell line were prepared; plates were incubated overnight in a humidified incubator at 37° C. in 5% CO2.

### Day 0

One plate of each cell line was harvested as follows;
Centrifuged the 6-well plate at approximately 700×g for 10 min at 4° C. in swinging bucket rotor.
Removed the extracellular medium from each well and placed in a 5 ml cryovial. Sealed the tube and placed on ice prior to freezing at −20° C.
Rinsed the cell monolayer with cold (4° C.) PBS by adding enough volume to cover the cell monolayer. Allowed the PBS to sit on the cell monolayer for approximately 15 seconds prior to removal with pipette.
Removed the PBS and discarded. Washed 2 more times.
Added 300 μL of distilled water to each well and sealed the plate.
Immediately put the plate and cryovials at −20° C.
   The remaining four plates were treated as follows in the absence of G418 selection;
One well received no treatment.
One well received DMSO (equivalent to amount added with drug)
Two wells received SCY-635 (final concentration 2 uM)
   Two wells received IFNα-2b (final concentration 50 U/ml)

### Day 1-4

Harvested one plate of each cell line each day as described above.

### Sample Preparation

Six-well plates were thawed and 1 mM PMSF was added to each well. Cell lysates were transferred to a deep 96-well plate and frozen at −20° C. Cryovials were thawed and 2.5 ml cell supernatants were transferred to a deep 96-well plate. Daughter plates with 130 ul supernatant per well were sealed and frozen at −20° C.

### Determination of Cytokine Production

IFNα in cell supernatants was measured using the Human IFNα Multi-Subtype ELISA kit (PBL 41105) following the kit protocol with two exceptions. The IFNα standard was diluted in complete DMEM media, and the high sensitivity standard curve was extended to include a 6.25 pg/ml value. IFNβ in cell supernatants was measured using the Human IFNβ ELISA kit (PBL 41410) following the kit protocol.

IFNλ1, which is the Interleukin 29 (IL29) gene product, and 2′-5′ Oligoadenylate Synthetase 1 (2′-5′ OAS) in cell supernatants were measured using the ELISA kit for IL29 or 2′-5′ OAS (USCN Life Sciences E92029Hu, E92684Hu) following the kit protocols. Combined IFNα levels were measured using the VeriPlex™ Human Interferon Multiplex ELISA kit (9-plex), following the kit protocols.

### Results

Incubation of subgenomic con1b HuH7 cells with 2 μM SCY-635 resulted in a steady increase in levels of IFNα production from day 1 to day 4. Treatment with 50 U/ml IFNα-2b caused endogenous IFNα production (i.e., elevation of detected IFNα to levels above those of the added IFNα-2b) that peaked at day 1 and declined by day 3. Incubation of parental HuH7 cells with SCY-635 or IFNα-2b did not result in changes in endogenous IFNα levels. The low levels of IFNα seen in HuH7 cells treated with IFNα-2b indicate detection of the exogenous drug. There were no changes in IFNα levels in untreated cells or cells incubated with DMSO. Results are shown in FIG. 24.

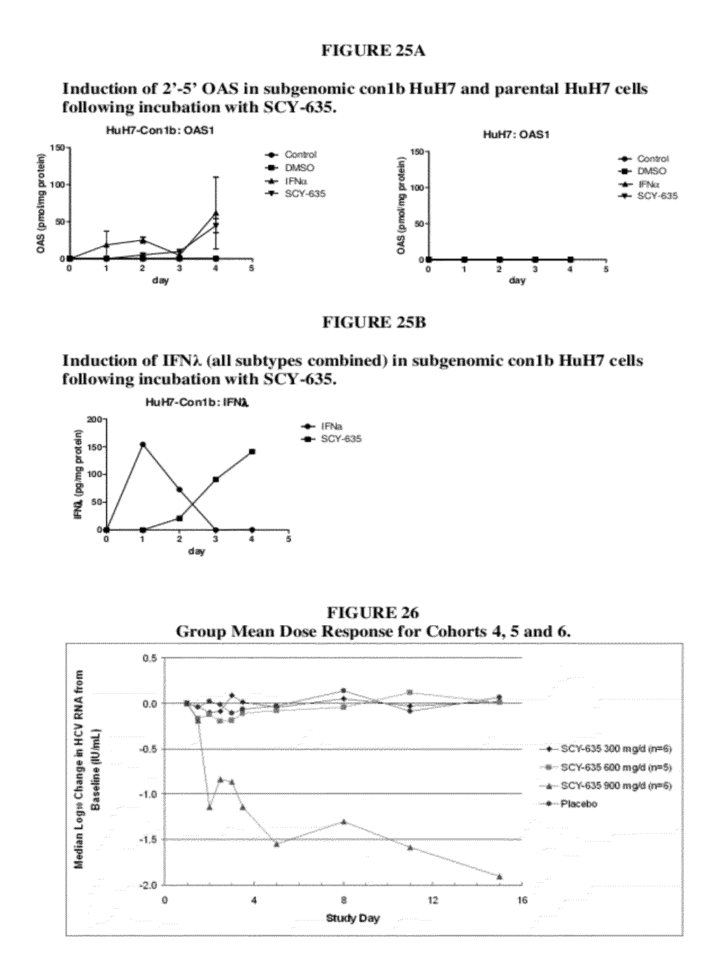

Incubation of subgenomic con1b HuH7 cells with 2 μM SCY-635 or 50 U/ml IFNα-2b also resulted in increased levels 2′-5′ OAS, which is an interferon stimulated gene (ISG). Untreated or DMSO treated replicon cells did not have any increase in 2′-5′ OAS. No 2′-5′ OAS was detected in supernatants from treated or untreated HuH7 cells. Results are shown in FIG. 25A.

Cell supernatants were assessed for IFNβ, IFNλ1, and total IFNα, production. IFNβ levels for all supernatants were below the limit of detection, which was 25 pg/ml. Levels of IFNλ1 detected in subgenomic con1b HuH7 cells treated with 2 μM SCY-635 or 50 U/ml IFNα-2b were close to or below the limit of detection at 31.2 pg/ml. The minimal amount of IFNλ1 production did not change over time, and it was also seen in the untreated or DMSO treated replicon cells as well as the HuH7 cells indicating that it represented background levels (data not shown). Changes in the combined IFNα levels (all subtypes detected) released by subgenomic con1b HuH7 cells treated with 2 μM SCY-635 or 50 U/ml IFNα-2b mirrored the changes observed in IFNα release (as shown in FIG. 25B).

These results suggest that the increased production of IFNα and OAS-1 seen in HuH7 cells following treatment with SCY-635 requires HCV infection.

### Example 6

A randomized, placebo-controlled, double-blind clinical study of SCY-635 given as monotherapy is conducted for 5 days to adult subjects chronically infected with HCV genotypes 1 and 4. Potential subjects are either treatment naïve or experienced subjects with evidence of relapse to prior treatment with PegIFNα and ribavirin. The study includes 3 treatment cohorts. Eligible subjects with HCV genotypes 1 or 4 are randomly assigned to receive treatment with either SCY-635 or matching placebo. The randomization is stratified on the basis of host IL28B genotype such that an equal number of C/C and non-C/C(C/T and T/T) subjects are represented in each cohort. The subjects who are randomized to receive placebo include patients with the C/C genotype and the non-C/C genotype.

Three nominal doses of SCY-635 are evaluated. The total daily doses of SCY-635 will be 600 milligrams (300 mg b.i.d.), 750 milligrams (250 mg t.i.d.), and 900 milligrams (300 mg t.i.d.). Subjects arrive at the clinic on the evening of Day-1 (Check-in) and remain in the clinic until Day 6. Subjects are discharged on the morning of Day 6 after all scheduled study assessments are performed. Subjects return to the clinical research unit for a follow-up visit on Day 13 (±2 days).

An intensive series of samples for pharmacokinetic and pharmacodynamic analyses are collected from all subjects on Days 1, 3, and 5 prior to the morning dose and following each daily dose (i.e., morning, afternoon and evening doses for the t.i.d. cohorts and morning and evening doses for the b.i.d. cohort). Samples for HCV—specific RNA (viral load) are obtained at all visits. Safety is monitored by evaluation of clinical adverse events, physical examinations, vital signs, electrocardiography, and clinical safety laboratory tests.

### Discussion of Further Results

Prior clinical studies demonstrated that 15 days of SCY-635 monotherapy at a total daily dose of 900 milligrams (administered as 300 milligrams t.i.d.) resulted in a mean maximal suppression of hepatitis C virus plasma RNA of −2.24 log 10 IU/mL below baseline. Individual maximum responses ranged from −0.84 to −5.4 log 10 IU/mL below baseline. Variation in plasma absorption of SCY-635 was low. After 3 days of treatment, when the highest plasma concentrations of SCY-635 were observed, the coefficients of variation (CV %) for plasma Cmin, AUC0-8, and Cmax were 37.0%, 41.5% and 37.6% respectively. We determined the IL28B genotype and assessed the effects of treatment on innate immune function in order to understand factors contributing to the individual variation in antiviral response and to determine the mechanism of action of SCY-635 against chronic hepatitis C infection.

The plasma concentrations of neopterin and 2′5′OAS-1 (markers of innate immune activation) and interferons α, β, λ1 (IL29) and λ3 (IL28) were quantified from patient samples using commercially available ELISA-based assays. IL28B genotyping was performed using real time PCR with allele-specific Taqman probes to detect a single nucleotide polymorphism rs12979860 C/T on chromosome 18q13. Informed consent was obtained from all subjects who participated in the study. Additional information on the subjects in cohorts 4-6 are provided in Table 11.

TABLE 11
Comparison of Host IL28B Genotype, HCV Genotype, Viral Load Decline and SCY-635 Plasma
Exposure for Cohorts 4 (100 mg t.i.d.), 5 (200 mg t.i.d.), and 6 (300 mg t.i.d.)

| | | | | | Max Log 10 | | |
|---|---|---|---|---|---|---|---|
| | Subject | | IL28B | HCV | Decline | Day 3 C8 hr | Day 3 AUC0-8 |
| Cohort | Number | Race | Genotype | Genotype | in HCV RNA | (ng/mL) | (hr*ng/mL) |
| 4 | 0057 | African | TT | 1b | 0.00* | 26.3 | 482 |
| | | American | | | | | |
| 4 | 0056 | African | TT | 1b | 0.12 | 29.1 | 338 |
| | | American | | | | | |
| 4 | 0053 | Caucasian | CT | 1b | 0.19 | 0** | 0** |
| | | American | | | | | |
| 4 | 0055 | African | CT | 1a | 0.22 | 27.6 | 245 |
| | | American | | | | | |
| 4 | 0058 | African | CT | 1a | 0.31 | 50.0 | 410 |
| | | American | | | | | |
| 4 | 0059 | African | CC | 1a | 0.46 | 38.5 | 644 |
| | | American | | | | | |
| 4 | 0054 | Other | CC | 1a | 0.52 | 26.4 | 282 |
| 5 | 0063 | African | CT | 1a | 0.11 | 0** | 0** |
| | | American | | | | | |
| 5 | 0060 | African | — | 1a | 0.17 | 38.1 | 1008 |
| | | American | | | | | |
| 5 | 0064 | Other | TT | 1a | 0.40 | 76.3 | 1455 |
| 5 | 0062 | Caucasian | CT | 1a | 0.42 | 93.0 | 1177 |
| | | American | | | | | |
| 5 | 0061 | African | TT | 1a | 0.50 | 96.4 | 1269 |
| | | American | | | | | |
| 5 | 0065 | Caucasian | CC | 1a | 0.89 | 127 | 2400 |
| | | American | | | | | |
| 6 | 0068 | African | CT | 1b | 0.40 | 0** | 0** |
| | | American | | | | | |
| 6 | 0072 | African | TT | 1a | 0.84 | 396 | 7090 |
| | | American | | | | | |
| 6 | 0069 | African | CT | 1b | 1.42 | 403 | 5353 |
| | | American | | | | | |
| 6 | 0067 | African | CT | 1a | 1.47 | 244 | 5307 |
| | | American | | | | | |
| 6 | 0073 | African | CC | 1a | 2.34 | 232 | 3776 |
| | | American | | | | | |
| 6 | 0070 | African | CT | 1a | 2.44 | 607 | 10960 |
| | | American | | | | | |
| 6 | 0071 | African | CC | 1a | 5.47 | 518 | 10590 |
| | | American | | | | | |

*Subject showed a 0.06 Log10 increase in viral load from baseline
**Subject received placebo
Other = Native Hawaiian/Other Pacific Islander

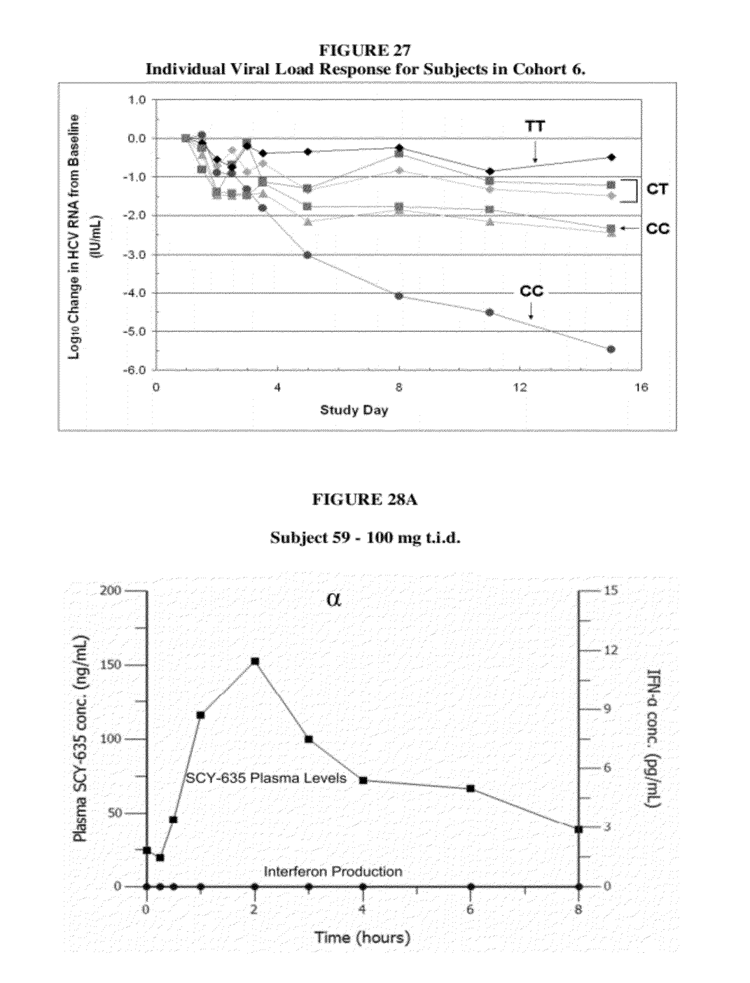

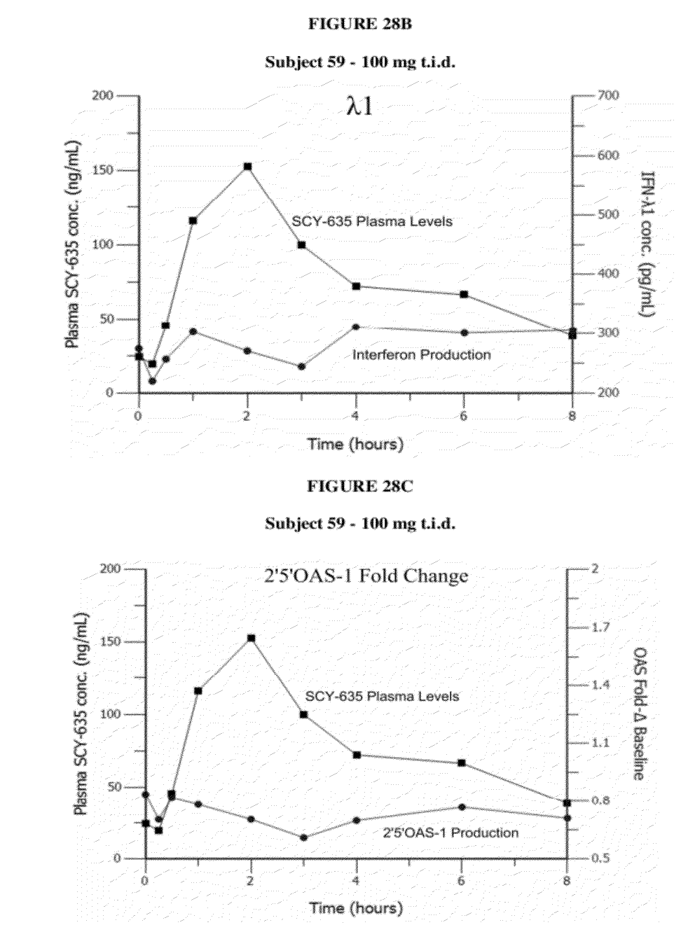

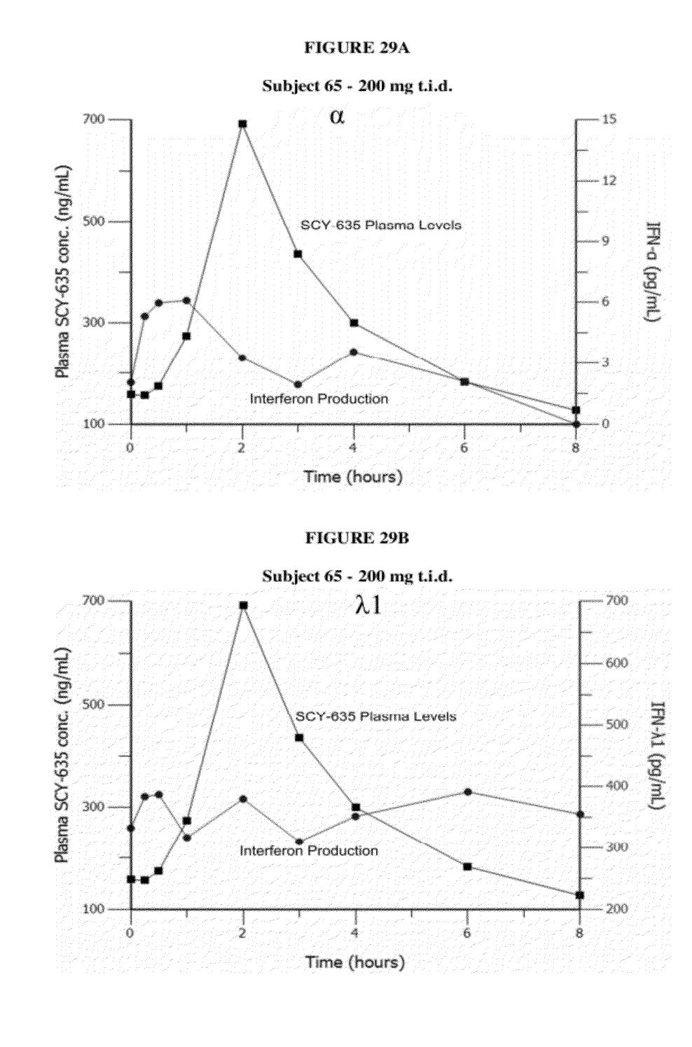

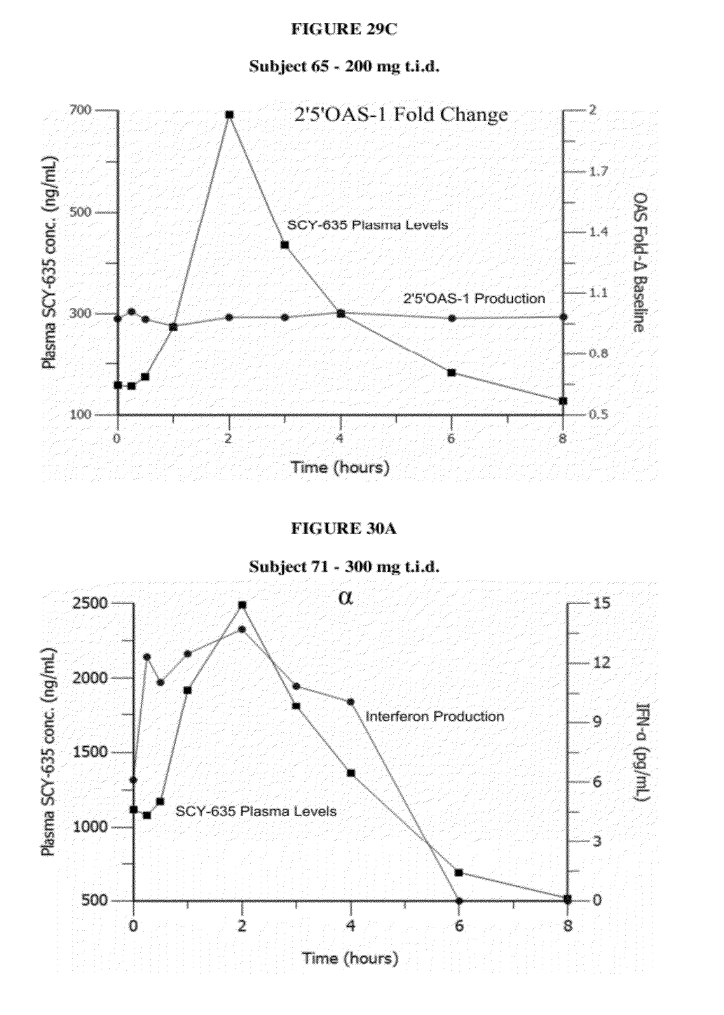

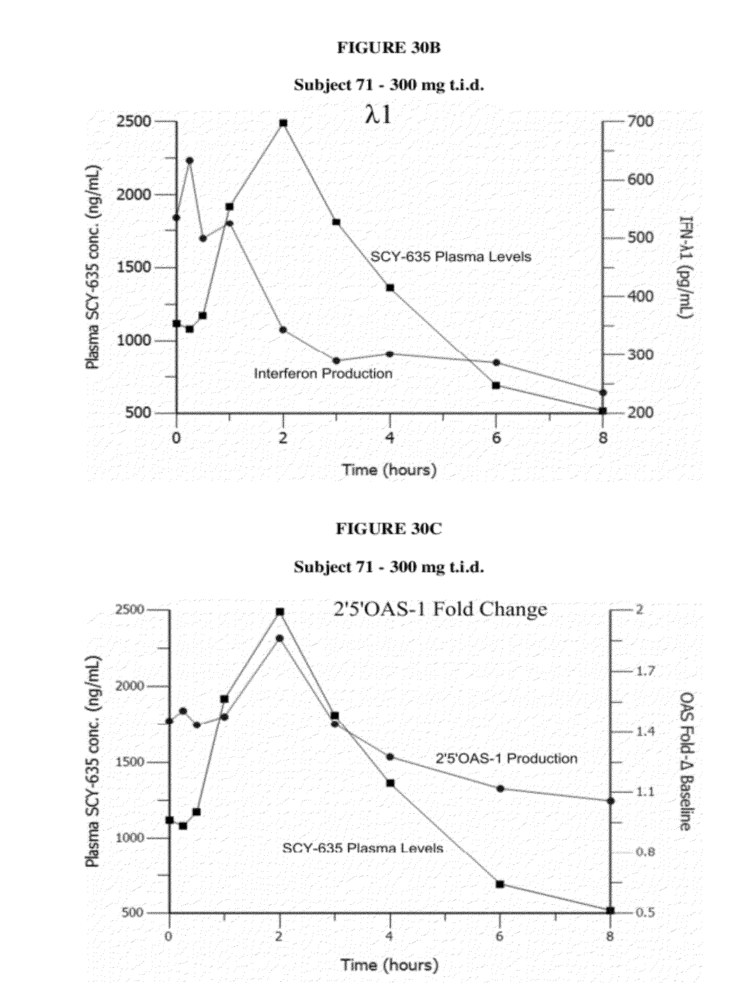

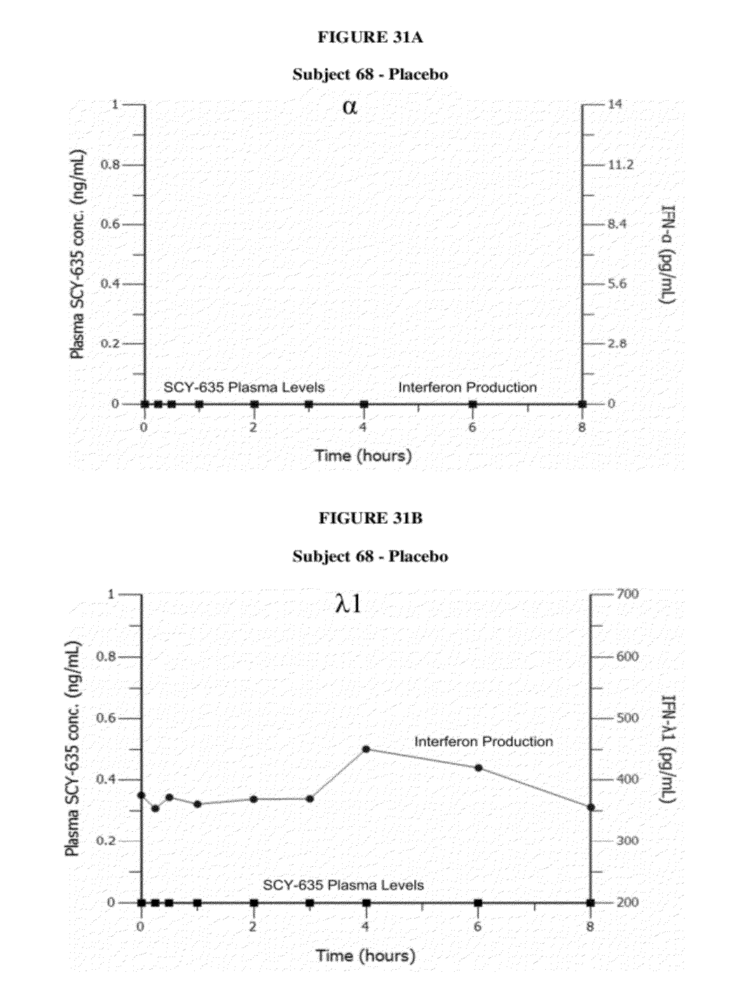

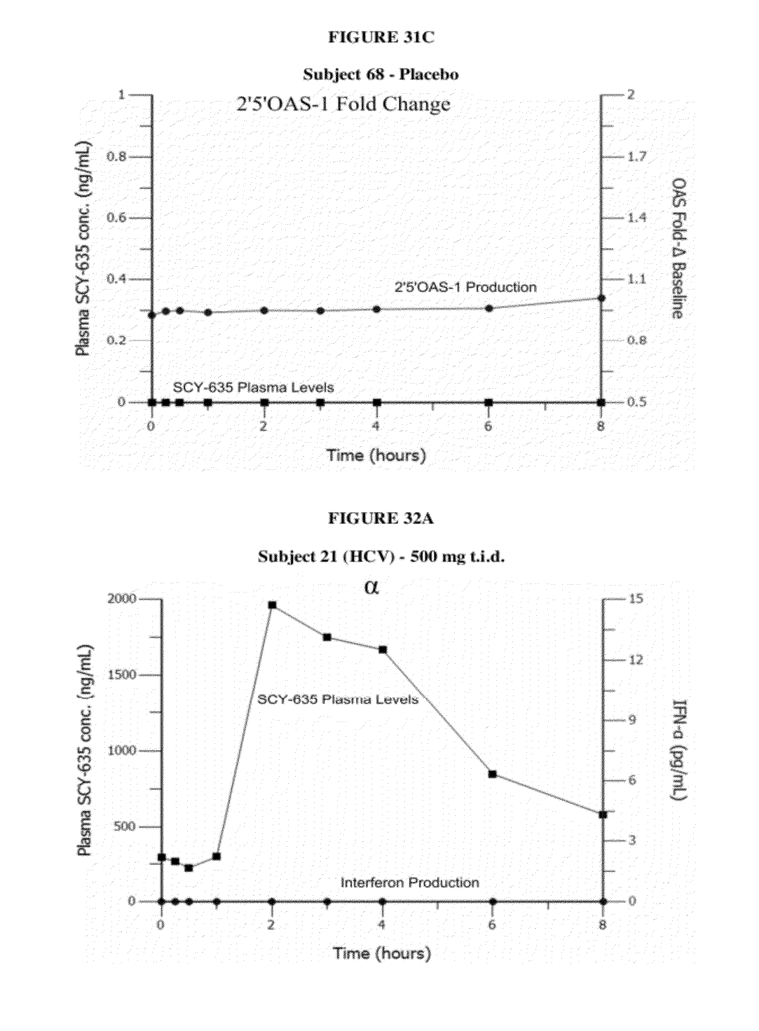

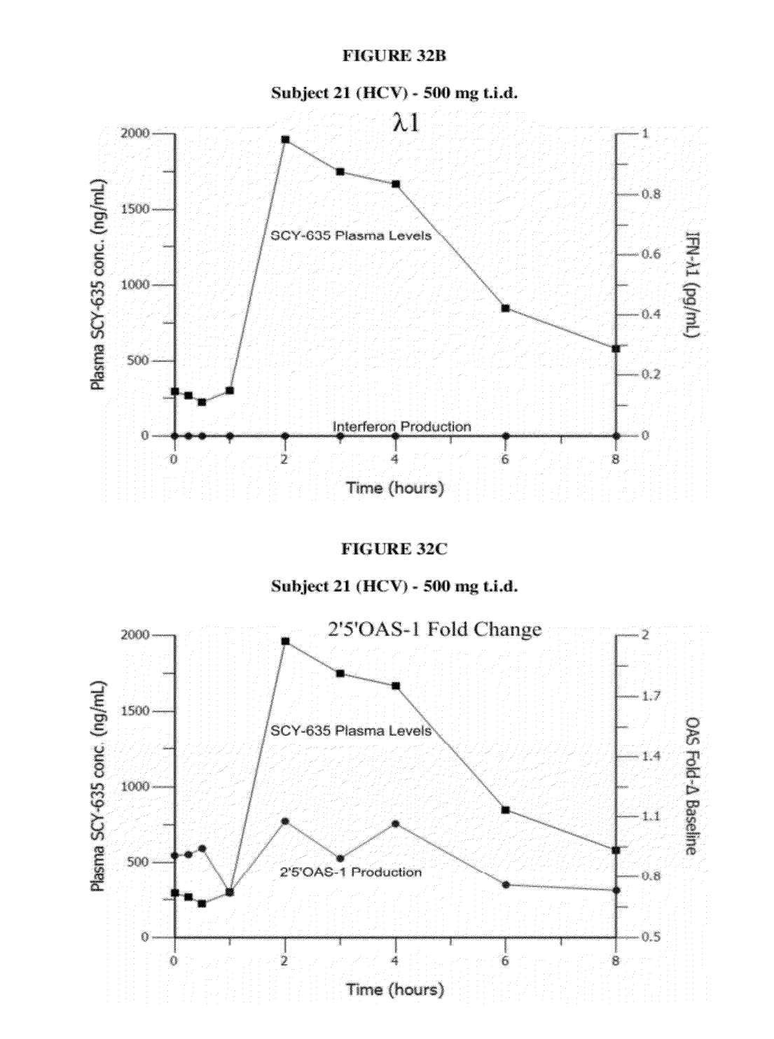

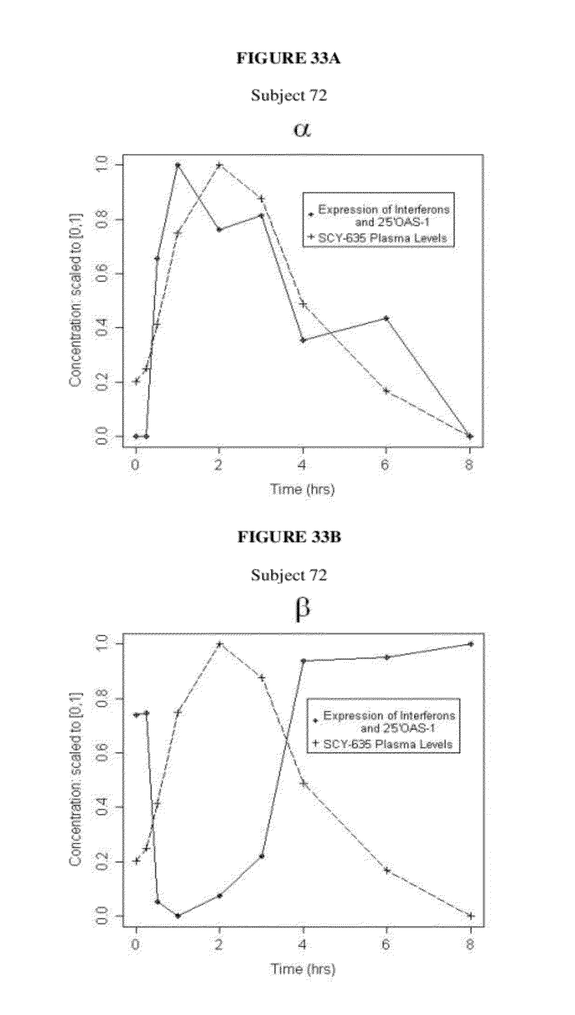

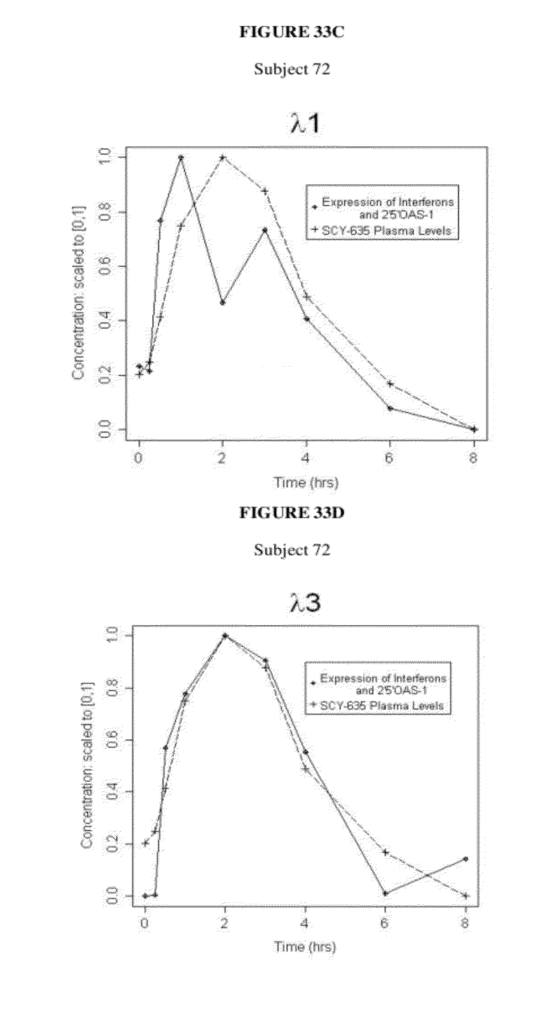

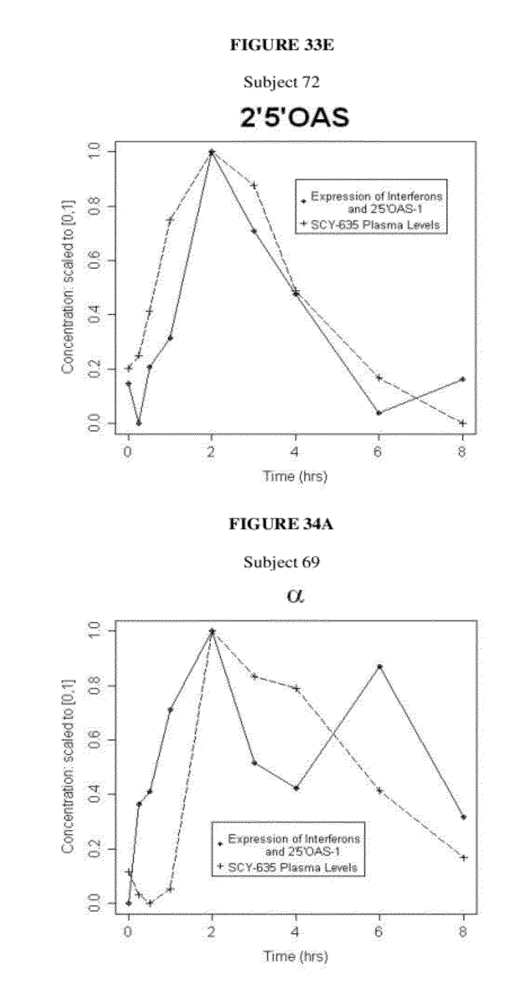

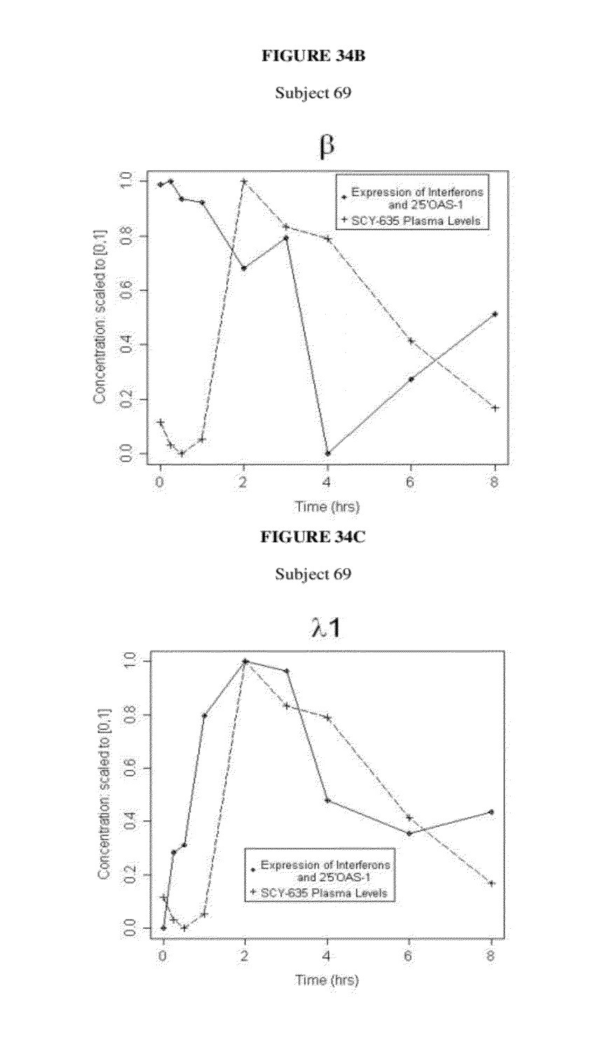

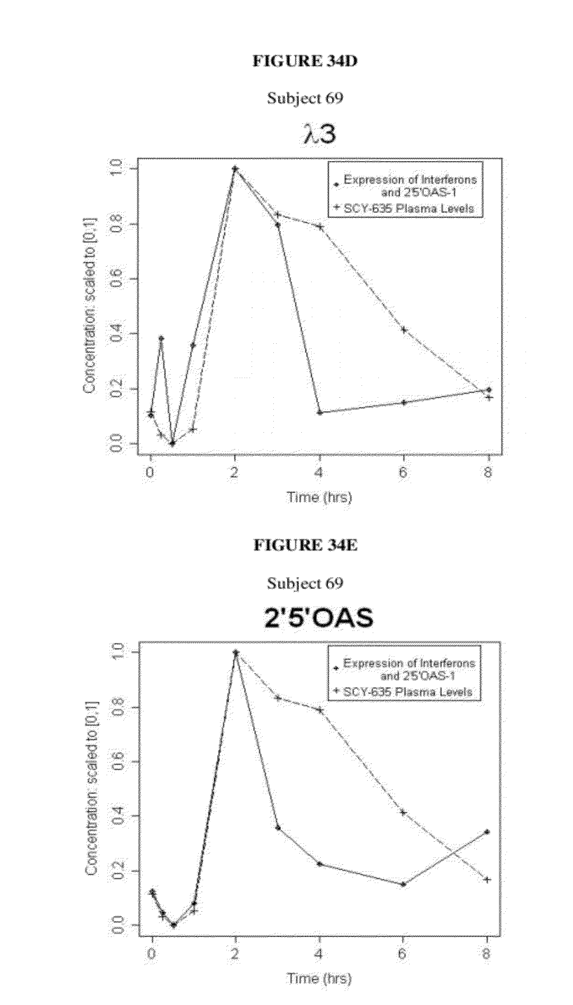

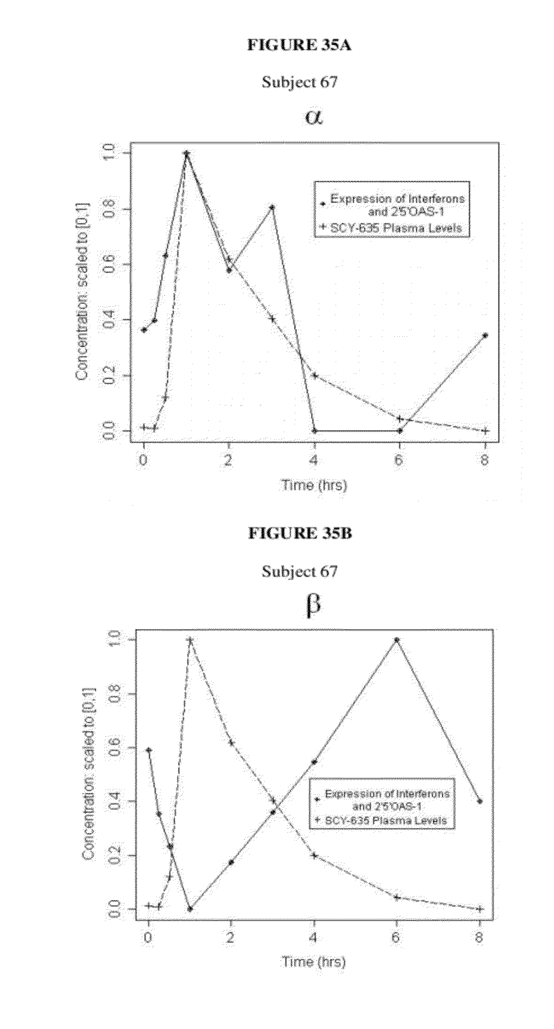

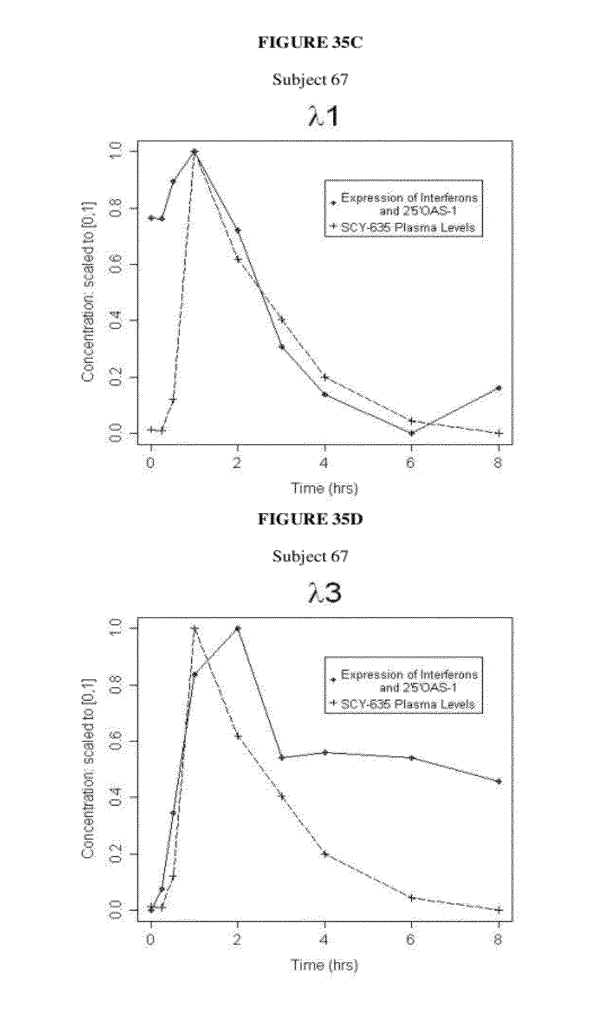

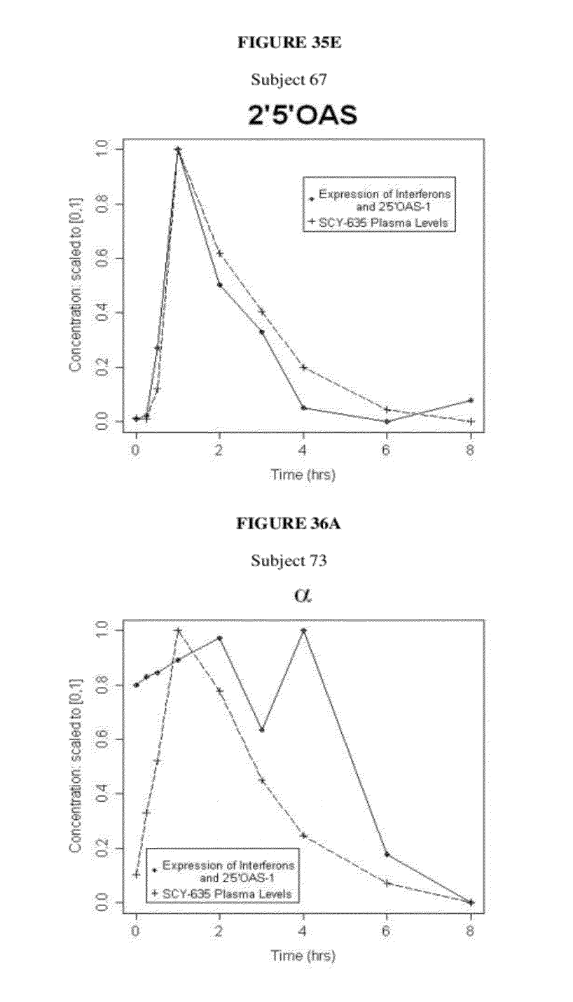

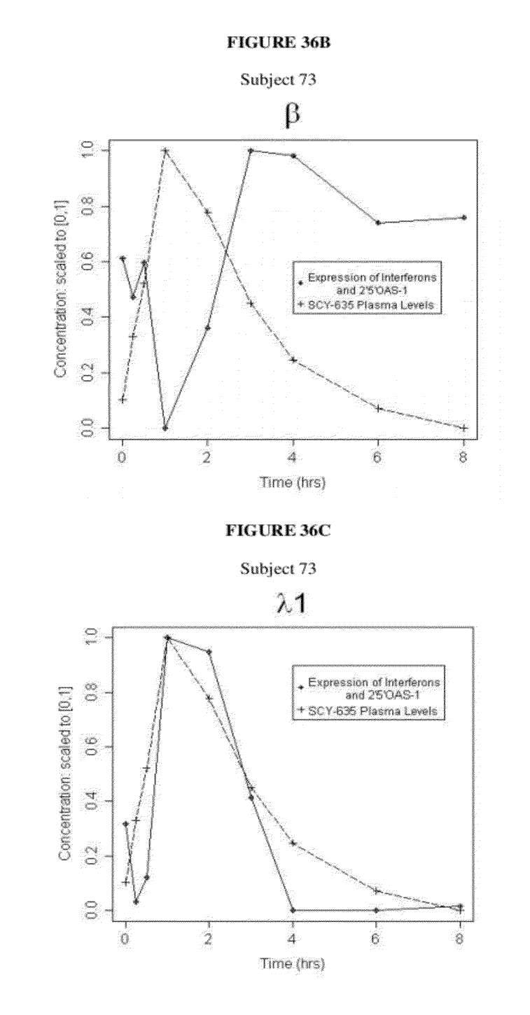

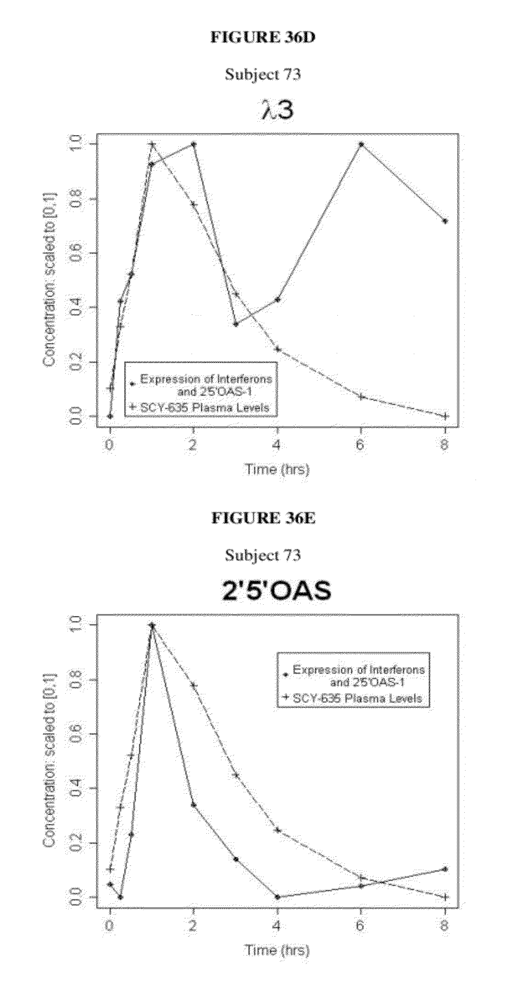

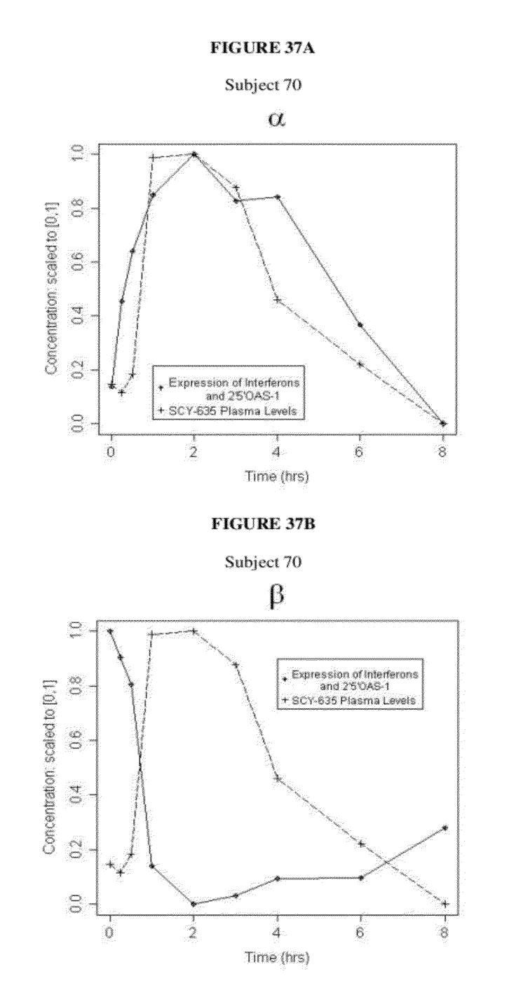

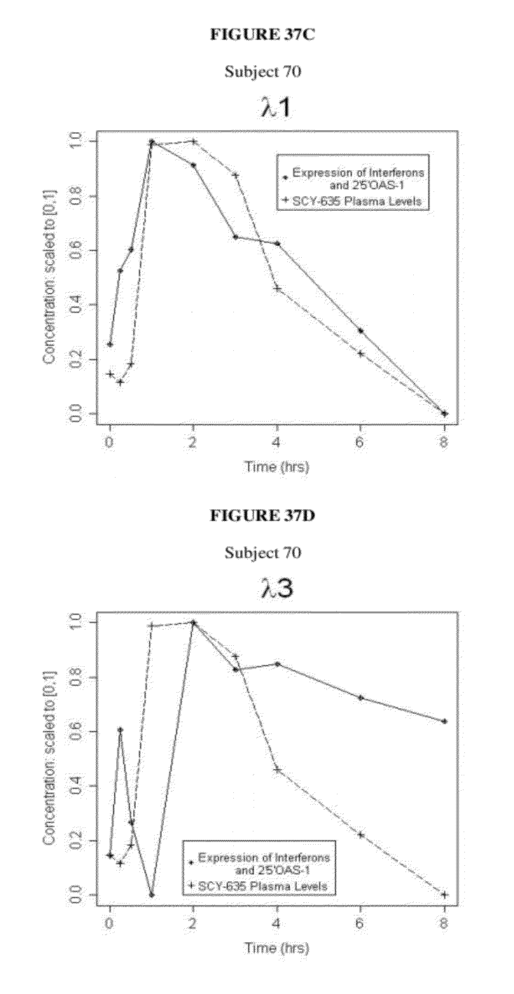

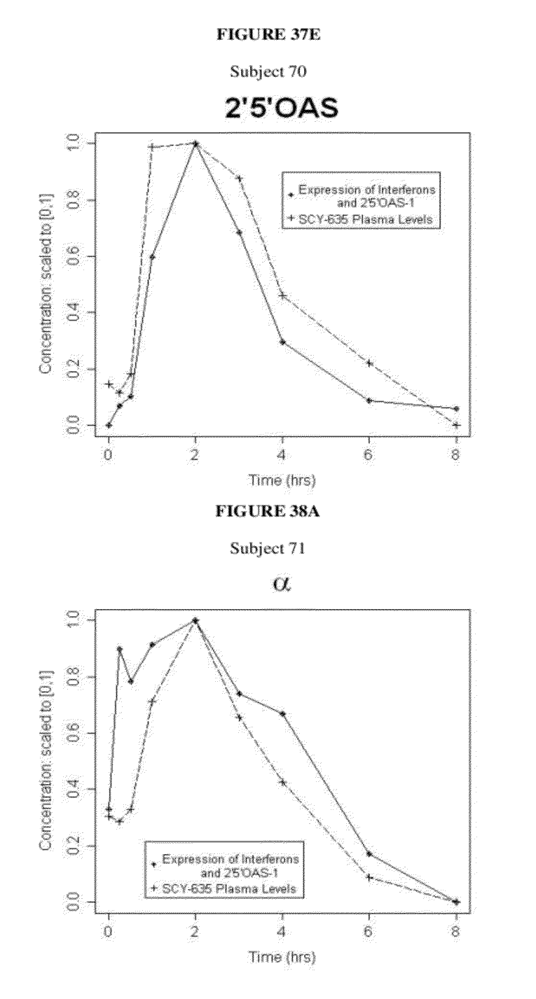

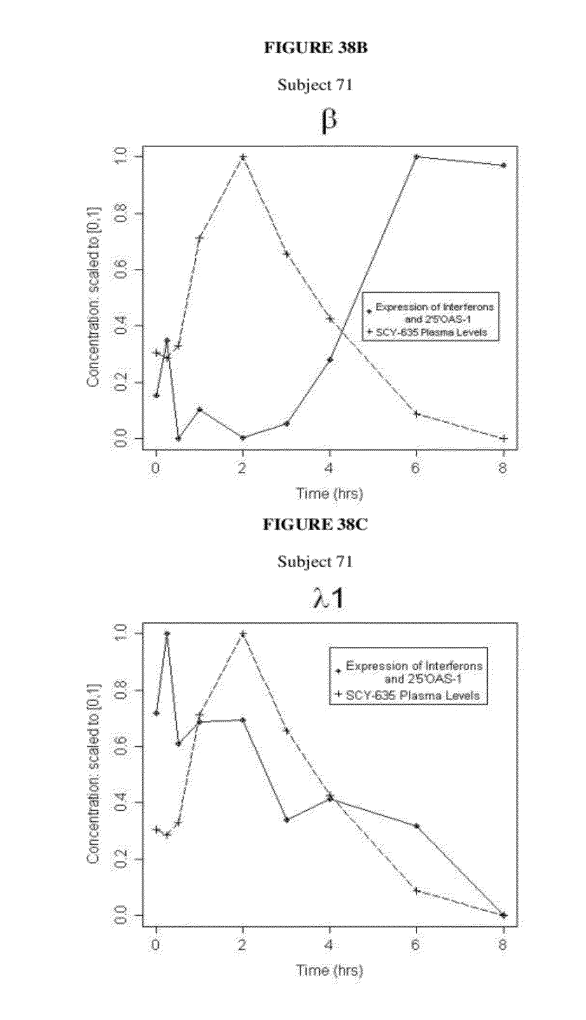

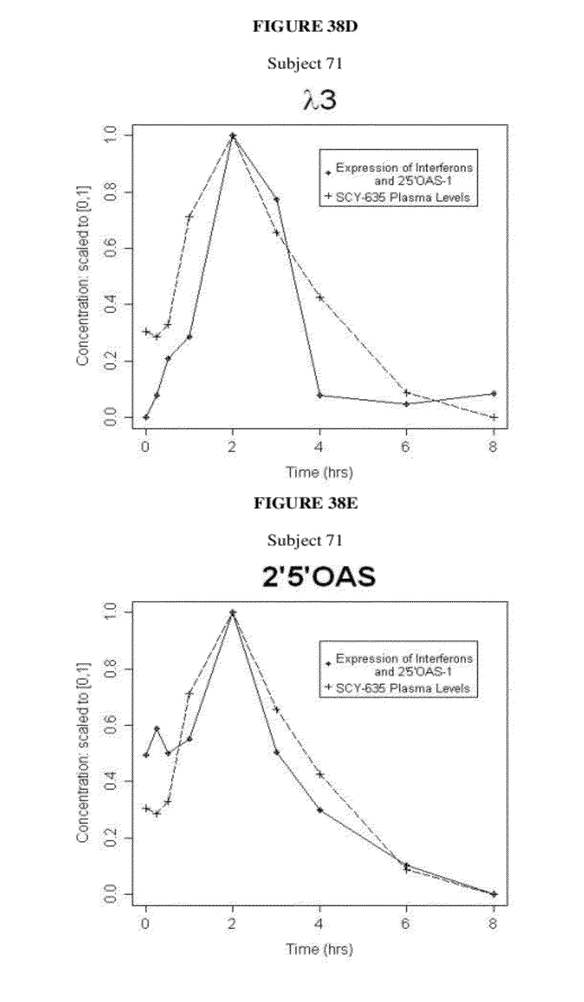

The group mean dose response for Cohorts 4, 5 and 6 is shown in FIG. 26. The individual viral load response for the subjects in Cohort 6 is shown in FIG. 27. FIGS. 28A-32C illustrate that interferon and 2′5′OAS-1 production is dependent upon the dose of SCY-635 and whether there is an HCV infection. FIGS. 33A through 38E illustrate the correlation between SCY-635 plasma levels and the expression of Type 1 and Type 3 interferons and 2′5′OAS-1 in Cohort 6 individuals.

Subject number, IL28B genotype, and maximum antiviral response for 6 subjects who received 900 milligrams SCY-635/day were 72/TT/−0.84; 69/CT/−1.42; 67/CT/−1.47; 73/CC/−2.34; 70/CT/2.44; 71/CC/−5.47. All subjects who received active treatment exhibited SCY-635-dependent increases in plasma protein concentrations of interferons α, λ1, and λ3 with concordant increases in the plasma protein concentration of 2′5; OAS1 and neopterin. Interindividual variability in antiviral responses exhibited an apparent correlation with interferon expression, immune activation, and IL28B genotype. CC and CT patients exhibited greater increases from baseline for endogenous interferons, 2′5′OAS-1, and neopterin followed by TT patients. Placebo subjects showed no consistent changes in interferon expression, no immune activation, and no significant change in HCV-specific plasma RNA.

SCY-635 exerts clinical antiviral activity by up regulating the expression of multiple endogenous interferons. The apparent correlation between IL28B genotypes and the magnitude of antiviral response to treatment with SCY-635 monotherapy suggests that the stimulation of pharmacologically relevant concentrations of endogenous interferons represents the primary mechanism through which SCY-635 exerts clinical anti-HCV activity.

TABLE 12
Correlation Coefficients for Subjects in Cohort 6: SCY-635
Concentrations vs. IFN a, b, l1, l3, and 2′5′OAS-1

| Subject | IFN a | IFN b | IFN l1 | IFN l3 | 2′5′OAS-1 |
|---|---|---|---|---|---|
| 72 | 0.8313 | −0.7867 | 0.7332 | 0.9370 | 0.8728 |
| 69 | 0.4997 | −0.5176 | 0.6591 | 0.6677 | 0.6421 |
| 67 | 0.7142 | −0.6730 | 0.4361 | 0.7717 | 0.9342 |
| 73 | 0.6240 | −0.7040 | 0.8549 | 0.3942 | 0.8405 |
| 70 | 0.8454 | −0.6492 | 0.8532 | 0.1539 | 0.9495 |
| 71 | 0.8005 | −0.7684 | 0.3981 | 0.8391 | 0.8580 |

The conclusions of these findings are highlighted below.
The kinetics, magnitude, and apparent correlation of host IL28B genotype to response indicates that treatment-associated secretion of multiple species of endogenous interferons drives the progressive decline in HCV-specific viremia following monotherapy with SCY-635.
The onset of antiviral activity observed at 900 mg/day coincides with the secretion of multiple Type I and Type III interferons which in turn leads to the increased expression of antiviral ISGs.
These data imply that cyclophilins regulate the expression of Type I and Type III interferons. SCY-635 exerts antiviral activity through a “de-repressor” mechanism.

All publications, patents and patent applications cited in this specification are herein incorporated by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A pharmaceutical composition for treating a patient having a disease susceptible to treatment with the cyclophilin-binding compound and a positive test for at least one cyclophilin-binding compound marker comprising a cyclophilin-binding compound, wherein the cyclophilin-binding compound marker is a polymorphism residing in a region within about 5 kilobases (kb) of the IL28B gene, encoding interferon-lambda-3.

2. A pharmaceutical composition for treating a patient having a disease susceptible to treatment with the cyclophilin-binding compound and a positive test for at least one cyclophilin-binding compound marker comprising a cyclophilin-binding compound, wherein the cyclophilin-binding compound marker is selected from among:

| | | | | Homozygous |
|---|---|---|---|---|
| | | Better | Heterozygous | cyclophilin- |
| | | Response | cyclophilin-binding | binding |
| PS | SNP | Allele | compound marker | compound marker |
| rs12979860 | C/T | C | C/T genotype | C/C genotype |
| rs28416813 | G/C | G | G/C genotype | G/G genotype |
| rs8103142 | A/G | A | A/G genotype | A/A genotype |
| rs12980275 | A/G | A | A/G genotype | A/A genotype |
| rs8099917 | A/C | A | A/C genotype | A/A genotype |
| rs12972991 | T/G | T | T/G genotype | T/T genotype |
| rs8109886 | A/C | C | C/A genotype | C/C genotype |
| rs4803223 | T/C | T | T/C genotype | T/T genotype |
| rs12980602 | A/G | A | A/G genotype | A/A genotype |

where the context of the SNP's is as follows

| | | Sequence |
|---|---|---|
| | | ID |
| PS | Short Context Sequence | No. |
| rs12979860 | CTGAACCAGGGAGCTCCCCGAAGGCG | 1 |
| | YGAACCAGGGTTGAATTGCACTCCGC | |
| rs28416813 | CAGAGAGAAAGGGAGCTGAGGGAATG | 2 |
| | SAGAGGCTGCCCACTGAGGGCAGGGG | |
| rs8103142 | TCCTGGGGAAGAGGCGGGAGCGGCAC | 3 |
| | YTGCAGTCCTTCAGCAGAAGCGACTC | |
| rs12980275 | CTGAGAGAAGTCAAATTCCTAGAAAC | 4 |
| | RGACGTGTCTAAATATTTGCCGGGGT | |
| rs8099917 | CTTTTGTTTTCCTTTCTGTGAGCAAT | 5 |
| | KTCACCCAAATTGGAACCATGCTGTA | |
| rs12972991 | AGAACAAATGCTGTATGATTCCCCCT | 6 |
| | MCATGAGGTGCTGAGAGAAGTCAAAT | |
| rs8109886 | TATTCATTTTTCCAACAAGCATCCTG | 7 |
| | MCCCAGGTCGCTCTGTCTGTCTCAAT | |
| rs4803223 | CCTAAATATGATTTCCTAAATCATAC | 8 |
| | RGACATATTTCCTTGGGAGCTATACA | |
| rs12980602 | TCATATAACAATATGAAAGCCAGAGA | 9 |
| | YAGCTCGTCTGAGACACAGATGAACA | |

3. The pharmaceutical composition according to claim 2, in which the cyclophilin-binding compound marker is a C/T polymorphism, identified as rs12979860 in the NCBI SNP Database.

4. The pharmaceutical composition according to claim 2, in which the cyclophilin-binding compound is cyclosporine A; a derivative of cyclosporine A; sanglifehrin A or a derivative of sanglifehrin A.

5. The pharmaceutical composition according to claim 4, in which the cyclophilin-binding compound is selected from the group consisting of cyclosporine A, alisporivir ([8-(N-methyl-D-alanine), 9-(N-ethyl-L-valine)]cyclosporin), (melle-4)cyclosporin (known as NIM-811) and 3-[(R)-2-(N,N-dimethylamino)ethylthio-Sar]-4-(gamma-hydroxymethylleucine)cyclosporin (SCY-635).

6. A method comprising administering to a human subject infected with hepatitis C virus an effective amount of a cyclophilin-binding compound, wherein an effective dosing regimen is selected according to the presence in the subject of a polymorphism residing in a region within about 5 kilobases (kb) of the IL28B gene, encoding interferon-lambda-3.

7. A method comprising administering to a human subject infected with hepatitis C virus an effective amount of a cyclophilin-binding compound, wherein an effective dosing regimen is selected according to the presence in the subject of at least one polymorphism selected from among:

| | | | | Homozygous |
|---|---|---|---|---|
| | | Better | Heterozygous | cyclophilin- |
| | | Response | cyclophilin-binding | binding |
| PS | SNP | Allele | compound marker | compound marker |
| rs12979860 | C/T | C | C/T genotype | C/C genotype |
| rs28416813 | G/C | G | G/C genotype | G/G genotype |
| rs8103142 | A/G | A | A/G genotype | A/A genotype |
| rs12980275 | A/G | A | A/G genotype | A/A genotype |
| rs8099917 | A/C | A | A/C genotype | A/A genotype |
| rs12972991 | T/G | T | T/G genotype | T/T genotype |
| rs8109886 | A/C | C | C/A genotype | C/C genotype |
| rs4803223 | T/C | T | T/C genotype | T/T genotype |
| rs12980602 | A/G | A | A/G genotype | A/A genotype |

where the context of the SNP's is as follows

| | | Sequence |
|---|---|---|
| | | ID |
| PS | Short Context Sequence | No. |
| rs12979860 | CTGAACCAGGGAGCTCCCCGAAGGCG | 1 |
| | YGAACCAGGGTTGAATTGCACTCCGC | |
| rs28416813 | CAGAGAGAAAGGGAGCTGAGGGAATG | 2 |
| | SAGAGGCTGCCCACTGAGGGCAGGGG | |
| rs8103142 | TCCTGGGGAAGAGGCGGGAGCGGCAC | 3 |
| | YTGCAGTCCTTCAGCAGAAGCGACTC | |
| rs12980275 | CTGAGAGAAGTCAAATTCCTAGAAAC | 4 |
| | RGACGTGTCTAAATATTTGCCGGGGT | |
| rs8099917 | CTTTTGTTTTCCTTTCTGTGAGCAAT | 5 |
| | KTCACCCAAATTGGAACCATGCTGTA | |
| rs12972991 | AGAACAAATGCTGTATGATTCCCCCT | 6 |
| | MCATGAGGTGCTGAGAGAAGTCAAAT | |
| rs8109886 | TATTCATTTTTCCAACAAGCATCCTG | 7 |
| | MCCCAGGTCGCTCTGTCTGTCTCAAT | |
| rs4803223 | CCTAAATATGATTTCCTAAATCATAC | 8 |
| | RGACATATTTCCTTGGGAGCTATACA | |
| rs12980602 | TCATATAACAATATGAAAGCCAGAGA | 9 |
| | YAGCTCGTCTGAGACACAGATGAACA | |

8. The method according to claim 7, in which the polymorphism is a C/T polymorphism, identified in the SNP rs12979860 in the NCBI SNP Database allele of the IL28b gene.

9. The method according to claim 7, in which the subject is infected with genotype 1 hepatitis C virus.

10. An assay method for evaluating the likelihood that a patient will respond to treatment by a cyclophilin-binding compound, said method comprising: (a) determining in a sample taken from patient the IL28B gene polymorphism residing in a region within about 5 kilobases (kb) of the IL28B gene, encoding interferon-lambda-3; (b) generating an efficacy index based upon the polymorphism of the gene; and (c) evaluating the likelihood that said subject will respond to the cyclophilin-binding compound based upon said efficacy index.

11. An assay method for evaluating the likelihood that a patient will respond to treatment by a cyclophilin-binding compound, said method comprising:
(a) obtaining a sample taken from patient,
(b) determining a polymorphism;
(c) generating an efficacy index based upon the polymorphism of the gene; and
(d) evaluating the likelihood that said subject will respond to the cyclophilin-binding compound based upon said efficacy index
wherein the efficacy index is determined according to the presence in the subject of at least one polymorphism selected from among:

| | | | | Homozygous |
|---|---|---|---|---|
| | | Better | Heterozygous | cyclophilin- |
| | | Response | cyclophilin-binding | binding |
| PS | SNP | Allele | compound marker | compound marker |
| rs12979860 | C/T | C | C/T genotype | C/C genotype |
| rs28416813 | G/C | G | G/C genotype | G/G genotype |
| rs8103142 | A/G | A | A/G genotype | A/A genotype |
| rs12980275 | A/G | A | A/G genotype | A/A genotype |
| rs8099917 | A/C | A | A/C genotype | A/A genotype |
| rs12972991 | T/G | T | T/G genotype | T/T genotype |
| rs8109886 | A/C | C | C/A genotype | C/C genotype |
| rs4803223 | T/C | T | T/C genotype | T/T genotype |
| rs12980602 | A/G | A | A/G genotype | A/A genotype |

where the context of the SNP's is as follows

| | | Sequence |
|---|---|---|
| | | ID |
| PS | Short Context Sequence | No. |
| rs12979860 | CTGAACCAGGGAGCTCCCCGAAGGCG | 1 |
| | YGAACCAGGGTTGAATTGCACTCCGC | |
| rs28416813 | CAGAGAGAAAGGGAGCTGAGGGAATG | 2 |
| | SAGAGGCTGCCCACTGAGGGCAGGGG | |
| rs8103142 | TCCTGGGGAAGAGGCGGGAGCGGCAC | 3 |
| | YTGCAGTCCTTCAGCAGAAGCGACTC | |
| rs12980275 | CTGAGAGAAGTCAAATTCCTAGAAAC | 4 |
| | RGACGTGTCTAAATATTTGCCGGGGT | |
| rs8099917 | CTTTTGTTTTCCTTTCTGTGAGCAAT | 5 |
| | KTCACCCAAATTGGAACCATGCTGTA | |
| rs12972991 | AGAACAAATGCTGTATGATTCCCCCT | 6 |
| | MCATGAGGTGCTGAGAGAAGTCAAAT | |
| rs8109886 | TATTCATTTTTCCAACAAGCATCCTG | 7 |
| | MCCCAGGTCGCTCTGTCTGTCTCAAT | |
| rs4803223 | CCTAAATATGATTTCCTAAATCATAC | 8 |
| | RGACATATTTCCTTGGGAGCTATACA | |
| rs12980602 | TCATATAACAATATGAAAGCCAGAGA | 9 |
| | YAGCTCGTCTGAGACACAGATGAACA | |

12. The assay method according to claim 11, in which the patient is infected with genotype 1 hepatitis C virus.

13. A method of treating a patient infected with a viral disease, the method comprising determining an efficacy index according to claim 11 and administering an effective dose of a cyclophilin-binding compound selected according to the efficacy index.

14. The method according to claim 13, wherein said viral disease is HCV.

15. A method of treating a patient infected with a viral disease, the method comprising determining an efficacy index according to claim 11 and administering an effective dose of a cyclophilin binding compound selected according to the efficacy index.

16. The method according to claim 15, wherein said viral disease is HCV.

17. The method according to claim 16, wherein the patient is infected with genotype 1 HCV.

18. The method according to claim 13, wherein said efficacy index is compared to an index cutoff value.

19. The method according to claim 13, wherein an efficacy index greater than said index cutoff value indicates that said subject does not have a high likelihood of responding to the cyclophilin-binding compound.

20. The method according to claim 13, wherein said sample is selected from the group consisting of whole blood, serum, plasma, and buccal cells.

21. A method of expressing endogenous interferon in a cell infected with a virus, said method comprising treating said cell with an effective amount of at least one cyclophilin inhibitor.

22. The method according to claim 21 in which the at least one interferon is selected from the group consisting of interferon alpha, interferon lambda-1 and interferon lambda-3.

23. The method according to claim 21, wherein interferon beta production is down-regulated.

24. The method according to claim 21, where the cell is infected with a hepatitis virus.

25. The method according to claim 23, where the hepatitis virus is HCV.

26. The method according to claim 21, where the cyclophilin inhibitor is cyclosporine A or a derivative thereof.

27. The method according to claim 26, where the cyclophilin inhibitor is a non-immunosuppressive cyclophilin inhibitor.

28. The method according to claim 27, where the non-immunosuppressive cyclophilin inhibitor is selected from the group consisting of alisporivir, NIM-811 and SCY-635.

29. The method according to claim 21, where the cyclophilin inhibitor is sanglifehrin A or a derivative thereof.


SEQUENCE LISTING (9 sequences)

SEQ ID NO: 1 - DNA, 52 bp, Homo sapiens
  misc_feature (27)..(27): Y indicates C or T
ctgaaccagggagctccccgaaggcgygaaccagggttgaattgcactccgc

SEQ ID NO: 2 - DNA, 52 bp, Homo sapiens
  misc_feature (27)..(27): S indicates G or C
cagagagaaagggagctgagggaatgsagaggctgcccactgagggcagggg

SEQ ID NO: 3 - DNA, 52 bp, Homo sapiens
  misc_feature (27)..(27): Y indicates C or T
tcctggggaagaggcgggagcggcacytgcagtccttcagcagaagcgactc

SEQ ID NO: 4 - DNA, 52 bp, Homo sapiens
  misc_feature (27)..(27): R indicates G or A
ctgagagaagtcaaattcctagaaacrgacgtgtctaaatatttgccggggt

SEQ ID NO: 5 - DNA, 52 bp, Homo sapiens
  misc_feature (27)..(27): K = G or T
cttttgttttcctttctgtgagcaatktcacccaaattggaaccatgctgta

SEQ ID NO: 6 - DNA, 52 bp, Homo sapiens
  misc_feature (27)..(27): M = A or C
agaacaaatgctgtatgattccccctmcatgaggtgctgagagaagtcaaat

SEQ ID NO: 7 - DNA, 52 bp, Homo sapiens
  misc_feature (27)..(27): M = A or C
tattcatttttccaacaagcatcctgmcccaggtcgctctgtctgtctcaat

SEQ ID NO: 8 - DNA, 52 bp, Homo sapiens
  misc_feature (27)..(27): R indicates G or A
cctaaatatgatttcctaaatcatacrgacatatttccttgggagctataca

SEQ ID NO: 9 - DNA, 52 bp, Homo sapiens
  misc_feature (27)..(27): Y indicates C or T
tcatataacaatatgaaagccagagayagctcgtctgagacacagatgaaca